(19) United States
(12) Patent Application Publication
(10) Pub. No.: US 2024/0408114 A1
(43) Pub. Date: Dec. 12, 2024

(54) METHODS FOR PREVENTING AND TREATING ACUTE KIDNEY INJURY

(71) Applicant: The Johns Hopkins University, Baltimore, MD (US)

(72) Inventors: Hamid RABB, Ellicott City, MD (US); Barbara SLUSHER, Kingsville, MD (US); Jonathan D. POWELL, Baltimore, MD (US); Kyungho LEE, Woodstock, MD (US); Elizabeth THOMPSON, Phoenix, MD (US); Sanjeev NOEL, Baltimore, MD (US); Sepideh GHARAIE, Nottingham, MD (US)

(21) Appl. No.: 18/689,200

(22) PCT Filed: Nov. 4, 2022
(86) PCT No.: PCT/US2022/079313

§ 371 Date: Mar. 5, 2024

Related U.S. Application Data
(60) Provisional application No. 63/275,692, filed on Nov. 4, 2021.

(51) Int. Cl. A61K 31/655; A61K 31/664; A61K 31/675; A61K 31/7034; A61K 31/7076; A61P 13/12

(52) U.S. Cl. CPC A61K 31/655; A61K 31/664; A61K 31/675; A61K 31/7034; A61K 31/7076; A61P 13/12

(57) ABSTRACT

Provided are glutamine antagonists and prodrugs of glutamine analogs having formula (I):

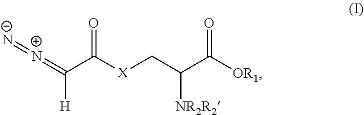

and pharmaceutically acceptable salts thereof, wherein R₁, R₂, R₂′, and X are as defined as set forth in the specification, for use in preventing and/or treating acute kidney injury.

### FEDERALLY SPONSORED RESEARCH OR DEVELOPMENT

This invention was made with government support under grants DK123342 and DK104662 awarded by the National Institutes of Health. The government has certain rights in the invention.

### BACKGROUND

Acute kidney injury (AKI) is a common problem in native and transplanted kidneys. No specific treatment for AKI is currently available. T cells have been found previously to mediate AKI. Mechanisms by which T cells mediate AKI and repair have been studied over the course of the past 20 years. More recently, it has been found that T cells undergo metabolic reprogramming during AKI in mice and humans.

### SUMMARY

In some aspects, the presently disclosed subject matter provides method for preventing or treating a subject afflicted with, suspected of having, or susceptible to having an acute kidney injury, the method comprising administering to the subject at least one glutamine antagonist, or a prodrug or analog thereof, in an amount effective to prevent or treat the acute kidney injury.

In some aspects, the prodrug of the at least one glutamine antagonist comprises a compound of formula (I):

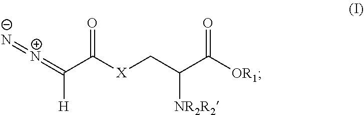

or a pharmaceutically acceptable salt thereof, wherein:
X is selected from the group consisting of a bond, —O—, and —(CH₂)ₙ—, wherein n is an integer selected from the group consisting of 1, 2, 3, 4, 5, 6, 7, and 8;
R₁ is selected from the group consisting of C₁₋₆ alkyl and substituted C₁₋₆ alkyl;
R₂ is —C(═O)—O—(CR₃R₄)ₘ—O—C(═O)—R₁₀;
R₂′ is selected from the group consisting of H, C₁-C₆ alkyl, and substituted C₁-C₆ alkyl;
each R₃ and R₄ is independently H, C₁-C₆ alkyl, substituted C₁-C₆ alkyl, aryl, substituted aryl, —(CR₃R₄)ₘ—NR₅R₆, or

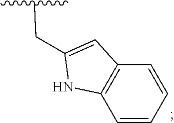 [SMILES: *Cc1cc2ccccc2[nH]1]

m is an integer selected from the group consisting of 1, 2, 3, 4, 5, 6, 7, and 8;
R₅ and R₆ is independently H or alkyl; and
R₁₀ is selected from the group consisting of alkyl, substituted alkyl, cycloalkyl, substituted cycloalkyl, monosaccharide, acylated monosaccharide, aryl, substituted aryl, heteroaryl, and substituted heteroaryl,
in an amount effective to treat the acute kidney injury.

In particular aspects, the prodrug of the at least one glutamine antagonist is

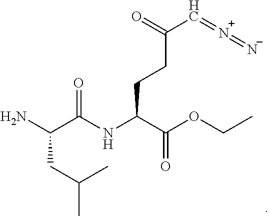 [SMILES: CCOC(=O)[C@H](CCC(=O)C=[N+]=[N-])NC(=O)[C@@H](N)CC(C)C]

In certain aspects, the administering of the at least one glutamine antagonist, or a prodrug or analog thereof, modulates a glutamine pathway associated with acute kidney injury. In particular aspects, the modulation of the glutamine pathway alters T cell metabolism. In more certain aspects, the modulation of the glutamine pathway blocks glutaminolysis.

In certain aspects, blockade of glutaminolysis reduces T cell activation and proliferation in post-ischemia reperfusion injury (IRI) kidneys. In certain aspects, blockade of glutaminolysis reduces CD69 expression in post-ischemia reperfusion injury (IRI) kidney CD8⁺ cells.

In certain aspects, the administering of the at least one glutamine antagonist, or a prodrug or analog thereof, causes a decrease in CD44, CD4⁺, and CD8⁺ T cells in post-IRI kidneys.

In certain aspects, the administering of the at least one glutamine antagonist, or a prodrug or analog thereof, causes a reduced expression of Ki67 cells in post-IRI kidneys.

In certain aspects, the administering of the at least one glutamine antagonist, or a prodrug or analog thereof, enhances expression of hexokinase II and pS6 on one or more kidney T cells.

In some aspects, the acute kidney injury is caused by ischemia or an ischemic event, direct injury to the kidney, blockage of a urinary tract, or combinations thereof. In certain aspects, the direct injury to the kidney is caused by exposure to one or nephrotoxins or from a disease or condition. In particular aspects, the one or more nephrotoxins include a chemotherapeutic agent, an antibiotic, an NSAID, a gold preparation, a thiazide, a sulfonamide, an aminoglycosides, an ACE inhibitor, an angiotensin II antagonist, an antiviral, vancomycin, ranitidine, amphotericin B, and combinations thereof. In particular aspects, the disease or condition includes sepsis, a cancer, vasculitis, interstitial nephritis, scleroderma, tubular necrosis, glomerulonephritis, thrombotic microangiopathy, and combinations thereof.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1 is a cartoon illustrating that T cells undergo metabolic reprogramming during ischemic acute kidney injury (AKI);

FIG. 2 is a cartoon illustrating that modulation of the glutamine pathway alters T cell activation and function, which affects renal outcomes;

FIG. 3 shows the metabolic pathways and markers studied in the presently disclosed subject matter;

FIG. 4 demonstrates that a metabolically distinct subset of T cells with VDAC1^{low} and phospho-S6^{low} increased in kidneys after ischemic AKI;

FIG. 5 demonstrates that H3K27Me3⁺ T cells increase in human and mouse kidneys during ischemia;

FIG. 6 demonstrates that splenic T cells are metabolically activated following ischemic AKI;

FIG. 7 shows that glutamine blockade by JHU083 treatment attenuated renal injury and altered T cell metabolism;

FIG. 8 shows that glutamine blockade by JHU083 reduced kidney T cell activation and proliferation after ischemic AKI;

FIG. 9A and FIG. 9B show the metabolic pathways studied and metabolic signature of in vivo activated kidney T cells (FIG. 9A) We evaluated key enzymes for glycolysis, fatty acid oxidation, and mitochondrial oxidative phosphorylation, as well as histone methylation marker. Glucose transporter GLUT1 and hexokinase II were used to evaluate glycolysis machinery. CPT1a expressions was measured for fatty acid oxidation. mTOR signaling activity was measured with S6 ribosomal protein phosphorylation. Mitochondrial oxidative phosphorylation was measured with VDAC1 and Tomm20. H3K27me3 was measured as a readout for histone methylation. (FIG. 9B) Histograms comparing kidney T cells from control mice (gray) and LCMV infected mice (green) as a positive control (on day 7 after LCMV inoculation). CPT1a, carnitine palmitoyltransferase 1a; LCMV, lymphocytic choriomeningitis virus; pS6, phospho-S6 ribosomal protein; VDAC1, voltage-dependent anion channel 1;

FIG. 10A, FIG. 10B, FIG. 10C, FIG. 10D, and FIG. 10E show metabolically distinct subset of T cells with VDAC1- and pS6- increases in kidneys following ischemic AKI. (FIG. 10A) Schematic of the experimental design. (FIG. 10B) Concatenated flow cytometry data depicted as UMAP projection of T cells in control kidneys, ischemic kidneys and post-IRI kidneys. Post-IRI kidneys showed distinct segregated populations having low VDAC1 and pS6 expressions compared to the control kidneys and ischemic kidneys. (FIG. 10C) Representative flow plots showing VDAC1^{low} pS6^{low} T cells. Frequencies of VDAC1^{low} pS6^{low}T subsets among CD4⁺, CD8⁺, and DN T cells in mouse kidneys. They were significantly increased following reperfusion. (FIG. 10D) Histograms comparing VDAC1^{low} pS6^{low}T cells (blue) and remaining T cells (brown) from concatenated post-IRI 48 h data. (FIG. 10E) Changes in glycolysis enzymes on VDAC1^{low} pS6^{low} T cells according to different time points. GLUT1 and hexokinase II on those cells were increased significantly following TRI. *P<0.05, compared with the control group, †P<0.05, compared with the ischemia group, P<0.05, compared with the post-IRI 4 h group (n=10 mice in each group). Statistical analyses were performed using ANOVA followed by Tukey's post-hoc analysis. Data are from two independent experiments. CPT1a, carnitine palmitoyltransferase 1a; DN, double-negative; IRI, ischemia-reperfusion injury; pS6, phospho-S6 ribosomal protein; UMAP, Uniform Manifold Approximation and Projection; VDAC1, voltage-dependent anion channel 1;

FIG. 11A, FIG. 11B, and FIG. 11C demonstrate that splenic T cells have higher metabolic activity following ischemic AKI. (FIG. 11A) Unbiased UMAP analysis of concatenated flow cytometry data of splenic T cells from control mice (gray and black), mice during renal ischemia (red), and post-renal IRI 4 h (blue) and 48 h (purple) mice. Multiple enzymes associated with glycolysis, fatty acid oxidation, and oxidative phosphorylation drove the separation of splenic T cells, showing that metabolically activated T cell subsets were increased in the spleens following renal IRI (arrows). (FIG. 11B) Histograms comparing splenic T cells from control mice (gray) and post-IRI mice (48 h) (purple). (FIG. 11C) Normalized MFI of significantly changed metabolic markers on splenic CD4⁺, CD8⁺, and DN T cells. *P<0.05, compared with the control group; †P<0.05, compared with the renal ischemia group; P<0.05, compared with the post-renal IRI 4 h group (n=10 mice in each group). Statistical analyses were performed using ANOVA followed by Tukey's post hoc analysis. Data are from two independent experiments. AKI, acute kidney injury; CPT1a, carnitine palmitoyltransferase 1a; DN, double-negative; IRI, ischemia-reperfusion injury; UMAP, Uniform Manifold Approximation and Projection;

FIG. 12 demonstrates the effect of in vitro hypoxia on kidney T cell metabolism. FACS sorted kidney T cells were cultured under CD3/CD28 stimulation and exposed to hypoxia for 24 h followed by reoxygenation. T cells exposed to hypoxia showed higher levels of hexokinase II and GLUT1 expression. *P<0.05, **P<0.01, ***P<0.001, and ****P<0.0001. Statistical analyses were performed using T-test;

FIG. 13A, FIG. 13B, FIG. 13C, and FIG. 13D show that H3K27Me3⁺ T cells increase in human kidneys during ischemia. (FIG. 13A) Concatenated flow cytometry data depicted as UMAP projection of T cells from human non-ischemic kidneys (gray) and ischemic kidneys (red). H3K27Me3 expression drove separation of the non-ischemic and ischemic cluster. (FIG. 13B) Representative flow plots of human non-ischemic kidney and ischemic kidneys depicting expression of H3K27Me3⁺ (FIG. 13C) Frequencies of H3K27Me3⁺ subsets among CD4⁺, CD8⁺, and DN T cells in human kidneys. T cells in ischemic kidneys showed significantly higher frequencies of H3K27Me3⁺ cells compared to those of non-ischemic kidneys. (FIG. 13D) Frequencies of H3K27Me3⁺ subsets among CD4⁺, CD8⁺, and DN T cells in mouse kidneys, showing consistent findings with human data. *P<0.05, **P<0.01, ***P<0.001, and ****P<0.0001 (n=3 in each group for human subjects; n=10 in each group for mice). Statistical analyses were performed using T-test. Mouse data are from two independent experiments. DN, double-negative; UMAP, Uniform Manifold Approximation and Projection;

FIG. 14A, FIG. 14B, FIG. 14C, and FIG. 14D demonstrate the effect of glutamine blockade in ischemic AKI. (FIG. 14A) Schematic of the experimental design. (FIG. 14B) Glutaminase activity in post-ischemic kidneys was significantly reduced in the JHU083 treated mice compared to the vehicle treated mice (n=5-6 mice in each group). (FIG. 14C) Plasma creatinine concentrations following ischemic AKI. JHU083 treatment significantly attenuated functional renal injury. n=16 mice in each group. Two mice from the vehicle control group and one mouse from the JHU083 treated group died on day 2 following IRI. Data are from three independent experiments. (FIG. 14D) Histologic findings at 24 h after ischemic AKI. Necrotic tubules in renal cortex were significantly lower in the JHU083 treated group compared to the vehicle control group. n=10 mice in each group, data are from two independent experiments *P<0.05, **P<0.01, Statistical analyses were performed using T-test. AKI, acute kidney injury; GLS, glutaminase; IRI, ischemia-reperfusion injury;

FIG. 15A, FIG. 15B, FIG. 15C, and FIG. 15D demonstrate the effects of glutamine blockade on T cell activation and proliferation in post-ischemic kidneys. (FIG. 15A and FIG. 15B) JHU083 treatment resulted in significant decrease in CD44 expression and increase in CD62L expression in kidney CD4⁺ and CD8⁺ T cells, suggesting that JHU083 treated post-ischemic kidneys had less effector memory phenotype T cells. (FIG. 15C) Activation marker CD69 expression levels on CD8⁺ T cells were reduced in the JHU083 treated group. (FIG. 15D) Proliferation marker Ki67 expression in CD4⁺ and CD8⁺ T cells were downregulated in the JHU083 treated group. *P<0.05, **P<0.01, and ***P<0.001 (n=9 mice in each group). Statistical analyses were performed using T-test. DN, double-negative;

FIG. 16A, FIG. 16B, and FIG. 16C demonstrate the effect of glutamine blockade in cisplatin-induced AKI. (FIG. 16A) Schematic of the experimental design. (FIG. 16B) Plasma creatinine concentrations following cisplatin-induced AKI. JHU083 treatment significantly attenuated functional renal injury. n=9 mice in each group. Two mice died in the vehicle control group on day 2. (FIG. 16C) Histologic findings at 72 h after cisplatin-induced AKI. Necrotic tubules in outer medulla were significantly lower in the JHU083 treated group. n=7-9 mice in each group. *P<0.05, **P<0.01, ***P<0.001, and ****P<0.0001. Statistical analyses were performed using T-test. AKI, acute kidney injury;

FIG. 17A, FIG. 17B, FIG. 17C, and FIG. 17D demonstrate the effects of glutamine blockade on T cell activation and proliferation in cisplatin AKI. (FIG. 17A and FIG. 17B) JHU083 treatment resulted in significantly reduced CD44 expression and increased CD62L expression in kidney CD4⁺ and CD8⁺ T cells. (FIG. 17C) CD69 expression in CD4⁺, CD8⁺, and DN T cells were reduced. (FIG. 17C) Percentage of CD62L expression was increased in CD4⁺ and CD8⁺ T cells. (FIG. 17D) Ki67 expression in CD4⁺ and CD8⁺ T cells was reduced. *P<0.05, **P<0.01, and ***P<0.001 (n=7-9 mice in each group). Statistical analyses were performed using T-test. DN, double-negative;

FIG. 18A, FIG. 18B, and FIG. 18C demonstrate the effects of glutamine blockade on T cell activation and proliferation in vitro hypoxia. CD3/CD28 stimulated T cells were treated with JHU083 or vehicle and underwent 24 h hypoxia followed by reoxygenation. (FIG. 18A) JHU083 treatment reduced CD44, CD69, CD25, and Ki67 expression with a dose dependent manner. (FIG. 18B) Histograms comparing vehicle treated T cells and JHU083 treated T cells. (FIG. 18C) CD3/CD28 stimulated kidney T cells were exposed to hypoxia followed by reoxygenation. JHU083 treatment reduced kidney T cell proliferation significantly. *P<0.05, **P<0.01, and ***P<0.001. Statistical analyses were performed using one-way ANOVA followed by Tukey post-hoc analysis or T-test. DN, double-negative;

FIG. 19 shows the gating strategy for kidney T cells. Isolated KMNCs were analyzed with spectral flow cytometer as described in Methods. After single cell gating, lymphocytes were identified with SSC-A and CD45 high gating. αβ T cells were identified from live lymphocytes population with TCRβ⁺ gating. KMNCs, kidney mononuclear cells;

FIG. 20 demonstrates the effects of glutamine blockade on kidney T cell populations in post-ischemic kidneys. Post-ischemic kidneys from the JHU083 treated mice had a reduced number of total T cells. Proportions of CD4⁺, CD8⁺, and DN T cells among total T cells were comparable between groups. Percentage of Tregs among CD4⁺ T cells was comparable. *P<0.05, **P<0.01, and ***P<0.001. Statistical analyses were performed using T-test. DN, double-negative;

FIG. 21 demonstrates the effects of glutamine blockade on splenic T cell phenotypes in ischemic AKI. JHU083 treatment reduced CD44 expression and increased CD62L expression in splenic CD4⁺ and CD8 T⁺ cells from post-IRI mice. *P<0.05, **P<0.01, and ***P<0.001. Statistical analyses were performed using T-test. DN, double-negative;

FIG. 22 demonstrates the effect of glutamine blockade on kidney T cell metabolism in ischemic AKI. JHU083 treatment upregulated hexokinase II and pS6 in T cells from post-ischemic kidneys (n=9 mice in each group). *P<0.05, **P<0.01, and ***P<0.001. Statistical analyses were performed using T-test. DN, double-negative; GLS, glutaminase; pS6, phospho-S6 ribosomal protein; and

FIG. 23 demonstrates the effects of glutamine blockade on T cell phenotypes in normal steady state kidneys. JHU083 treatment reduced CD44 expression and increased CD62L expression on CD4⁺ and CD8 T⁺ cells in normal kidneys, indicating glutamine blockade reduced effector-memory phenotypes. CD69 expression was reduced in CD8⁺ T cells from the JHU083 treated mice. Ki67 expression was reduced in CD4⁺ and CD8⁺ T cells from JHU083 treated normal kidneys. *P<0.05, **P<0.01, and ***P<0.001. Statistical analyses were performed using T-test. DN, double-negative.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Methods for Preventing and Treating Acute Kidney Injury

The presently disclosed subject matter demonstrates that the compound referred to herein as JHU083, and related compounds, target T cell metabolism and glutamine in a mouse model of acute kidney injury. It was found that pre-treatment of mice undergoing ischemic AKI with JHU083 attenuates the worsening kidney function. This discovery sets the stage for using JHU083 and related compounds for preventing, and possibly treating AKI, as well.

Acute kidney injury (AKI), also referred to as acute renal failure (ARF), is defined as an abrupt (e.g., within 48 hours or less) decrease in kidney function, which encompasses both injury (structural damage) and impairment (loss of function). AKI has a high mortality and morbidity for which there is no specific therapy except supportive care. Even when injured kidneys are dialyzed, a large number of patients die and ischemic acute kidney injury remains a major diagnostic and therapeutic problem in native kidneys and allografts.

Classification of AKI includes pre-renal AKI, acute post-renal obstructive nephropathy and intrinsic acute kidney diseases. Of these, only intrinsic AKI represents true kidney disease, while pre-renal and post-renal AKI are the consequence of extra-renal diseases leading to the decreased glomerular filtration rate (GFR). If these pre- and/or post-renal conditions persist, they will eventually evolve to renal cellular damage and hence intrinsic renal disease. The current diagnostic approach of AKI is based on an acute decrease of GFR, as reflected by an acute rise in sCr levels and/or a decline in UO over a given time interval.

Acute kidney injury can have many different causes including:
(i) ischemia, i.e., decreased blood flow to one or more organs, which can be caused by low blood pressure (i.e., “hypotension”) or shock, blood or fluid loss (such as bleeding, severe diarrhea), heart attack, heart failure, and other conditions leading to decreased heart function, organ failure (e.g., heart or liver failure), overuse of pain medicines called “NSAIDs”, which are used to reduce swelling or relieve pain from headaches, colds, flu, and other ailments, including ibuprofen, ketoprofen, and naproxen, severe allergic reactions, burns, injury, and surgery, including transplants;
(ii) direct damage or injury to the kidneys, including direct damage to the kidneys by a nephrotoxin, such as a chemotherapeutic agent, such as an antineoplastic agent, including cisplatin or methotrexate, a contrast agent, or a nephrotoxic medication, including, but not limited to, certain antibiotics, such as penicillins, cephalosporins, and quinolones, NSAIDS, gold preparations, thiazides, sulfonamides, aminoglycosides, ACE inhibitors, angiotensin II antagonists, antivirals, such as foscarnet, and other nephrotoxic medications including, but not limited to, vancomycin, ranitidine, amphotericin B, and the like, or from a disease or condition, such a sepsis, a cancer, such as multiple myeloma, vasculitis, interstitial nephritis, scleroderma, and conditions that cause inflammation or damage to the kidney tubules, to the small blood vessels in the kidneys, or to the filtering units in the kidneys, such as tubular necrosis, glomerulonephritis, vasculitis, and thrombotic microangiopathy; and
(iii) blockage of the urinary tract, which can be caused by bladder, prostate, or cervical cancer or an enlarged prostate, problems with the nervous system that affect the bladder and urination, kidney stones, and blood clots in the urinary tract.

Glutamine antagonists, such as 6-diazo-5-oxo-L-norleucine (DON), azaserine, and acivicin, have been shown to have anti-cancer activities in multiple preclinical and clinical studies. The toxicity of such glutamine antagonists at doses necessary for their anticancer efficacy, however, has hampered their clinical development for cancer and other indications. The presently disclosed subject matter provides the use of glutamine antagonists, or prodrugs and analogs thereof, at doses less than that used for their anticancer efficacy, for preventing and treating acute kidney injury (AKI).

In one embodiment, the presently disclosed subject matter provides a method for preventing or treating a subject afflicted with, suspected of having, or susceptible to having an acute kidney injury, the method comprising administering to the subject at least one glutamine antagonist, or prodrug or analog thereof, in an amount effective to prevent or treat the acute kidney injury.

As used herein, the term “glutamine antagonist” refers to an agent that blocks or interferes with the synthesis or use of glutamine in a cell, and preferably in a cell that is part of a living organism. When it is said that the glutamine antagonist interferes with the synthesis of glutamine, it is meant that the antagonist acts to reduce the amount or rate of glutamine synthesis to less than the amount or rate that would be experienced in the absence of the glutamine antagonist. When it is said that the glutamine antagonist interferes with the use of glutamine, it is meant that the antagonist acts to inhibit or block a metabolic pathway downstream of glutamine, that is, a pathway in which glutamine acts as a precursor of one or more non-glutamine compounds, or that the antagonist acts to deplete glutamine in a cell or an organism by reacting the glutamine to form a non-glutamine product, or by reversibly or irreversibly binding with glutamine to reduce its availability.

In some embodiments, a glutamine antagonist is a compound that inhibits the synthesis of glutamine. Examples of compounds having this activity include inhibitors of glutamine synthase (EC 6.3.1.2), such as L-methionine-DL-sulfoximine, and phosphinothricin; inhibitors of glutamate synthase (EC 1.4.1.13); inhibitors of amidophosphoribosyltransferase (EC 2.4.2.14); and inhibitors of glutamate dehydrogenase; and mixtures of any two or more of these.

In some embodiments, a glutamine antagonist is a glutamine depleting enzyme. Examples of such enzymes include carbamoyl-phosphate synthase (EC 6.3.5.5), glutamine-pyruvate transaminase (EC 2.6.1.15), glutamine-tRNA ligase (EC 6.1.1.18), glutaminase (EC 3.5.1.2), D-glutaminase (EC 3.5.1.35), glutamine N-acyltransferase (EC2.3.1.68), glutaminase-asparaginase (in particular glutaminase-asparaginase of Pseudomonas 7a and Acinatobacter sp.), and mixtures of any two or more of these.

In some embodiments, a glutamine antagonist is a compound that reacts with glutamine under intracellular conditions to form a non-glutamine product. An example of a compound having this property is phenylbutyrate (see Darmaun et al., Phenylbutyrate-induce glutamine depletion in humans: effect on leucine metabolism, pp. E801-E807, in Glutamine Depletion and Protein Catabolism, Am. Physiol. Soc. (1998)). Another example of a glutamine antagonist having this characteristic is phenylacetate (see, U.S. Pat. No. 6,362,226), which is incorporated herein by reference in its entirety.

In some embodiments, a glutamine antagonist is a compound that inhibits glutamine uptake by cells. Examples of compounds having this property include alpha-methylaminoisobutyric acid (inhibits GynT plasma membrane glutamine transporter; see, Varoqui et al., J. Biol. Chem., 275(6):4049-4054 (2000), wortmannin, and LY-294002 (inhibits hepatic glutamine transporter; see, Pawlik et al., Am. J. Physiol. Gastrointest. Liver Physiol., 278:G532-G541 (2000)).

In some embodiments, a glutamine antagonist is a glutamine binding compound that reduces the biological availability of glutamine.

In some embodiments, a glutamine antagonist is a glutamine analog that interferes with a glutamine metabolic pathway. Examples of compounds that can act in this manner include acivicin (L-(alpha S,5S)-alpha-amino-3-chloro-4,5-dihydro-5-isoxazoleacetic acid), DON (6-diazo-5-oxo-L-norleucine), azaserine, azotomycin, chloroketone (L-2-amino-4-oxo-5-chloropentanoic acid), N³-(4-methoxyfumaroyl)-L-2,3-diaminopropanoic acid (FMDP) (inactivates glucosamine-6-phosphate synthase (EC 2.6.1.16), see, Zgódka et al., Microbiology, 147:1955-1959 (2001)), (3S,4R)-3,4-dimethyl-L-glutamine, (3S,4R)-3,4-dimethyl-L-pyroglutamic acid (see, Acevedo et al., Tetrahedron., 57:6353-6359 (2001)), 1,5-N,N′-disubstituted-2-(substituted benzenesulphonyl) glutamamides (see, Srikanth et al., Bioorganic and Medicinal Chemistry, (2002)), or a mixture of any two or more of these. In some embodiments, at least one glutamine antagonist is a glutamine analog. In some embodiments, at least one glutamine antagonist is selected from the group consisting of acivicin (L-(alpha S, 5S)-alpha-amino-3-chloro-4,5-dihydro-5-isoxazoleacetic acid), azaserine, and 6-diazo-5-oxo-norleucine (DON).

In some embodiments, at least one glutamine antagonist is a prodrug of a glutamine analog. In some embodiments, at least one glutamine antagonist is a prodrug of acivicin (L-(alpha S, 5S)-alpha-amino-3-chloro-4,5-dihydro-5-isoxazoleacetic acid), azaserine, and 6-diazo-5-oxo-norleucine (DON).

Suitable prodrugs of glutamine analogs are disclosed in U.S. Pat. No. 10,738,066 for Prodrugs of Glutamine Analogs to Slusher et al., issued Aug. 11, 2020, which is incorporated herein by reference in its entirety.

In some embodiments, a prodrug of a glutamine antagonist, or a pharmaceutically acceptable salt or ester thereof has a structure of formula (I):

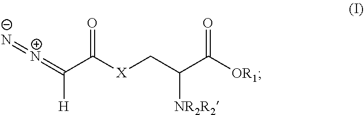

wherein: X is selected from the group consisting of a bond, —O—, and —(CH₂)ₙ—, wherein n is an integer selected from the group consisting of 1, 2, 3, 4, 5, 6, 7, and 8; R₁ is selected from the group consisting of H and a first prodrug-forming moiety capable of forming a salt or an ester; and R₂ is H or a second prodrug-forming moiety capable of forming an amide linkage, a carbamate linkage, a phosphoramidate linkage or a phosphorodiamidate linkage with the nitrogen adjacent to R₂; R₂′ is selected from the group consisting of H, C₁-C₆ alkyl, substituted C₁-C₆ alkyl, or R₂ and R₂′ together form a ring structure comprising —C(═O)-G-C(═O)—, wherein G is selected from the group consisting of C₁-C₈ alkylene, C₁-C₈ heteroalkylene, C₅-C₈ cycloalkylene, C₆-C₁₂ arylene, C₅-C₁₄ heteroarylene, bivalent C₄-C₁₀ heterocycle, each of which can be optionally substituted; or R₁ and R₂′ together form a 4- to 6-membered heterocylic ring comprising the oxygen atom adjacent to R₁ and the nitrogen atom adjacent to R₂′; provided that the compound has at least one prodrug-forming moiety selected from the group consisting of the first and the second prodrug-forming moieties.

As used herein, the term “amide linkage” comprises a structure represented by the formula:

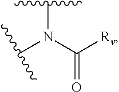

wherein Rᵥ is selected from the group consisting of alkyl, substituted alkyl, cycloalkyl, substituted cycloalkyl, aryl, substituted aryl, aralkyl, substituted aralkyl, heterocyclyl, substituted heterocyclyl, alkenyl, substituted alkenyl, cycloalkenyl, substituted cycloalkenyl, alkylamine, substituted alkylamine, heteroaryl, and substituted heteroaryl.

As used herein, the term “carbamate linkage” comprises a structure represented by the formula:

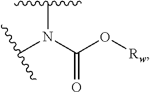

wherein R_{w} is selected from the group consisting of alkyl, substituted alkyl, cycloalkyl, substituted cycloalkyl, aryl, substituted aryl, aralkyl, substituted aralkyl, heterocyclyl, substituted heterocyclyl, alkenyl, substituted alkenyl, cycloalkenyl, substituted cycloalkenyl, alkylamine, substituted alkylamine, heteroaryl, and substituted heteroaryl.

As used herein, the term “phosphoramidate linkage” comprises a structure represented by the formula:

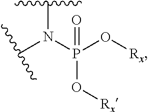

wherein Rₓ and Rₓ′ are each independently selected from the group consisting of alkyl, substituted alkyl, cycloalkyl, substituted cycloalkyl, aryl, substituted aryl, aralkyl, substituted aralkyl, heterocyclyl, substituted heterocyclyl, alkenyl, substituted alkenyl, cycloalkenyl, substituted cycloalkenyl, alkylamine, substituted alkylamine, heteroaryl, and substituted heteroaryl.

As used herein, the term “phosphorodiamidate linkage” comprises a structure represented by the formula:

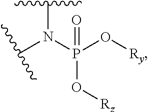

wherein R_{y} and R_{z} are each independently selected from the group consisting of H, alkyl, substituted alkyl, cycloalkyl, substituted cycloalkyl, heterocyclyl, substituted heterocyclyl, alkenyl, substituted alkenyl, cycloalkenyl, substituted cycloalkenyl, —(CR₃R₄)ₘ—Z, —(CR₃R₄)ₘ-Q-Z, aryl, substituted aryl, alkylamine, substituted alkylamine, heteroaryl, substituted heteroaryl, and

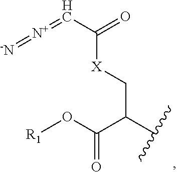

In some embodiments, X is —CH₂—, and n is 1.

In other embodiments, X is —O—. In some embodiments, the prodrug compound has both the first prodrug-forming moiety and the second prodrug-forming moiety. In some embodiments, the glutamine analog is a glutamine antagonist, i.e., the prodrug is a prodrug of a glutamine analog that antagonizes a glutamine pathway. Exemplary glutamine antagonists include, without limitation, 6-diazo-5-oxo-norleucine (DON), and aza-serine, and 5-diazo-4-oxo-L-norvaline (L-DONV).

In some embodiments, the presently disclosed subject matter provides a prodrug of DON. In some embodiments, the prodrug of DON has a structure of formula (I). In some embodiments, the presently disclosed subject matter provides a prodrug of L-DONV. In some embodiments, the prodrug of L-DONV has a structure of formula (I). In some embodiments, the presently disclosed subject matter provides a prodrug of azaserine. In some embodiments, the prodrug of azaserine has a structure of formula (I).

In some embodiments, R₁ of formula (I) comprises a residue PRO₁ of the prodrug-forming moiety, which, together with a basic moiety and the terminal hydroxyl group forms a salt.

In some embodiments, R₁ of formula (I) comprises a residue PRO₁ of the prodrug-forming moiety, which, together with an alkyl group and the oxygen of an adjoining hydroxyl group forms an ester.

In some embodiments, R₁ of formula (I) comprises a residue PRO₁ of the prodrug-forming moiety, which, together with an alkyl group and the nitrogen adjoining the R₂′ group, forms an azlactone or an oxazolidone.

In some embodiments, R₁ of formula (I) is selected from the group consisting of H, alkyl, substituted alkyl, cycloalkyl, substituted cycloalkyl, alkenyl, substituted alkenyl, alkynyl, substituted alkynyl, cycloalkenyl, substituted cycloalkenyl, tri(hydrocarbyl)ammonium, and tetra(hydrocarbyl)ammonium. Preferred alkyl group, cycloalkyl group, alkenyl group, alkynyl group, and cycloalkenyl group substituents include alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl.

In some embodiments, R₁ of formula (I) is not H. In some embodiments, R₁ of formula (I) is not H when R₂ and R₂′ are H. In some embodiments, R₂ and R₂′ of formula (I) are each H when and R₁ is not H.

In some embodiments, R₁ of formula (I) is selected from the group consisting of a C₁₋₆ straight-chain alkyl, a substituted C₁₋₆ straight-chain alkyl, a C₁₋₆ branched alkyl, a substituted C₁₋₆ branched alkyl, tri(C₁-C₈-alkyl)ammonium, tetra(C₁-C₈-alkyl)ammonium, triphenylammonium, tri(hydroxy-C₁-C₈-alkyl)ammonium, and tetra(hydroxy-C₁-C₈-alkyl)ammonium.

In some embodiments, R₁ of formula (I) is selected from the group consisting of methyl, ethyl, isopropyl, cyclopentyl, cyclohexyl, trimethylammonium, triethylammonium, tri(hydroxyethyl)ammonium, tripropylammonium, and tri(hydroxypropyl)ammonium. In some embodiments, R₁ of formula (I) is methyl. In some embodiments, R₁ of formula (I) is ethyl. In some embodiments, R₁ of formula (I) is isopropyl.

In some embodiments, R₂ of formula (I) comprises a residue PRO₂ of the second prodrug-forming moiety, which, together with a carbonyl, oxy carbonyl, or phosphonyl group and the nitrogen of the adjoining NH, forms an amide, a carbamate, phosphoramidate, or phosphorodiamidate linkage.

In some embodiments, R₂ of formula (I) comprises a moiety selected from the group consisting of an amino acid, an N-substituted amino acid, a peptide, a substituted peptide, a monocyclic ring, a substituted monocyclic ring, a bicyclic ring, a substituted bicyclic ring, a purine nucleoside, a substituted purine nucleoside, a pyrimidine nucleoside, and a substituted pyrimidine nucleoside.

In some aspects, a prodrug of a glutamine antagonist, or a pharmaceutically acceptable salt or ester thereof has a structure of formula (I):

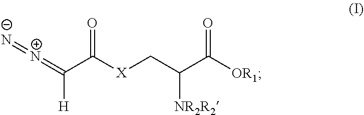

wherein: X is selected from the group consisting of a bond, —O—, and —(CH₂)ₙ—, wherein n is an integer selected from the group consisting of 1, 2, 3, 4, 5, 6, 7, and 8; R₁ is selected from the group consisting of H and a first prodrug-forming moiety capable of forming a salt or an ester; and R₂ is H or a second prodrug-forming moiety capable of forming an amide linkage, a carbamate linkage, a phosphoramidate linkage or a phosphorodiamidate linkage with the nitrogen adjacent to R₂; R₂′ is selected from the group consisting of H, C₁-C₆ alkyl, substituted C₁-C₆ alkyl, or R₂ and R₂′ together form a ring structure comprising —C(═O)-G-C(═O)—, wherein G is selected from the group consisting of C₁-C₈ alkylene, C₁-C₈ heteroalkylene, C₅-C₈ cycloalkylene, C₆-C₁₂ arylene, C₅-C₁₄ heteroarylene, bivalent C₄-C₁₀ heterocycle, each of which can be optionally substituted; or R₁ and R₂′ together form a 4- to 6-membered heterocylic ring comprising the oxygen atom adjacent to R₁ and the nitrogen atom adjacent to R₂′; provided that the compound has at least one prodrug-forming moiety selected from the group consisting of the first and the second prodrug-forming moieties.

As used herein, the term “amide linkage” comprises a structure represented by the formula:

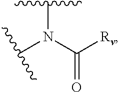

wherein Rᵥ is selected from the group consisting of alkyl, substituted alkyl, cycloalkyl, substituted cycloalkyl, aryl, substituted aryl, aralkyl, substituted aralkyl, heterocyclyl, substituted heterocyclyl, alkenyl, substituted alkenyl, cycloalkenyl, substituted cycloalkenyl, alkylamine, substituted alkylamine, heteroaryl, and substituted heteroaryl.

As used herein, the term “carbamate linkage” comprises a structure represented by the formula:

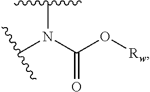

wherein R_{w} is selected from the group consisting of alkyl, substituted alkyl, cycloalkyl, substituted cycloalkyl, aryl, substituted aryl, aralkyl, substituted aralkyl, heterocyclyl, substituted heterocyclyl, alkenyl, substituted alkenyl, cycloalkenyl, substituted cycloalkenyl, alkylamine, substituted alkylamine, heteroaryl, and substituted heteroaryl.

As used herein, the term “phosphoramidate linkage” comprises a structure represented by the formula:

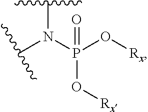

wherein Rₓ and Rₓ′ are each independently selected from the group consisting of alkyl, substituted alkyl, cycloalkyl, substituted cycloalkyl, aryl, substituted aryl, aralkyl, substituted aralkyl, heterocyclyl, substituted heterocyclyl, alkenyl, substituted alkenyl, cycloalkenyl, substituted cycloalkenyl, alkylamine, substituted alkylamine, heteroaryl, and substituted heteroaryl.

As used herein, the term “phosphorodiamidate linkage” comprises a structure represented by the formula:

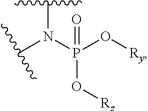

wherein R_{y} and R_{z} are each independently selected from the group consisting of H, alkyl, substituted alkyl, cycloalkyl, substituted cycloalkyl, heterocyclyl, substituted heterocyclyl, alkenyl, substituted alkenyl, cycloalkenyl, substituted cycloalkenyl, —(CR₃R₄)ₘ—Z, —(CR₃R₄)ₘ-Q-Z, aryl, substituted aryl, alkylamine, substituted alkylamine, heteroaryl, substituted heteroaryl, and

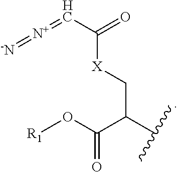

In some embodiments, X is —CH₂—, and n is 1.

In other embodiments, X is —O—. In some embodiments, the prodrug compound has both the first prodrug-forming moiety and the second prodrug-forming moiety. In some embodiments, the glutamine analog is a glutamine antagonist, i.e., the prodrug is a prodrug of a glutamine analog that antagonizes a glutamine pathway. Exemplary glutamine antagonists include, without limitation, 6-diazo-5-oxo-norleucine (DON), and aza-serine, and 5-diazo-4-oxo-L-norvaline (L-DONV).

In some embodiments, the presently disclosed subject matter provides a prodrug of DON. In some embodiments, the prodrug of DON has a structure of formula (I). In some embodiments, the presently disclosed subject matter provides a prodrug of L-DONV. In some embodiments, the prodrug of L-DONV has a structure of formula (I). In some embodiments, the presently disclosed subject matter provides a prodrug of azaserine. In some embodiments, the prodrug of azaserine has a structure of formula (I).

In some embodiments, R₁ of formula (I) comprises a residue PRO₁ of the prodrug-forming moiety, which, together with a basic moiety and the terminal hydroxyl group forms a salt.

In some embodiments, R₁ of formula (I) comprises a residue PRO₁ of the prodrug-forming moiety, which, together with an alkyl group and the oxygen of an adjoining hydroxyl group forms an ester.

In some embodiments, R₁ of formula (I) comprises a residue PRO₁ of the prodrug-forming moiety, which, together with an alkyl group and the nitrogen adjoining the R₂′ group, forms an azlactone or an oxazolidone.

In some embodiments, R₁ of formula (I) is selected from the group consisting of H, alkyl, substituted alkyl, cycloalkyl, substituted cycloalkyl, alkenyl, substituted alkenyl, alkynyl, substituted alkynyl, cycloalkenyl, substituted cycloalkenyl, tri(hydrocarbyl)ammonium, and tetra(hydrocarbyl)ammonium. Preferred alkyl group, cycloalkyl group, alkenyl group, alkynyl group, and cycloalkenyl group substituents include alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl.

In some embodiments, R₁ of formula (I) is not H. In some embodiments, R₁ of formula (I) is not H when R₂ and R₂′ are H. In some embodiments, R₂ and R₂′ of formula (I) are each H when and R₁ is not H.

In some embodiments, R₁ of formula (I) is selected from the group consisting of a C₁₋₆ straight-chain alkyl, a substituted C₁₋₆ straight-chain alkyl, a C₁₋₆ branched alkyl, a substituted C₁₋₆ branched alkyl, tri(C₁-C₈-alkyl)ammonium, tetra(C₁-C₈-alkyl)ammonium, triphenylammonium, tri(hydroxy-C₁-C₈-alkyl)ammonium, and tetra(hydroxy-C₁-C₈-alkyl)ammonium.

In some embodiments, R₁ of formula (I) is selected from the group consisting of methyl, ethyl, isopropyl, cyclopentyl, cyclohexyl, trimethylammonium, triethylammonium, tri(hydroxyethyl)ammonium, tripropylammonium, and tri(hydroxypropyl)ammonium. In some embodiments, R₁ of formula (I) is methyl. In some embodiments, R₁ of formula (I) is ethyl. In some embodiments, R₁ of formula (I) is isopropyl.

In some embodiments, R₂ of formula (I) comprises a residue PRO₂ of the second prodrug-forming moiety, which, together with a carbonyl, oxy carbonyl, or phosphonyl group and the nitrogen of the adjoining NH, forms an amide, a carbamate, phosphoramidate, or phosphorodiamidate linkage.

In some embodiments, R₂ of formula (I) comprises a moiety selected from the group consisting of an amino acid, an N-substituted amino acid, a peptide, a substituted peptide, a monocyclic ring, a substituted monocyclic ring, a bicyclic ring, a substituted bicyclic ring, a purine nucleoside, a substituted purine nucleoside, a pyrimidine nucleoside, and a substituted pyrimidine nucleoside.

In some embodiments, R₂ of formula (I) is selected from the group consisting of H, alkyl, —C(═O)—Ar, —C(═O)—Y—(CR₃R₄)ₘ—Ar, —C(═O)—Y—(CR₃R₄)ₘ—NR₅R₆, —P(═O)(OR₇)ₙ(NHR₉)ₒ, —C(═O)—Y—(CR₃R₄)ₘ—Ar—O—C(═O)—R₈, —C(═O)—Y—(CR₃R₄)ₘ—Ar—O—R₈, —C(═O)—O—(CR₃R₄)ₘ—O—C(═O)—R₁₀, —C(═O)—O—R₉, —C(═O)—Y—(CR₃R₄)ₘ—Ar—O—C(═O)—Ar, and —C(═O)—Y—(CR₃R₄)ₘ—Ar—NR₅R₆; wherein: Y is —O— or a bond; m is an integer selected from the group consisting of 0, 1, 2, 3, 4, 5, 6, 7, and 8; each n and o is an integer from 0 to 2 provided that the sum of n and o is 2; R₃ and R₄ is independently H, C₁-C₆ alkyl or substituted C₁-C₆ alkyl, aryl or substituted aryl, —(CR₃R₄)ₘ—NR₅R₆, or

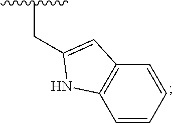 [SMILES: *Cc1cc2ccccc2[nH]1]

each R₅ and R₆ is independently H, alkyl, —C(═O)—(CR₃R₄)ₘ, —C(═O)—(NR₅R₆), or —C(═O)—(CR₃R₄)ₘ—NR₅R₆; each R₇ is independently selected from the group consisting of H, alkyl, substituted alkyl, cycloalkyl, substituted cycloalkyl, heterocyclyl, substituted heterocyclyl, alkenyl, substituted alkenyl, cycloalkenyl, substituted cycloalkenyl, —(CR₃R₄)ₘ—Z, —(CR₃R₄)ₘ-Q-Z, wherein Q is a monosaccharide, aryl, substituted aryl, heteroaryl, substituted heteroaryl, and wherein Z is

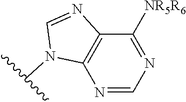

or wherein R₇ together with the oxygen atom to which it is attached forms a purine or pyrimidine nucleoside; each R₉ is independently selected from the group consisting of H, alkyl, substituted alkyl, cycloalkyl, substituted cycloalkyl, heterocyclyl, substituted heterocyclyl, alkenyl, substituted alkenyl, cycloalkenyl, substituted cycloalkenyl, —(CR₃R₄)ₘ—Z, aryl, substituted aryl, heteroaryl, substituted heteroaryl, and

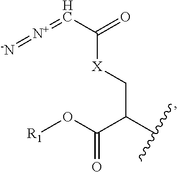

wherein R₁ and X are as defined above, provided that R₁ is not H; each R₈ is independently alkyl, substituted alkyl, cycloalkyl, substituted cycloalkyl, monosaccharide, acylated monosaccharide, aryl, substituted aryl, heteroaryl, substituted heteroaryl; each R₁₀ is independently alkyl, substituted alkyl, cycloalkyl, substituted cycloalkyl, monosaccharide, acylated monosaccharide, aryl, substituted aryl, heteroaryl, substituted heteroaryl; and Ar is aryl, substituted aryl, heteroaryl, or substituted heteroaryl. It should be appreciated that in addition to substitutions on the amino group of Z, one or more substitutions R₃, R₄, R₅, and/or R₆ can be made to the 5 or 6 membered rings of Z.

Structures of representative DON prodrugs are provided in Table 1.

TABLE 1
Structures of Representative DON Prodrugs

| IOCB No./ | | |
|---|---|---|
| Compound No. | Structure | MW |
| Compound 1 (DON) | 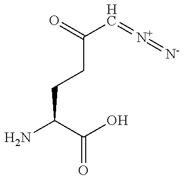 [SMILES: [N-]=[N+]=CC(=O)CC[C@H](N)C(=O)O] | 171.15 |
| Compound 3 | 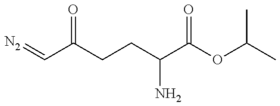 | 213.24 |
| Compound 4 | 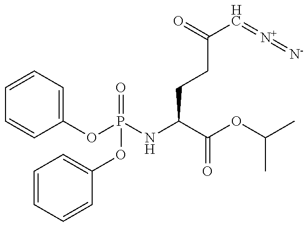 [SMILES: CC(C)OC(=O)[C@H](CCC(=O)C=[N+]=[N-])NP(=O)(Oc1ccccc1)Oc1ccccc1] | 445.41 |
| Compound 6 | 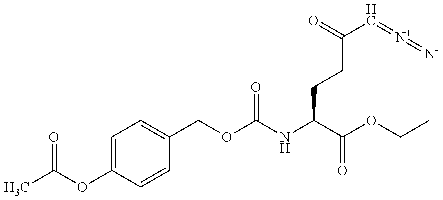 [SMILES: CCOC(=O)[C@H](CCC(=O)C=[N+]=[N-])NC(=O)OCc1ccc(OC(C)=O)cc1] | 391.38 |
| Compound 7 | 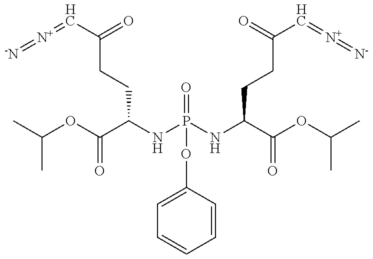 | 564.53 |
| Compound 9 | 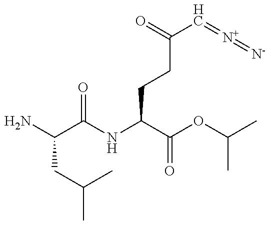 [SMILES: CC(C)C[C@H](N)C(=O)N[C@@H](CCC(=O)C=[N+]=[N-])C(=O)OC(C)C] | 326.39 |
| Compound 11 | 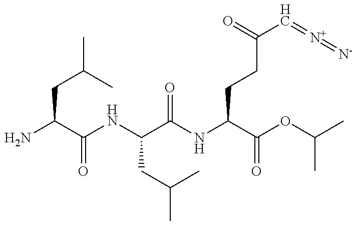 [SMILES: CC(C)C[C@H](NC(=O)[C@@H](N)CC(C)C)C(=O)N[C@@H](CCC(=O)C=[N+]=[N-])C(=O)OC(C)C] | 439.55 |
| Compound 13 | 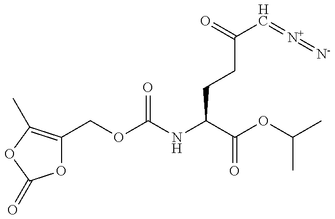 [SMILES: Cc1oc(=O)oc1COC(=O)N[C@@H](CCC(=O)C=[N+]=[N-])C(=O)OC(C)C] | 369.18 |
| Compound 14a | 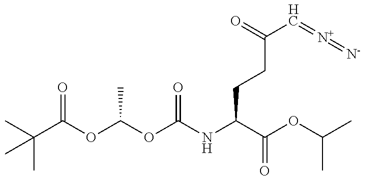 [SMILES: CC(C)OC(=O)[C@H](CCC(=O)C=[N+]=[N-])NC(=O)O[C@@H](C)OC(=O)C(C)(C)C] | 385.41 |
| Compound 14b | 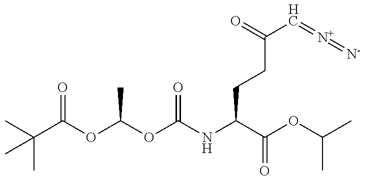 [SMILES: CC(C)OC(=O)[C@H](CCC(=O)C=[N+]=[N-])NC(=O)O[C@H](C)OC(=O)C(C)(C)C] | |
| Compound 15 | 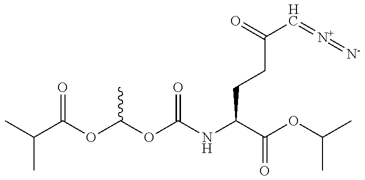 [SMILES: CC(C)OC(=O)[C@H](CCC(=O)C=[N+]=[N-])NC(=O)OC(C)OC(=O)C(C)C] | 371.39 |
| Compound 17 | 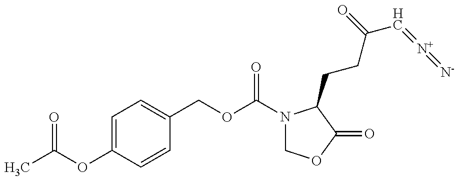 [SMILES: CC(=O)Oc1ccc(COC(=O)N2COC(=O)[C@@H]2CCC(=O)C=[N+]=[N-])cc1] | 375.33 |
| Compound 20 | 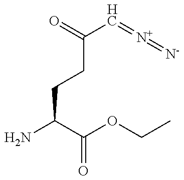 | 199.21 |
| Compound 22 | 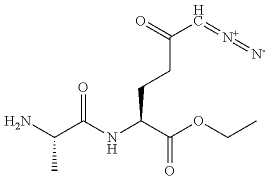 [SMILES: CCOC(=O)[C@H](CCC(=O)C=[N+]=[N-])NC(=O)[C@H](C)N] | 270.28 |
| Compound 23 | 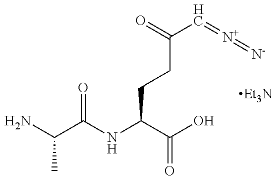 [SMILES: "·Et3N" ; C[C@H](N)C(=O)N[C@@H](CCC(=O)C=[N+]=[N-])C(=O)O] | 343.42 |
| Compound 25 | 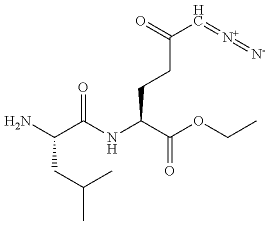 [SMILES: CCOC(=O)[C@H](CCC(=O)C=[N+]=[N-])NC(=O)[C@@H](N)CC(C)C] | 312.36 |
| Compound 26 | 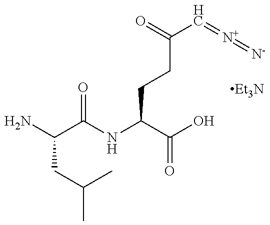 [SMILES: "·Et3N" ; CC(C)C[C@H](N)C(=O)N[C@@H](CCC(=O)C=[N+]=[N-])C(=O)O] | 385.50 |
| Compound 28 | 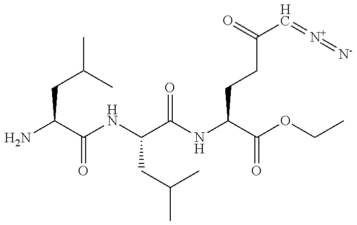 [SMILES: CCOC(=O)[C@H](CCC(=O)C=[N+]=[N-])NC(=O)[C@H](CC(C)C)NC(=O)[C@@H](N)CC(C)C] | 425.52 |
| Compound 29 | 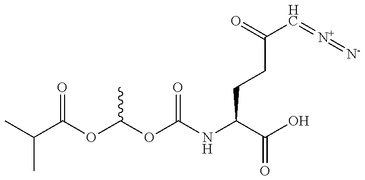 [SMILES: CC(OC(=O)N[C@@H](CCC(=O)C=[N+]=[N-])C(=O)O)OC(=O)C(C)C] | 329.31 |
| Compound 30 | 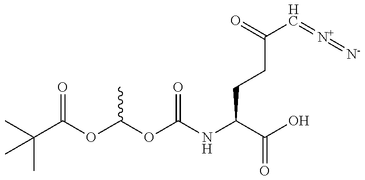 [SMILES: CC(OC(=O)N[C@@H](CCC(=O)C=[N+]=[N-])C(=O)O)OC(=O)C(C)(C)C] | 343.33 |
| Compound 31 | 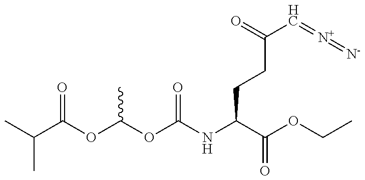 [SMILES: CCOC(=O)[C@H](CCC(=O)C=[N+]=[N-])NC(=O)OC(C)OC(=O)C(C)C] | 357.37 |
| Compound 32 | 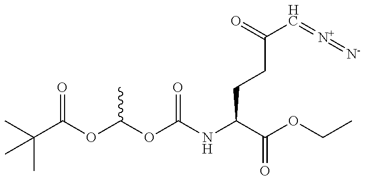 [SMILES: CCOC(=O)[C@H](CCC(=O)C=[N+]=[N-])NC(=O)OC(C)OC(=O)C(C)(C)C] | 371.39 |
| Compound 34 | 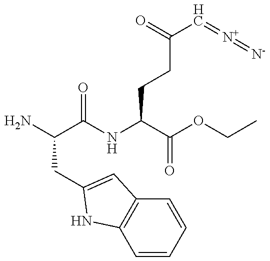 [SMILES: CCOC(=O)[C@H](CCC(=O)C=[N+]=[N-])NC(=O)[C@@H](N)Cc1cc2ccccc2[nH]1] | 385.42 |
| Compound 35 | 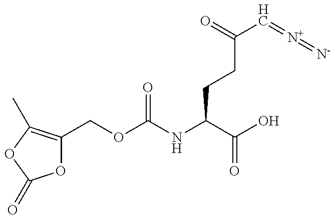 [SMILES: Cc1oc(=O)oc1COC(=O)N[C@@H](CCC(=O)C=[N+]=[N-])C(=O)O] | 327.25 |
| Compound 36 | 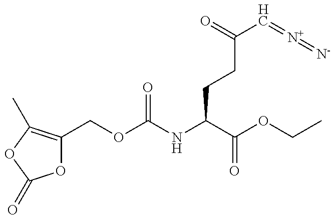 [SMILES: CCOC(=O)[C@H](CCC(=O)C=[N+]=[N-])NC(=O)OCc1oc(=O)oc1C] | 355.30 |
| Compound 38a | 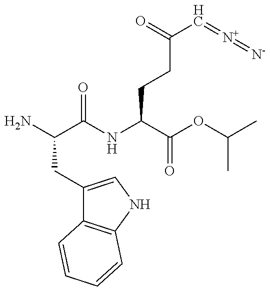 [SMILES: CC(C)OC(=O)[C@H](CCC(=O)C=[N+]=[N-])NC(=O)[C@@H](N)Cc1c[nH]c2ccccc12] | 399.45 |
| Compound 38 | 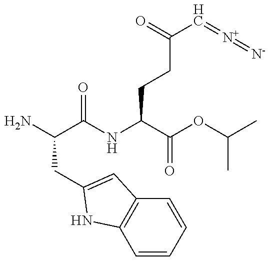 [SMILES: CC(C)OC(=O)[C@H](CCC(=O)C=[N+]=[N-])NC(=O)[C@@H](N)Cc1cc2ccccc2[nH]1] | 399.45 |
| Compound 40 | 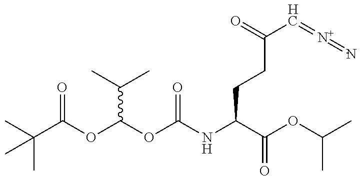 [SMILES: CC(C)OC(=O)[C@H](CCC(=O)C=[N+]=[N-])NC(=O)OC(OC(=O)C(C)(C)C)C(C)C] | 413.47 |
| Compound 42 | 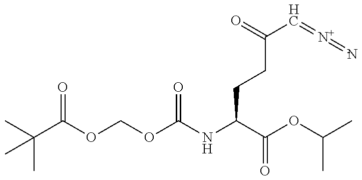 [SMILES: CC(C)OC(=O)[C@H](CCC(=O)C=[N+]=[N-])NC(=O)OCOC(=O)C(C)(C)C] | 371.39 |
| Compound 44 | 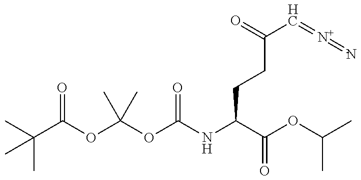 [SMILES: CC(C)OC(=O)[C@H](CCC(=O)C=[N+]=[N-])NC(=O)OC(C)(C)OC(=O)C(C)(C)C] | 2.44 |
| Compound 47 | 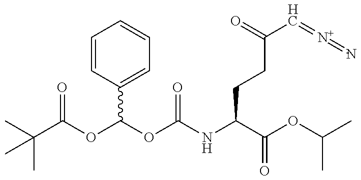 [SMILES: CC(C)OC(=O)[C@H](CCC(=O)C=[N+]=[N-])NC(=O)OC(OC(=O)C(C)(C)C)c1ccccc1] | 447.49 |
| Compound 49 | 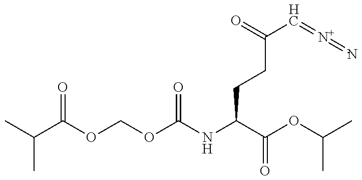 [SMILES: CC(C)OC(=O)[C@H](CCC(=O)C=[N+]=[N-])NC(=O)OCOC(=O)C(C)C] | 357.36 |
| Compound 51 | 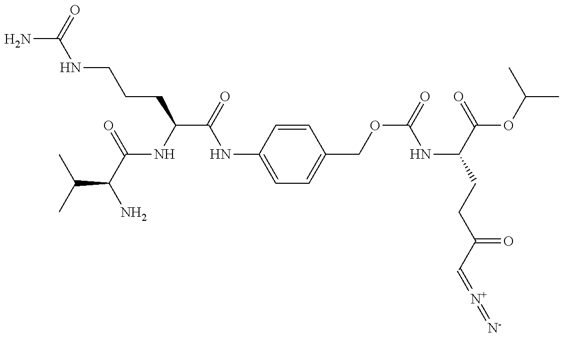 [SMILES: CC(C)OC(=O)[C@H](CCC(=O)C=[N+]=[N-])NC(=O)OCc1ccc(NC(=O)[C@H](CCCNC(N)=O)NC(=O)[C@@H](N)C(C)C)cc1] | 618.69 |
| Compound 52 | 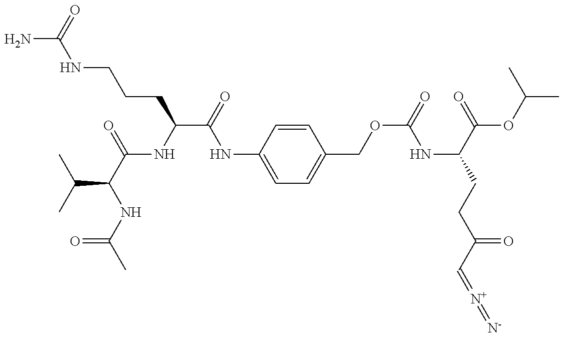 [SMILES: CC(=O)N[C@H](C(=O)N[C@@H](CCCNC(N)=O)C(=O)Nc1ccc(COC(=O)N[C@@H](CCC(=O)C=[N+]=[N-])C(=O)OC(C)C)cc1)C(C)C] | 660.73 |
| Compound 56 | 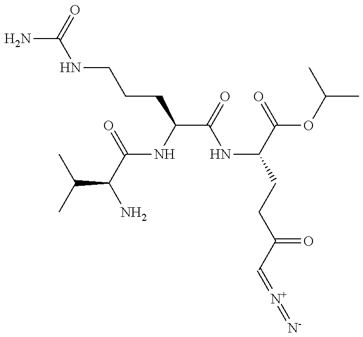 [SMILES: CC(C)OC(=O)[C@H](CCC(=O)C=[N+]=[N-])NC(=O)[C@H](CCCNC(N)=O)NC(=O)[C@@H](N)C(C)C] | 469.54 |
| Compound 57 | 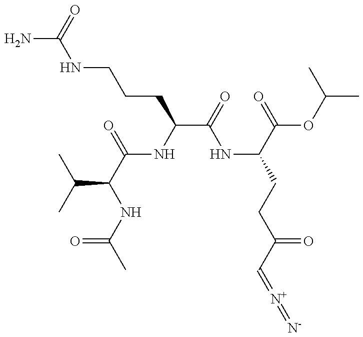 [SMILES: CC(=O)N[C@H](C(=O)N[C@@H](CCCNC(N)=O)C(=O)N[C@@H](CCC(=O)C=[N+]=[N-])C(=O)OC(C)C)C(C)C] | 511.58 |
| Compound 59 | 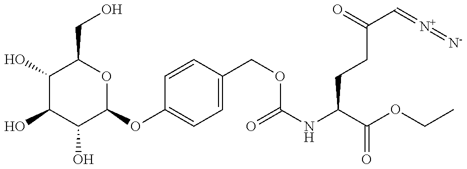 [SMILES: CCOC(=O)[C@H](CCC(=O)C=[N+]=[N-])NC(=O)OCc1ccc(O[C@@H]2O[C@H](CO)[C@@H](O)[C@H](O)[C@H]2O)cc1] | 511.48 |
| Compound 60a | 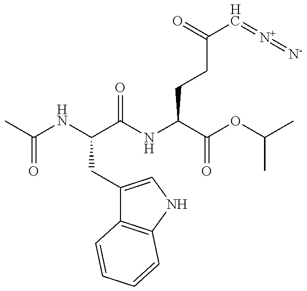 [SMILES: CC(=O)N[C@@H](Cc1c[nH]c2ccccc12)C(=O)N[C@@H](CCC(=O)C=[N+]=[N-])C(=O)OC(C)C] | 464.19 |
| Compound 60 | 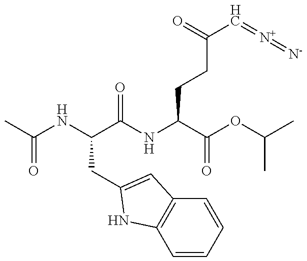 [SMILES: CC(=O)N[C@@H](Cc1cc2ccccc2[nH]1)C(=O)N[C@@H](CCC(=O)C=[N+]=[N-])C(=O)OC(C)C] | 464.19 |
| A | 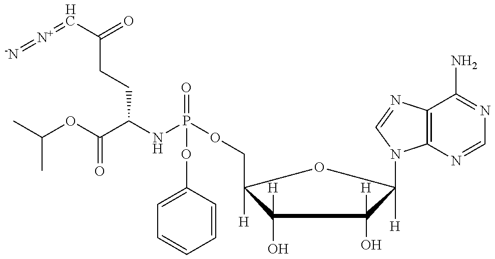 | 618.54 |
| B | 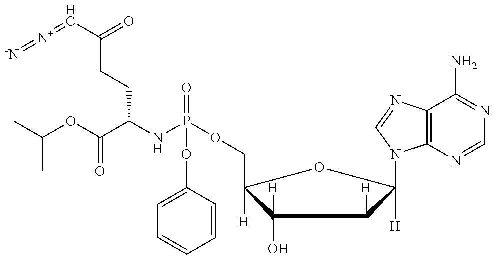 | 602.54 |
| C | 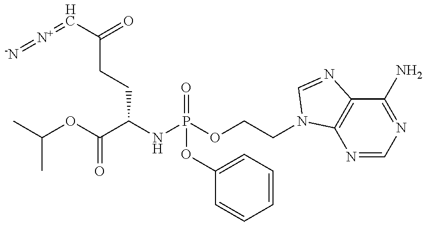 | 530.47 |
| D | 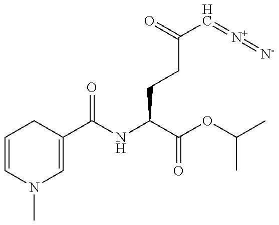 [SMILES: CC(C)OC(=O)[C@H](CCC(=O)C=[N+]=[N-])NC(=O)C1=CN(C)C=CC1] | 334.38 |
| E | 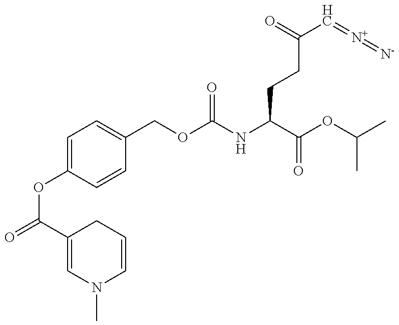 [SMILES: CC(C)OC(=O)[C@H](CCC(=O)C=[N+]=[N-])NC(=O)OCc1ccc(OC(=O)C2=CN(C)C=CC2)cc1] | 484.51 |
| F | 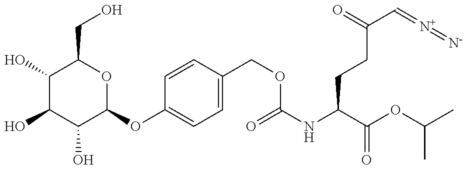 [SMILES: CC(C)OC(=O)[C@H](CCC(=O)C=[N+]=[N-])NC(=O)OCc1ccc(O[C@@H]2O[C@H](CO)[C@@H](O)[C@H](O)[C@H]2O)cc1] | 525.51 |
| G | 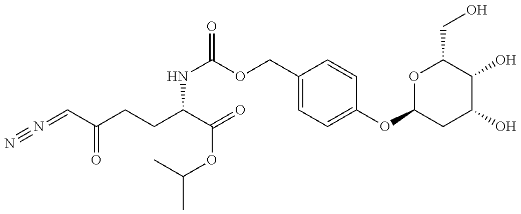 [SMILES: CC(C)OC(=O)[C@H](CCC(=O)C=[N+]=[N-])NC(=O)OCc1ccc(O[C@@H]2C[C@@H](O)[C@@H](O)[C@@H](CO)O2)cc1] | 509.51 |
| LTP 073 | 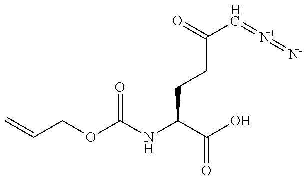 [SMILES: C=CCOC(=O)N[C@@H](CCC(=O)C=[N+]=[N-])C(=O)O] | 255.23 |
| JAM0351 | 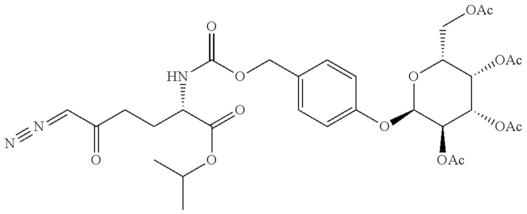 [SMILES: CC(=O)OC[C@H]1O[C@H](Oc2ccc(COC(=O)N[C@@H](CCC(=O)C=[N+]=[N-])C(=O)OC(C)C)cc2)[C@H](OC(C)=O)[C@@H](OC(C)=O)[C@H]1OC(C)=O] | 693.66 |
| JAM0359 | 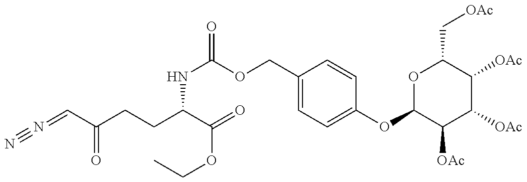 [SMILES: CCOC(=O)[C@H](CCC(=O)C=[N+]=[N-])NC(=O)OCc1ccc(O[C@H]2O[C@H](COC(C)=O)[C@H](OC(C)=O)[C@H](OC(C)=O)[C@H]2OC(C)=O)cc1] | 679.63 |

In general, the presently disclosed methods result in a decrease in the severity of a condition, disease, or disorder (e.g., an acute kidney injury) in a subject. The term “decrease” is meant to inhibit, suppress, attenuate, diminish, arrest, or stabilize a symptom of the condition, disease, or disorder. As used herein, the terms “treat,” “treating,” “treatment,” and the like refer to reducing or ameliorating a disease or condition, and/or symptoms associated therewith. It will be appreciated that, although not precluded, treating a disease or condition does not require that the disorder, condition or symptoms associated therewith be completely eliminated.

The term “preventing” refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

In certain embodiments, the administering of the at least one glutamine antagonist, or a prodrug or analog thereof, modulates a glutamine pathway associated with acute kidney injury. In particular embodiments, the modulation of the glutamine pathway alters T cell metabolism. In more certain embodiments, the modulation of the glutamine pathway blocks glutaminolysis.

In certain embodiments, blockade of glutaminolysis reduces T cell activation and proliferation in post-ischemia reperfusion injury (IRI) kidneys. In certain embodiments, blockade of glutaminolysis reduces CD69 expression in post-ischemia reperfusion injury (IRI) kidney CD8 T cells.

In certain embodiments, the administering of the at least one glutamine antagonist, or a prodrug or analog thereof, causes a decrease in CD44, CD4⁺, and CD8⁺ T cells in post-IRI kidneys.

In certain embodiments, the administering of the at least one glutamine antagonist, or a prodrug or analog thereof, causes a reduced expression of Ki67 cells in post-IRI kidneys.

In certain embodiments, the administering of the at least one glutamine antagonist, or a prodrug or analog thereof, enhances expression of hexokinase II and pS6 on one or more kidney T cells.

In some embodiments, the acute kidney injury is caused by ischemia or an ischemic event, direct injury to the kidney, blockage of a urinary tract, or combinations thereof. In certain embodiments, the direct injury to the kidney is caused by exposure to one or nephrotoxins or from a disease or condition. In particular embodiments, the one or more nephrotoxins include a chemotherapeutic agent, an antibiotic, an NSAID, a gold preparation, a thiazide, a sulfonamide, an aminoglycosides, an ACE inhibitor, an angiotensin II antagonist, an antiviral, vancomycin, ranitidine, amphotericin B, and combinations thereof. In particular embodiments, the disease or condition includes sepsis, a cancer, vasculitis, interstitial nephritis, scleroderma, tubular necrosis, glomerulonephritis, thrombotic microangiopathy, and combinations thereof.

The terms “subject” and “patient” are used interchangeably herein. The subject treated by the presently disclosed methods, uses, glutamine antagonists and compositions comprising those glutamine antagonists in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease.

The terms “decrease,” “reduced,” “reduction,” “decrease,” or “inhibit” are all used herein generally to mean a decrease by a statistically significant amount. However, for avoidance of doubt, “reduced,” “reduction,” “decrease,” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90%, where the decrease is less than 100%. In one embodiment, the decrease includes a 100% decrease (e.g. absent level as compared to a reference sample), or any decrease between 10-100% as compared to a reference level.

The terms “increased,” “increase,” “enhance,” or “activate” are all used herein to generally mean an increase by a statically significant amount; for the avoidance of any doubt, the terms “increased,” “increase,” “enhance,” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

Generally, at least one glutamine antagonist described herein can be used in combination with an additional therapeutic agent (e.g., a pharmaceutically active agent, e.g., a drug approved by a regulatory agency). The therapeutic agent may act synergistically with the glutamine antagonist described herein, or they may independently exert their intended effects. The disclosure contemplates any therapeutic agent which a skilled artisan would use in connection with a method, use, or composition described herein. Examples of therapeutic agents contemplated for use in the presently disclosed methods, uses and compositions in combination with the glutamine antagonists include, but are not limited to, antiviral agents, immunotherapeutic agents, anti-inflammatory agents, neuroprotective agents, neuroregenerative agents, neurotrophic factors, stem and progenitor cells used to replace and/or repair endogenous populations of abnormal, harmful, or unhealthy cells, and vaccines.

Exemplary classes of antiviral agents of use herein include, without limitation, antiviral boosters, antiviral combinations, antiviral interferons, chemokine receptor antagonists, integrase strand transfer inhibitors, NNRTIs, NS5A inhibitors, nucleoside reverse transcriptase inhibitors (NRTIs), protease inhibitors, and purine nucleosides.

Examples of antiviral boosters of use herein include, without limitation, ritonavir, cobicistat, and combinations thereof.

Examples of antiviral combinations of use herein include, without limitation, abacavir and lamivudine (EPZICOM), cobicistat/elvitegravir/emtricitabine/tenofovir (STRIBILD), emtricitabine/tenofovir (TRUVADA), efavirenz/emtricitabine/tenofovir (ATRIPLA), ledipasvir/sofosbuvir (HARVONI), abacavir/lamivudine/zidovudine (TRIZIVIR), emtricitabine/rilpivirine/tenofovir (COMPLERA), abacavir/dolutegravir/lamivudine (TRIUMEQ), dasabuvir/ombitasvir/paritaprevir/ritonavir (VIEKIRA PAK), elbasvir/grazoprevir (ZEPATIER), lamivudine/zidovudine (COMBIVIR), cobicistat/elvitegravir/emtricitabine/tenofovir alafenamide (GENVOYA), cobicistat/darunavir (PREZCOBIX), emtricitabine/tenofovir, emtricitabine/lopinavir/ritonavir/tenofovir, emtricitabine/nelfinavir/tenofovir, lamivudine/raltegravir (DUTREBIS), atazanavir/cobicistat (EVOTAZ), interferon alfa-2b/ribavirin (REBETRON), ombitasvir/paritaprevir/ritonavir (TECHNIVIE), and combinations thereof.

Examples of antiviral interferons of use herein include, without limitation, peginterferon alfa-2a (PEGASYS), peginterferon alfa-2b (PEGINTRON), peginterferon alfa-2b (SYLATRON), and combinations thereof.

An exemplary chemokine receptor antagonist of use herein is maraviroc (SELZENTRY).

Exemplary integrase strand transfer inhibitors of use herein include, without limitation, raltegravir, dolutegravir, elvitegravir, and combinations thereof.

Exemplary non-nucleoside reverse transcriptase inhibitors (NNRTIs) of use herein include, without limitation, nevirapine, etravirine, efavirenz, rilpivirine, delavirdine, nevirapine and combinations thereof.

An exemplary non-structural protein 5A (NS5A) inhibitor of use herein is daclatasvir (DAKLINZA).

Exemplary nucleoside reverse transcriptase inhibitors (NRTIs) of use herein include, without limitation, entecavir, lamivudine, adefovir, didanosine, tenofovir, abacavir, lamivudine, zidovudine, stavudine, emtricitabine, zalcitabine, telbivudine, didanosine, and combinations thereof.

Exemplary protease inhibitors of use herein include, without limitation, boceprevir, simeprevir, telaprevir, lopinavir/ritonavir (KALETRA), fosamprenavir, darunavir, ritonavir, tipranavir, atazanavir, nelfinavir, amprenavir, indinavir, saquinavir, and combinations thereof.

Exemplary purine nucleoside of use herein include, without limitation, ribavirin, valacyclovir, famciclovir, acyclovir, ganciclovir, valganciclovir, cidofovir and combinations thereof.

Other exemplary antiviral agents of use herein include, without limitation, sofosbuvir, enfuvirtide, enfuvirtide, fomivirsen, and combinations thereof.

As used herein, the term “immunotherapeutic agent” refers to a molecule that can aid in the treatment of a disease by inducing, enhancing, or suppressing an immune response in a cell, tissue, organ or subject. Examples of immunotherapeutic agents contemplated for use in combination with at least one glutamine antagonist described herein include, but are not limited to, immune checkpoint molecules (e.g., antibodies to immune checkpoint proteins), interleukins (e.g., IL-2, IL-7, IL-12, IL-15), cytokines (e.g., interferons, G-CSF, imiquimod), chemokines (e.g., CCL3, CCL26, CXCL7), vaccines (e.g., peptide vaccines, dendritic cell (DC) vaccines, EGFRvIII vaccines, mesothilin vaccine, G-VAX, listeria vaccines), and adoptive T cell therapy including chimeric antigen receptor T cells (CAR T cells).

As used herein, “anti-inflammatory agent” refers to an agent that may be used to prevent or reduce an inflammatory response or inflammation in a cell, tissue, organ, or subject. Exemplary anti-inflammatory agents contemplated for use include, without limitation, steroidal anti-inflammatory agents, a nonsteroidal anti-inflammatory agent, or a combination thereof. In some embodiments, anti-inflammatory agents include clobetasol, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone, dexamethasone acetate, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus, prodrugs thereof, co-drugs thereof, and combinations thereof. The anti-inflammatory agent may also be a biological inhibitor of proinflammatory signaling molecules including antibodies to such biological inflammatory signaling molecules.

Exemplary neuroprotective agents include, without limitation, L-dopa, dopamine agonists (e.g., apomorphine, bromocriptine, pergolide, ropinirole, pramipexole, or cabergoline), adenosine A2a antagonists (Shah et al., Curr. Opin. Drug Discov. Devel. 13:466-80 (2010)); serotonin receptor agonists; continuous-release levodopa (Sinemet CR®, MSD, Israel); continuous duodenal levodopa administration (Duodopa®, Abbott, UK); catechol-O-methyltransferase (COMT) inhibitors (e.g., Stalevo®, Novartis Pharma, USA; entacapone (Comtan®, Novartis Pharma, USA)); tolcapone; coenzyme Q10, and/or MAO-B inhibitors (e.g., Selegiline or Rasagiline). Additional neuroprotective agents are described in, e.g., Hart et al., Mov. Disord. 24: 647-54 (2009).

In some contexts, an agent described herein can be administered with an antigen (e.g., to induce an immune response). In some embodiments, an adjuvant can be used in combination with the antigen.

An agent described herein can also be used in combination with an imaging agent. An agent (e.g., a glutamine antagonist) can be attached to imaging agents for imaging and diagnosis of various diseased organs, tissues or cell types. The agent can be labeled or conjugated a fluorophore or radiotracer for use as an imaging agent. Many appropriate imaging agents are known in the art, as are methods for their attachment to agents (e.g., attaching an imaging agent to a proteins or peptides using metal chelate complexes, radioisotopes, fluorescent markers, or enzymes whose presence can be detected using a colorimetric markers (such as, but not limited to, urease, alkaline phosphatase, (horseradish) hydrogen peroxidase and glucose oxidase)). An agent may also be dual labeled with a radioisotope in order to combine imaging through nuclear approaches and be made into a unique cyclic structure and optimized for binding affinity and pharmacokinetics. Such agents can be administered by any number of methods known to those of ordinary skill in the art including, but not limited to, oral administration, inhalation, subcutaneous (sub-q), intravenous (IV.), intraperitoneal (LP.), intramuscular (IM.), or intrathecal injection.

The presently disclosed subject matter contemplates the use of at least one glutamine antagonists, alone, or optionally together with one or more additional therapeutic agents described herein. Accordingly, in an aspect the presently disclosed subject matter involves the use of at least one glutamine antagonist for treating an acute kidney injury.

### II. Pharmaceutical Compositions Comprising Glutamine Antagonists

The presently disclosed subject matter also contemplates pharmaceutical compositions comprising one or more glutamine antagonists for the prevention or treatment of an acute kidney injury. In some embodiments, the presently disclosed methods comprise the use of the presently disclosed glutamine antagonists for the manufacture of a medicament for the treatment of an acute kidney injury.

Accordingly, in some embodiments, the presently disclosed subject matter provides a pharmaceutical composition comprising an effective amount of at least one glutamine antagonist that treats an acute kidney injury, and a pharmaceutically acceptable carrier, diluent, or excipient.

In some embodiments, the glutamine antagonist composition comprises one or more additional therapeutic agents described herein (e.g., antiviral agents, immunotherapeutic agents, anti-inflammatory agents, neuroprotective agents, neuroregenerative agents, neurotrophic factors, stem and progenitor cells used to replace and/or repair endogenous populations of abnormal, harmful, or unhealthy cells, and vaccines). Generally, the presently disclosed compositions (e.g., comprising at least one glutamine antagonist) can be administered to a subject for therapy by any suitable route of administration, including orally, nasally, transmucosally, ocularly, rectally, intravaginally, parenterally, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections, intracisternally, topically, as by powders, ointments or drops (including eyedrops), including buccally and sublingually, transdermally, through an inhalation spray, or other modes of delivery known in the art.

The phrases “systemic administration,” “administered systemically,” “peripheral administration” and “administered peripherally” as used herein mean the administration of compositions comprising at least one glutamine antagonist, such that it enters the patient's system and, thus, are subject to metabolism and other like processes, for example, subcutaneous administration.

The phrases “parenteral administration” and “administered parenterally” as used herein mean modes of administration other than enteral and topical administration, usually by injection, and includes, without limitation, intravenous, intramuscular, intraarterial, intrathecal, intracapsular, intraorbital, intraocular, intracardiac, intradermal, intraperitoneal, transtracheal, subcutaneous, subcuticular, intraarticular, subcapsular, subarachnoid, intraspinal and intrasternal injection and infusion.

The presently disclosed pharmaceutical compositions can be manufactured in a manner known in the art, e.g., by means of conventional mixing, dissolving, granulating, dragee-making, levitating, emulsifying, encapsulating, entrapping or lyophilizing processes.

In some embodiments, the presently disclosed pharmaceutical compositions can be administered by rechargeable or biodegradable devices. For example, a variety of slow-release polymeric devices have been developed and tested in vivo for the controlled delivery of drugs, including proteinaceous biopharmaceuticals. Suitable examples of sustained release preparations include semipermeable polymer matrices in the form of shaped articles, e.g., films or microcapsules. Sustained release matrices include polyesters, hydrogels, polylactides (U.S. Pat. No. 3,773,919; EP 58,481), copolymers of L-glutamic acid and gamma ethyl-L-glutamate (Sidman et al., Biopolymers 22:547, 1983), poly (2-hydroxyethyl-methacrylate) (Langer et al. (1981) J. Biomed Mater. Res. 15:167; Langer (1982), Chem. Tech. 12:98), ethylene vinyl acetate (Langer et al. (1981) J. Biomed Mater. Res. 15:167), or poly-D-(−)-3-hydroxybutyric acid (EP 133,988A). Sustained release compositions also include liposomally entrapped compositions comprising at least one glutamine antagonist which can be prepared by methods known in the art (Epstein et al. (1985) Proc. Natl. Acad Sci. U.S.A. 82:3688; Hwang et al. (1980) Proc. Natl. Acad Sci. U.S.A. 77:4030; U.S. Pat. Nos. 4,485,045 and 4,544,545; and EP 102,324A). Ordinarily, the liposomes are of the small (about 200-800 angstroms) unilamelar type in which the lipid content is greater than about 30 mol % cholesterol, the selected proportion being adjusted for the optimal therapy. Such materials can comprise an implant, for example, for sustained release of the presently disclosed compositions, which, in some embodiments, can be implanted at a particular, pre-determined target site.

In another embodiment, the presently disclosed pharmaceutical compositions may comprise PEGylated therapeutics (e.g., PEGylated antibodies). PEGylation is a well-established and validated approach for the modification of a range of antibodies, proteins, and peptides and involves the attachment of polyethylene glycol (PEG) at specific sites of the antibodies, proteins, and peptides (Chapman (2002) Adv. Drug Deliv. Rev. 54:531-545). Some effects of PEGylation include: (a) markedly improved circulating half-lives in vivo due to either evasion of renal clearance as a result of the polymer increasing the apparent size of the molecule to above the glomerular filtration limit, and/or through evasion of cellular clearance mechanisms; (b) improved pharmacokinetics; (c) improved solubility PEG has been found to be soluble in many different solvents, ranging from water to many organic solvents, such as toluene, methylene chloride, ethanol and acetone; (d) PEGylated antibody fragments can be concentrated to 200 mg/mL, and the ability to do so opens up formulation and dosing options, such as subcutaneous administration of a high protein dose; this is in contrast to many other therapeutic antibodies which are typically administered intravenously; (e) enhanced proteolytic resistance of the conjugated protein (Cunningham-Rundles et. al. (1992) J. Immunol. Meth. 152:177-190); (f) improved bioavailability via reduced losses at subcutaneous injection sites; (g) reduced toxicity has been observed; for agents where toxicity is related to peak plasma level, a flatter pharmacokinetic profile achieved by sub-cutaneous administration of PEGylated protein is advantageous; proteins that elicit an immune response which has toxicity consequences may also benefit as a result of PEGylation; and (h) improved thermal and mechanical stability of the PEGylated molecule.

Pharmaceutical compositions for parenteral administration include aqueous solutions of compositions comprising at least one glutamine antagonist. For injection, the presently disclosed pharmaceutical compositions can be formulated in aqueous solutions, for example, in some embodiments, in physiologically compatible buffers, such as Hank's solution, Ringer's solution, or physiologically buffered saline. Aqueous injection suspensions can contain substances that increase the viscosity of the suspension, such as sodium carboxymethyl cellulose, sorbitol, or dextran. Additionally, suspensions of compositions include fatty oils, such as sesame oil, or synthetic fatty acid esters, such as ethyl oleate or triglycerides, or liposomes. Optionally, the suspension also can contain suitable stabilizers or agents that increase the solubility of the compositions comprising at least one glutamine antagonist to allow for the preparation of highly concentrated solutions.

For nasal or transmucosal administration generally, penetrants appropriate to the particular barrier to be permeated are used in the formulation. Such penetrants are generally known in the art.

Additional ingredients can be added to compositions for topical administration, as long as such ingredients are pharmaceutically acceptable and not deleterious to the epithelial cells or their function. Further, such additional ingredients should not adversely affect the epithelial penetration efficiency of the composition, and should not cause deterioration in the stability of the composition. For example, fragrances, opacifiers, antioxidants, gelling agents, stabilizers, surfactants, emollients, coloring agents, preservatives, buffering agents, and the like can be present. The pH of the presently disclosed topical composition can be adjusted to a physiologically acceptable range of from about 6.0 to about 9.0 by adding buffering agents thereto such that the composition is physiologically compatible with a subject's skin.

Regardless of the route of administration selected, the presently disclosed compositions are formulated into pharmaceutically acceptable dosage forms, such as described herein or by other conventional methods known to those of skill in the art.

In general, the “effective amount”, “amount effective to treat” or “therapeutically effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the composition of the encapsulating matrix, the target tissue, and the like. Generally, the “effective amount” or “amount effective” to treat an acute kidney injury is less than the effective amount needed to treat cancer. It should be appreciated that the amount of at least one glutamine antagonist, or prodrug or analog thereof, effective to treat an acute kidney injury comprises the maximal non-toxic dose that sufficient for improving a particular acute kidney injury in a subject. In some embodiments, the effective amount of at least one glutamine antagonist, or prodrug or analog thereof, is less than 0.1 mg/kg/day.

The term “combination” is used in its broadest sense and means that a subject is administered at least two agents. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the presently disclosed compositions can be administered alone or in combination with adjuvants that enhance stability of the agents, facilitate administration of pharmaceutical compositions containing them in certain embodiments, provide increased dissolution or dispersion, increase activity, provide adjuvant therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of at least one glutamine antagonist can be varied so long as the beneficial effects of the combination of these agents are achieved. Accordingly, the phrase “in combination with” refers to the administration of at least one glutamine antagonist, and optionally additional agents either simultaneously, sequentially, or a combination thereof. Therefore, a subject administered a combination of at least one glutamine antagonist, and optionally additional agents can receive at least one glutamine antagonist, and optionally additional agents at the same time (i.e., simultaneously) or at different times (i.e., sequentially, in either order, on the same day or on different days), so long as the effect of the combination of all agents is achieved in the subject.

When administered sequentially, the agents can be administered within 1, 5, 10, 30, 60, 120, 180, 240 minutes or longer of one another. In other embodiments, agents administered sequentially, can be administered within 1, 2, 3, 4, 5, 10, 15, 20 or more days of one another. Where the agents are administered simultaneously, they can be administered to the subject as separate pharmaceutical compositions, each comprising at least one glutamine antagonist, and optionally additional agents, or they can be administered to a subject as a single pharmaceutical composition comprising all agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of an agent and at least one additional therapeutic agent is greater than the sum of the biological activities of the respective agents when administered individually.

Synergy can be expressed in terms of a “Synergy Index (SI),” which generally can be determined by the method described by F. C. Kull et al. Applied Microbiology 9, 538 (1961), from the ratio determined by:

QₐQ_{A}+Q_{b}Q_{B}=Synergy Index(SI)

wherein:
Q_{A} is the concentration of a component A, acting alone, which produced an end point in relation to component A;
Qa is the concentration of component A, in a mixture, which produced an end point;
QB is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

In another aspect, the presently disclosed subject matter provides a pharmaceutical composition including at least one glutamine antagonist, and optionally additional agents, alone or in combination with one or more additional therapeutic agents in admixture with a pharmaceutically acceptable excipient.

More particularly, the presently disclosed subject matter provides a pharmaceutical composition comprising at least one glutamine antagonist, and optionally additional agents, and a pharmaceutically acceptable carrier. In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams and Wilkins (2000).

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

### III. General Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs. While the following terms in relation to compounds of Formula (I) are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group on a molecule, provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted at one or more positions).

Where substituent groups or linking groups are specified by their conventional chemical formulae, written from left to right, they equally encompass the chemically identical substituents that would result from writing the structure from right to left, e.g., —CH₂O— is equivalent to —OCH₂—; —C(═O)O— is equivalent to —OC(═O)—; —OC(═O)NR— is equivalent to —NRC(═O)O—, and the like.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds. Unless otherwise explicitly defined, a “substituent group,” as used herein, includes a functional group selected from one or more of the following moieties, which are defined herein:

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, isopropyl, cyclopropyl, allyl, vinyl, n-butyl, tert-butyl, ethynyl, cyclohexyl, and the like.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁-C₁₀ means one to ten carbons, including 1, 2, 3, 4, 5, 6, 7, 8, 9, and 10 carbons). In particular embodiments, the term “alkyl” refers to C₁₋₂₀ inclusive, including 1, 2, 3, 4, 5, 6, 7, 8, 9, 10, 11, 12, 13, 14, 15, 16, 17, 18, 19, and 20 carbons, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof.

“Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “heteroalkyl,” by itself or in combination with another term, means, unless otherwise stated, a stable straight or branched chain, or cyclic hydrocarbon group, or combinations thereof, consisting of at least one carbon atoms and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂₅—S(O)—CH₃, —CH₂—CH₂—S(O)₂—CH₃, —CH═CHO—CH₃, —Si(CH₃)₃, —CH₂—CH═N—OCH₃, —CH═CH—N(CH₃)— CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)NR′, —NR′R″, —OR′, —SR, —S(O)R, and/or —S(O₂)R′. Where “heteroalkyl” is recited, followed by recitations of specific heteroalkyl groups, such as —NR′R or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, unsubstituted alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

The term “cycloalkylalkyl,” as used herein, refers to a cycloalkyl group as defined hereinabove, which is attached to the parent molecular moiety through an alkyl group, also as defined above. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur (S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.”

More particularly, the term “alkenyl” as used herein refers to a monovalent group derived from a C₁₋₂₀ inclusive straight or branched hydrocarbon moiety having at least one carbon-carbon double bond by the removal of a single hydrogen molecule. Alkenyl groups include, for example, ethenyl (i.e., vinyl), propenyl, butenyl, 1-methyl-2-buten-1-yl, pentenyl, hexenyl, octenyl, allenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₁₋₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, and heptynyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂—and —CH₂—S—CH₂—CH₂—NH—CH₂—. For heteroalkylene groups, heteroatoms also can occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′—and —R′OC(O)—.

The term “aryl” means, unless otherwise stated, an aromatic hydrocarbon substituent that can be a single ring or multiple rings (such as from 1 to 3 rings), which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxy, arylthioxy, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover only aryls substituted with one or more halogens.

Where a heteroalkyl, heterocycloalkyl, or heteroaryl includes a specific number of members (e.g. “3 to 7 membered”), the term “member” refers to a carbon or heteroatom.

Further, a structure represented generally by the formula:

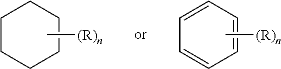

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

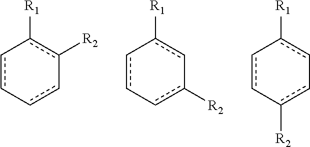

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

The symbol ( [symbol] ) denotes the point of attachment of a moiety to the remainder of the molecule.

When a named atom of an aromatic ring or a heterocyclic aromatic ring is defined as being “absent,” the named atom is replaced by a direct bond.

Each of above terms (e.g., “alkyl,” “heteroalkyl,” “cycloalkyl, and “heterocycloalkyl”, “aryl,” “heteroaryl,” “phosphonate,” and “sulfonate” as well as their divalent derivatives) are meant to include both substituted and unsubstituted forms of the indicated group. Optional substituents for each type of group are provided below.

Substituents for alkyl, heteroalkyl, cycloalkyl, heterocycloalkyl monovalent and divalent derivative groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such groups. R′, R″, R′″ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. As used herein, an “alkoxy” group is an alkyl attached to the remainder of the molecule through a divalent oxygen. When a compound of the disclosure includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl groups above, exemplary substituents for aryl and heteroaryl groups (as well as their divalent derivatives) are varied and are selected from, for example: halogen, —OR′, —NR′R″, —SR′, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)═NR″″, —NR—C(NR′R″)═NR′—S(O)R′, —S(O)₂R′, —S(O)₂NR′R″, —NRSO₂R′, —CN and —NO₂, —R′, —N₃, —CH(Ph)₂, fluoro(C₁-C₄)alkoxo, and fluoro(C₁-C₄)alkyl, in a number ranging from zero to the total number of open valences on aromatic ring system; and where R′, R″, R′″ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ—B—, wherein A and B are independently —CRR′—, —O—, —NR—, —O—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as a 2-(furan-2-yl)acetyl)- and a 2-phenylacetyl group. Specific examples of acyl groups include acetyl and benzoyl. Acyl groups also are intended to include amides, —RC(═O)NR′, esters, —RC(═O)OR′, ketones, —RC(═O)R′, and aldehydes, —RC(═O)H.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl, i.e., C₆H₅—CH₂—O—. An aralkyloxyl group can optionally be substituted.

“Alkoxycarbonyl” refers to an alkyl-O—C(═O)— group. Exemplary alkoxycarbonyl groups include methoxycarbonyl, ethoxycarbonyl, butyloxycarbonyl, and tert-butyloxycarbonyl.

“Aryloxycarbonyl” refers to an aryl-O—C(═O)— group. Exemplary aryloxycarbonyl groups include phenoxy- and naphthoxy-carbonyl.

“Aralkoxycarbonyl” refers to an aralkyl-O—C(═O)— group. An exemplary aralkoxycarbonyl group is benzyloxycarbonyl.

“Carbamoyl” refers to an amide group of the formula —C(═O)NH₂. “Alkylcarbamoyl” refers to a R′RN—C(═O)— group wherein one of R and R′ is hydrogen and the other of R and R′ is alkyl and/or substituted alkyl as previously described. “Dialkylcarbamoyl” refers to a R′RN—C(═O)— group wherein each of R and R′ is independently alkyl and/or substituted alkyl as previously described.

The term carbonyldioxyl, as used herein, refers to a carbonate group of the formula —O—C(═O)—OR.

“Acyloxyl” refers to an acyl-O— group wherein acyl is as previously described.

The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups, respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R′″ are each independently selected from the group consisting of alkyl groups. Additionally, R′, R″, and/or R′″ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, isopropylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to the —C(═O)— group, and can include an aldehyde group represented by the general formula R—C(═O)H.

The term “carboxyl” refers to the —COOH group. Such groups also are referred to herein as a “carboxylic acid” moiety.

The terms “halo,” “halide,” or “halogen” as used herein refer to fluoro, chloro, bromo, and iodo groups. Additionally, terms, such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

More particularly, the term “sulfide” refers to compound having a group of the formula —SR.

The term “sulfone” refers to compound having a sulfonyl group —S(O₂)R.

The term “sulfoxide” refers to a compound having a sulfinyl group —S(O)R The term ureido refers to a urea group of the formula —NH—CO—NH₂.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as D- or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)-, or D- and L-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds which differ only in the presence of one or more isotopically enriched atoms. For example, compounds having the present structures with the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are encompassed within the scope of the present disclosure.

The compounds of the present disclosure may exist as salts. The present disclosure includes such salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures, succinates, benzoates and salts with amino acids, such as glutamic acid. These salts may be prepared by methods known to those skilled in art. Also included are base addition salts, such as sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent or by ion exchange. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids, such as arginate and the like, and salts of organic acids like glucuronic or galactunoric acids and the like. Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments 0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration and are not to be construed as limiting in any manner to make compounds of the disclosure by other methods.

### Example 1

### T Cell Metabolic Reprogramming and Effect of Glutamine Blockade in Ischemic Acute Kidney Injury

### 1.1 Overview

T cells play important roles in the pathogenesis of ischemic acute kidney injury (AKI). Metabolic programming of T cells directly regulates T cell function, which is a rapidly emerging field, but has not been studied in detail during AKI. Among metabolic pathways, the glutaminolysis pathway on T cells has been suggested as an important metabolic checkpoint, because activated T cells utilize glutamine metabolism to fuel high proliferative rates. See, for example, Rabb et al., 2000, and Pearce, et al., 2013.

### 1.2 Methods and Results

Without being bound to any one particular theory, it is thought that T cells undergo metabolic reprogramming during ischemic acute kidney injury and modulation of glutamine pathway alters T cell activation and function by which affects renal outcomes.

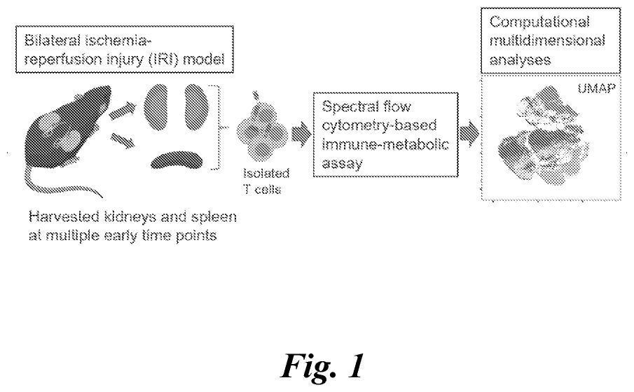

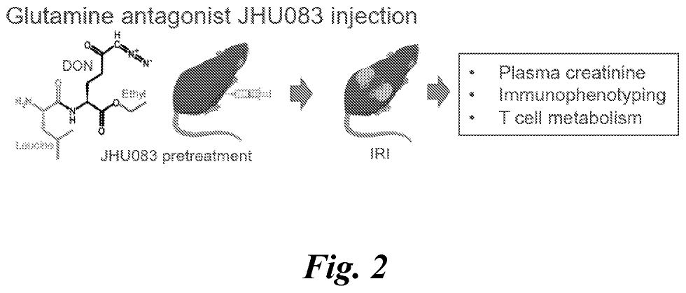

To test this hypothesis, referring now to FIG. 1 and FIG. 2, acute kidney injury was induced with 30-min ischemia followed by reperfusion in C57 black 6 mice. Kidneys and spleens were harvested at multiple time points. Subsequently, spectral flow cytometry based immune-metabolic assay was utilized. The data were evaluated by UMAP multidimensional analyses. Further, to study the effect of glutamine blockade, the glutamine antagonist referred to herein as JHU083 was injected intraperitoneally.

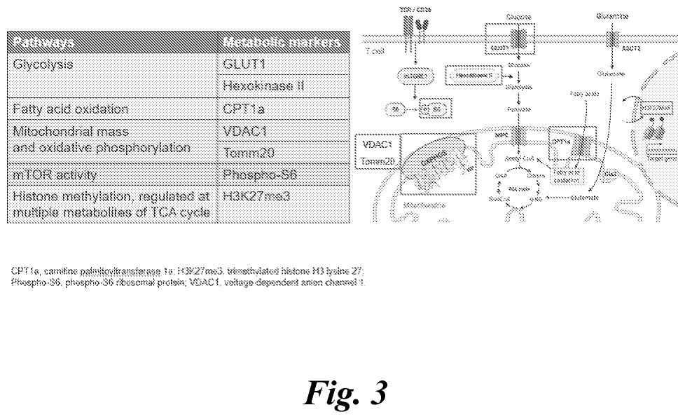

Referring now to FIG. 3, to assess T cell metabolism, the following metabolic markers were included in the assay. The glucose transporter, GLUT1 and the rate limiting enzyme of glycolysis, hexokinase II were evaluated. The fatty acid oxidation was measured by expression of CPT1a. Mitochondrial membrane proteins, voltage-dependent anion channel 1 (VDAC1) and Tomm20 were used as markers for mitochondrial oxidation. mTOR activity was measured through phospho-S6. H3K27me3 also was included as an epigenetic marker, which is regulated at multiple metabolites.

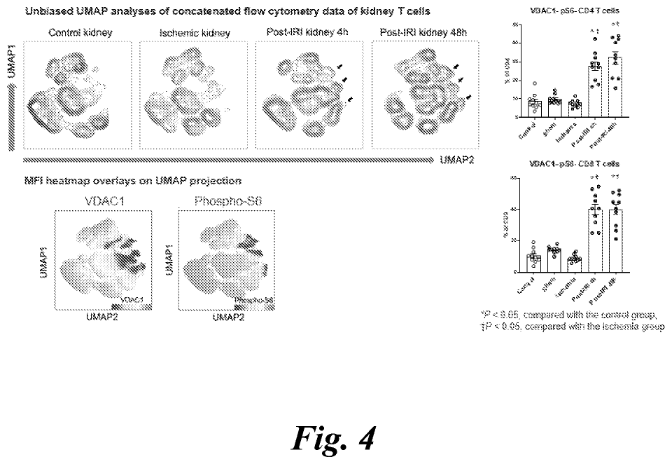

Referring now to FIG. 4, the results of unbiased clustering of T cells from control kidneys, ischemic kidneys, and post-IRI kidneys are shown. The arrows indicate distinct T cell populations in the post-IRI kidneys. In the bottom row, heatmap overlays indicate those populations have lower VDAC1 and phospho-S6 expression, so these two metabolic markers drove the segregation of post-IRI clusters. As such, a distinct T cell subset with lower expression of VDAC1 and phospho-S6 proteins in postischemic kidneys was identified.

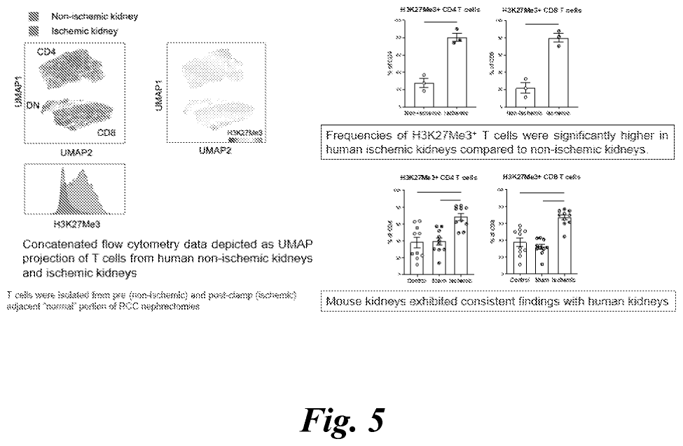

Referring now to FIG. 5, the effect of ischemia on human kidney T cell metabolism was assessed. T cells for this analysis were obtained from normal portions of RCC nephrectomy kidneys. H3K27Me3 expression, which is regulated by TCA cycle, segregated T cells from non-ischemic and ischemic kidneys. It was found that T cells from ischemic kidneys have higher H3K27Me3 expression compared to non-ischemic kidneys. Mouse data also showed consistent findings between non-ischemic and ischemic kidneys.

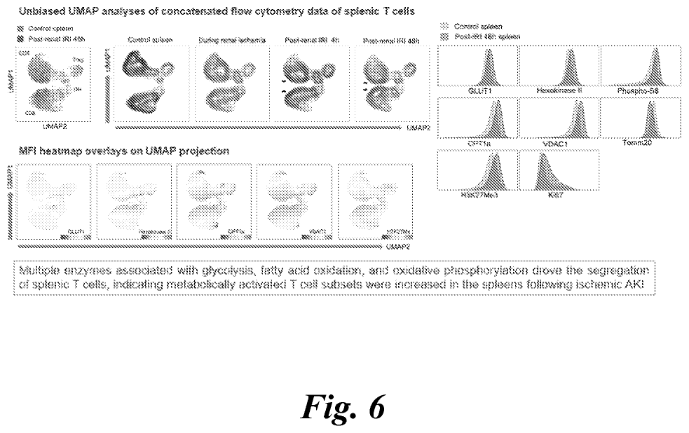

Referring now to FIG. 6, in splenic T cells, their metabolic profiles were changed dynamically following ischemic acute kidney injury. The enzymes associated with glycolysis, fatty acid oxidation, and mitochondrial oxidation were upregulated significantly after reperfusion. These results show that extrarenal T cells are metabolically activated after ischemic acute kidney injury.

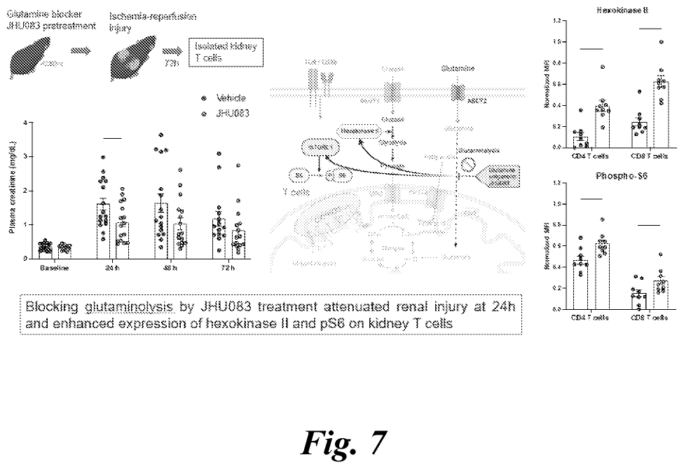

Referring now to FIG. 7, because activated T cells are highly dependent on glutaminolysis, the effect of glutamine blockade on T cell metabolism during AKI was tested. The mice were treated with glutamine antagonist, JHU083, before inducing AKI. JHU083 blocks a broad range of glutamine-requiring enzymes including the glutaminase. We followed-up until 72 hrs after the surgery and isolated T cells from postischemic kidneys. Referring now to the left hand side of FIG. 7, it was found that glutamine blockade attenuated renal injury at 24 hr. As shown on the right hand side of FIG. 7, it enhanced expression of enzymes involved in glycolysis and mTOR activity on kidney T cells.

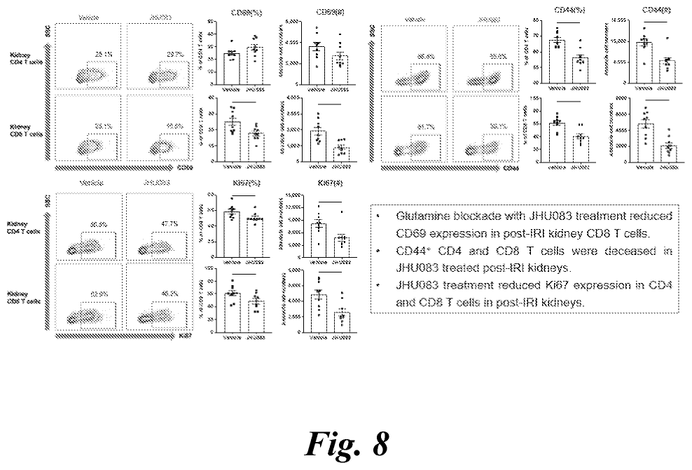

Referring now to FIG. 8, the immuno-phenotypic effect of JHU083 on kidney CD4⁺ and CD8⁺ T cells also was studied. The T cells from mice treated with the glutamine antagonist showed reduced expression of CD69, CD44, and Ki67, compared to the vehicle control group. The glutamine blockade is likely to suppress kidney T cell activation and proliferation in vivo after ischemic AKI.

### 1.3 Summary

First, metabolic enzymes on kidney T cells involved in mitochondrial oxidation and mTOR activities were changed following ischemic AKI. Second, overall metabolic pathways in splenic T cells were upregulated after ischemic AKI. Third, glutamine blockade affected functional renal outcome and reduced T cell activation and proliferation in post-AKI kidneys.

In summary, T cells undergo distinct metabolic reprogramming during ischemic AKI. Reconstitution of T cell metabolism by targeting the glutamine pathway could be a promising therapeutic approach for acute kidney injury.

### Example 2

### T Cell Metabolic Reprogramming in Acute Kidney Injury and Protection by Glutamine Blockade

### 2.1 Overview

T cells play an important role in acute kidney injury (AKI). Metabolic programming of T cells regulates their function, is a rapidly emerging field, and is unknown in AKI. We induced ischemic AKI in C57B6 mice and collected kidneys and spleens at multiple time points. T cells were isolated and analyzed by a spectral flow cytometry-based immune-metabolic assay. Unbiased multidimensional machine learning analyses identified a distinct T cell subset with reduced mitochondrial VDAC1 and mTOR expression in post-AKI kidneys. H3K27Me3 expression separated ischemic kidney T cells from those of nonischemic kidneys. Splenic T cells from post-AKI mice had higher expression of GLUT1, hexokinase II, and CPT1a. Human nonischemic and ischemic kidney tissue was obtained from nephrectomy cases, with similar findings to mouse kidneys. Given a convergent role for glutamine in many T cell metabolic pathways and the availability of a safe glutamine antagonist JHU083, effects on AKI were evaluated. JHU083 treatment attenuated renal injury and reduced T cell activation and proliferation in both ischemic and nephrotoxic AKI model. In vitro hypoxia demonstrated upregulation of glycolysis-related enzymes. T cells undergo metabolic reprogramming during AKI, and reconstitution of metabolism by targeting T cell glutamine pathway could be a promising novel therapeutic approach.

### 2.2 Background

Acute kidney injury (AKI) is an important clinical problem affecting both native kidneys and renal allografts. There is no specific treatment for AKI except for supportive measures including fluid therapy or dialysis. Kellum et al., 2021. There are many cellular and molecular mechanisms involved in AKI pathogenesis including inflammation, epigenetics, cell death pathways, epithelial cell metabolic abnormalities, and others. Aufhauser et al., 2016; Bonventre and Yang, 2011; Jang and Rabb, 2015; Wei et al., 2018; Yang et al., 2010; Ying et al., 2019. T cells are established to play a modulatory role in AKI and repair. Burne et al., 2001; Rabb et al., 2000.

Metabolic reprogramming has emerged as a central mechanism of T cell activation and differentiation. Pearce et al., 2013. Metabolic pathways, such as glutaminolysis, glycolysis, fatty acid oxidation, and oxidative phosphorylation (OXPHOS) and their metabolites, were traditionally considered downstream consequences of cellular function. Buck et al., 2017. There is an increasing recognition, however, that these metabolic pathways play important and coordinated roles as regulators promoting differentiation and activation of T cells. Buck et al., 2017; Baumann et al., 2020; Buck et al., 2016.

During the past decade, there have been substantial advances in understanding T cell metabolic programming through various disease models, Patel et al., 2019; Teng et al., 2019; Sears et al., 2021; Zou et al., 2020, but this has not been studied in the context of AKI. T cells are predicted to undergo metabolic reprogramming during AKI since it is known that their numbers increase and are activated early during AKI. Ascon et al., 2006; Lai et al., 2007. Furthermore, considering that ischemic AKI results from exposure to hypoxia followed by reoxygenation, kidney T cells may utilize alternative energy sources during the ischemia and after reperfusion to maintain their effector function.

Reconstitution of metabolic pathways using specific inhibitory molecules can have immunomodulatory effects. Patel et al., 2019; Oh et al., 2020; Bettencourt and Powell, 2017. Given the high metabolic demands of effector T cells, blocking metabolic pathways can affect T cells selectively, not altering many normal cellular homeostatic functions which have more metabolically flexible mechanisms. Patel et al., 2019; Lee et al., 2015. Among metabolic pathways, the glutaminolysis pathway is among the important metabolic checkpoints in T cells, since activated T cells utilize aerobic glutamine metabolism to fuel high proliferative rates. Leone et al. 2019. Given the important role of T cells as an early responder in AKI, Rabb et al., 2000, modulation of the glutamine pathway using a specific inhibitory agent could modify T cell function and influence renal outcomes after AKI.

Without wishing to be bound to anyone particular theory, it is thought that T cells undergo metabolic reprogramming during experimental AKI. To study the metabolic landscape of T cells, we used spectral flow cytometry-based immune-metabolic assay with combining unsupervised computational analyses to identify metabolically dysregulated T cell subset in post-ischemic mouse kidneys. Thompson et al., 2021. We then studied human samples to determine clinical relevance. After finding pronounced and broad changes in T cell metabolism during AKI, we chose a select metabolic pathway, glutamine utilization, where many of the abnormalities converged and an interventional agent was available in which efficacy and safety has been shown in cancer models. Glutamine blockade was performed with the glutamine antagonist JHU083 in both ischemic and nephrotoxic AKI models in mice. We found that the JHU083 treatment changed kidney T cells to naïve phenotype, and improved functional and structural renal injury. To further identify mechanisms of JHU083 action, we studied the effects of JHU083 on in vitro hypoxia reoxygenation of T cells. Our findings demonstrate key T cell metabolic changes in murine and human AKI, and that reconstitution of T cells metabolism in AKI could be a novel therapeutic strategy for AKI.

### 2.3 Results

### 2.3.1 Immune-Metabolic Assay and Metabolic Signature of Activated T Cells

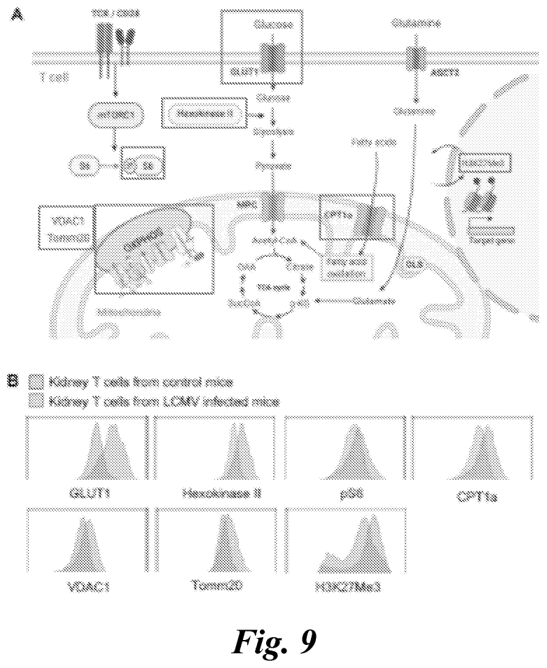

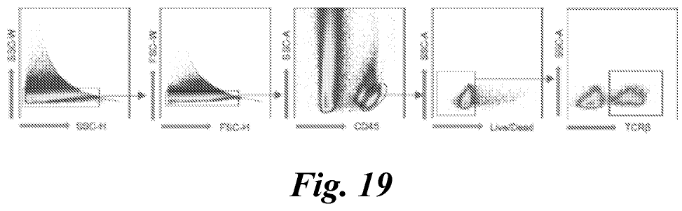

A spectral flow cytometry-based immune-metabolic assay was used to study T cell metabolic programs (FIG. 9A). To assess the glycolytic machinery, glucose transporter GLUT1 and the rate-limiting enzyme of glycolysis, hexokinase II (HKII) were evaluated. Fatty acid oxidation was measured by a rate limiting enzyme, carnitine palmitoyltransferase 1a (CPT1a) expression. mTOR signaling activity was measured by S6 ribosomal protein phosphorylation (pS6). Mitochondrial OXPHOS was assessed using mitochondrial membrane proteins, voltage-dependent anion channel 1 (VDAC1) and Tomm20. Since changes in cellular metabolism lead to epigenetic modulation of T cells through metabolites, we measured H3K27Me3, which is controlled by metabolites of the TCA cycle, as a readout for histone methylation. Pearce and Shen, 2006. The gating strategies for kidney T cells are provided in FIG. 19.

To establish a system to measure the metabolic signature of activated T cells with a positive disease control, T cells from lymphocytic choriomeningitis virus (LCMV) infected mouse kidneys were analyzed on day 7 after virus inoculation and compared with those from normal control mice. LCMV is known to directly infect kidneys and activate T cells in noncytopathic manner. Hotchin, 1971. Enzymes involved in glycolysis, OXPHOS, fatty acid oxidation, and mTOR activity were globally upregulated, whereas the repressive histone methylation marker H3K27me3 was down regulated in kidney T cells from LCMV infected mice (FIG. 9B).

2.3.2 Post-Ischemic Kidneys Revealed a Distinct T Cell Subset with Impaired mTOR and OXPHOS Activity

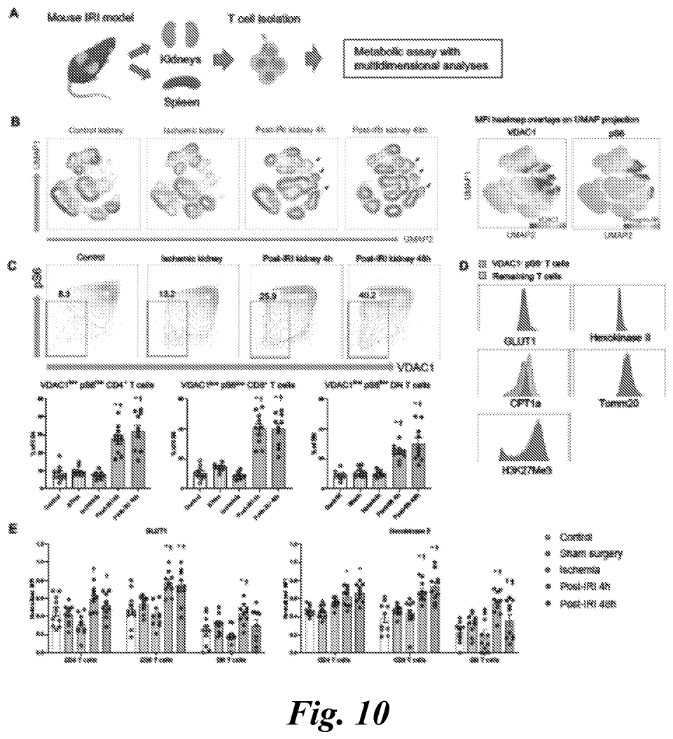

To elucidate T cell metabolic reprogramming in ischemic AKI, we induced bilateral ischemia-reperfusion injury (IRI) for 30 min and procured kidneys at multiple early post-reperfusion time points, including during ischemia, post-IRI 4 h, and post-IRI 48 h. Kidney T cells were isolated with an established technique, Ascon et al., 2006, and analyzed with high-dimensional unbiased analyses using Uniform Manifold Approximation and Projection (UMAP) algorithm (FIG. 10A).

Unsupervised multidimensional analyses revealed a distinct T cell population in post-ischemic kidneys, with low expression of mTOR activity, assessed by pS6, and VDAC1 (FIG. 10B). Based on this observation, we subsequently gated VDAC1^{low} and pS6^{low} population, and the percentages of this population were significantly increased in post-IRI kidneys compared to those from control kidneys and sham surgery kidneys (FIG. 10C). The VDAC1^{low} and pS6^{low} T cells were not limited to specific immunophenotypic populations, involving both effector-memory and naïve subsets of CD4⁺ and CD8⁺ T cells, as well as double-negative (DN) T cells. These distinct T cell subsets expressed a lower level of CPT1a compared to the remaining T cells, but they maintained comparable levels of glycolytic enzyme expression (FIG. 10D). This finding may indicate that T cells reduce OXPHOS and fatty oxidation under ischemic AKI and utilize glycolysis selectively. Within VDAC1^{low} pS6^{low} T cell subsets, GLUT1 and HKII expression were upregulated after reperfusion compared to those of control kidneys (FIG. 10E).

### 2.3.3 Metabolic Reprogramming of Splenic T Cells in Ischemic AKI

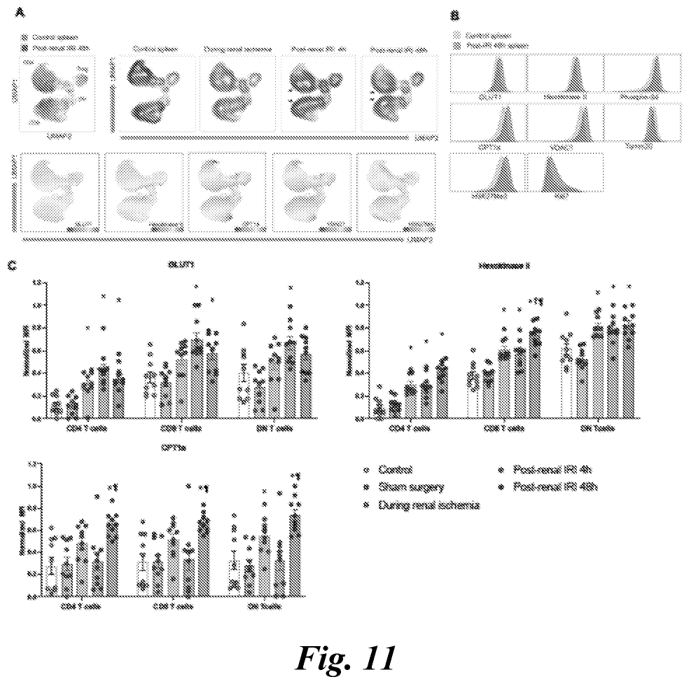

Given that AKI has a systemic immunologic effect, Lee et al., 2018, splenic T cells isolated from mice that underwent renal IRI also were studied at multiple time points. It also is easier to isolate and study the large number of T cells in spleen compared to kidney. Unbiased multidimensional analyses showed higher expression of GLUT1, HKII, CPT1a, VDAC1, and H3K27Me3 in post-IRI mice compared to the control mice (FIG. 11A). Splenic T cells from post-IRI mice exhibited phenotypes indicative of higher metabolic activity compared to those from the control and sham surgery mice with higher expression of GLUT1, HKII, and CPT1a, indicating an upregulation of glycolysis and fatty acid oxidation machinery (FIG. 11B and FIG. 11C). Since T cells in spleens are not directly exposed to metabolic stress from ischemia, unlike kidney T cells, these findings demonstrate the remote immunologic effect of T cell metabolism during AKI.

### 2.3.4 In Vitro Hypoxia LED to Upregulation of Glycolysis in Kidney T Cells

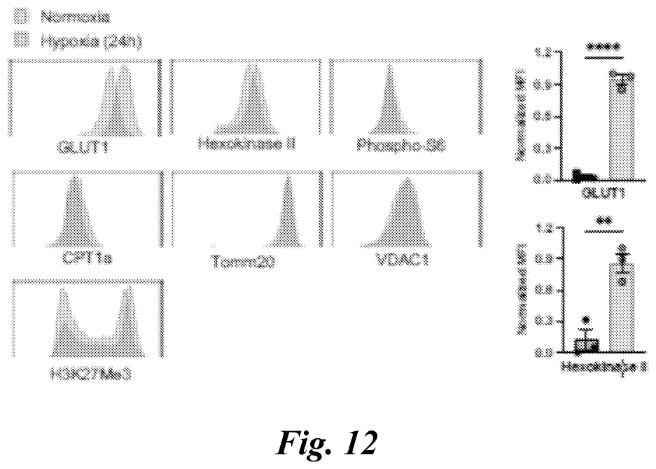

To test the effect of in vitro hypoxia followed by reoxygenation on activated T cell metabolism, FACS sorted kidney T cells (CD45⁺, TCR αβ⁺) were cultured with CD3/CD28 stimulation and incubated in a hypoxic chamber for 24 h followed by reoxygenation under normoxia for 24 h. T cells exposed to hypoxia showed upregulation of enzymes involved in glycolysis machinery including GLUT1 and HKII, compared to T cells under normoxic condition (FIG. 12). The in vitro anaerobic environment appeared to induce enhanced glycolysis in activated kidney T cells. Unlike in vivo data, however, the other enzymes were not downregulated following hypoxia exposure.

### 2.3.5 H3K27Me3 Expression Distinguishes T Cells in Non-Ischemic and Ischemic Human Kidneys

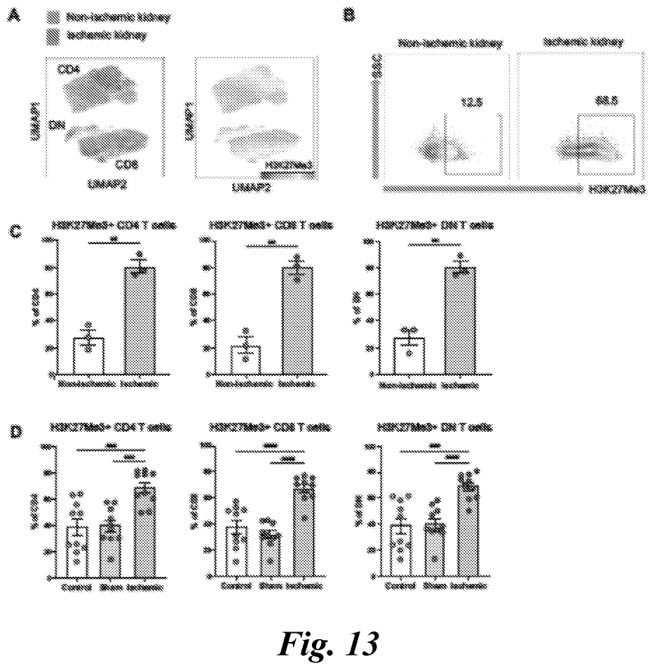

To evaluate human kidney T cell metabolic reprogramming in ischemic AKI, human non-ischemic (before clamping of renal artery) and ischemic kidney tissues (after clamping of renal artery) were procured separately from “normal” portions of renal cell carcinoma nephrectomy kidneys. In unbiased multidimensional analyses, upregulated H3K27Me3 expression drove the separation of ischemic kidney T cells from non-ischemic kidneys (FIG. 13A). The proportion of H3K27Me3⁺ T cell subsets was higher in ischemic kidneys compared to the non-ischemic kidneys (FIG. 13B and FIG. 13C). This increased methylation corresponds to decreased activity of histone demethylase enzyme. Patel et al., 2019. Since histone demethylase enzyme requires oxygen for their TCA cycle-dependent activation, Patel et al., 2019, hypoxic microenvironment during ischemia appears to downregulate its activity. This early epigenetic change may represent the initiation process needed to drive subsequent metabolic rewiring of T cells in post-AKI kidneys. No other metabolic enzymes were significantly changed in ischemic kidneys, perhaps due to the early time point assessment after ischemia and the lack of reperfusion as with mice. Data from mouse IRI model exhibited consistent findings, showing higher H3K27Me3 in T cells from ischemic kidneys than those from control mice or sham surgery mice (FIG. 13D).

### 2.3.6 Glutamine Blockade Exhibited Protective Effect and Reduced Leukocyte Infiltration in Ischemic AKI

Since glutamine utilization is essential for metabolic regulation of effector T cell activation and function, and a therapeutic agent is available that was safe in cancer models, we induced T cell metabolic reprogramming through glutamine antagonism and studied its effect on two different murine AKI models. Since in vivo use of conventional glutamine antagonists has been hindered by their dose-limiting toxicity, Lemberg et al., 2018, we utilized a recently developed prodrug of 6-Diazo-5-oxo-L-norleucine (DON), termed JHU083. Rais et al., 2016. JHU083 inhibits a broad range of enzymes involved in glutamine metabolism, including rate-limiting enzyme glutaminase, with improved safety profiles and bioavailability. Lemberg et al., 2018.

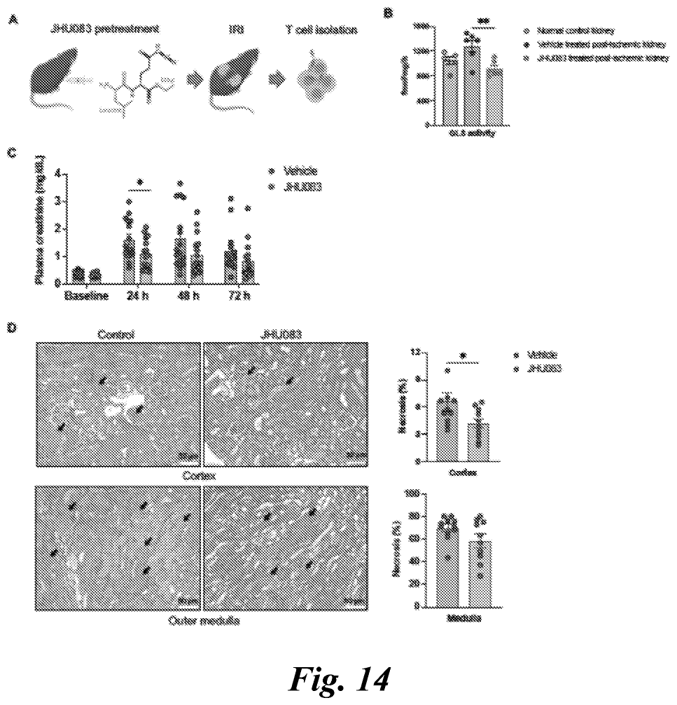

Mice were treated with 1.83 mg/kg of JHU083 or vehicle every other day via intraperitoneal injection and underwent bilateral IRI surgery on day 7 after the initial injection. This dosage regimen has been proven to be tolerable without significant toxicity even with a longer duration of treatment by a previous study. Hollinger et al., 2020. Mice were followed up until 72 h after IRI, and T cells isolated from post-ischemic kidneys were studied (FIG. 14A).

To confirm that JHU083 suppressed glutamine-related enzymes in kidneys, glutaminase activities were measured in post-ischemic kidney tissues at 24 h after IRI. Glutaminase activity showed a trend toward higher levels in vehicle-treated post-ischemic kidneys (23% increase, P=0.085) compared to the normal kidneys. Treatment with JHU083 significantly reduced glutaminase activities (29% decrease, P=0.008) (FIG. 14B).

The JHU083 treated mice showed significantly attenuated functional (plasma creatinine at 24 h, vehicle control vs JHU083, 1.61±0.18 vs 1.07±0.14 mg/dL, P=0.026) and structural kidney injury (cortical necrotic tubules, 6.6±0.9 vs 4.2±0.5%, P=0.039; medullary necrotic tubules, 69.7±3.1 vs 58.3±5.7%, P=0.093) (FIG. 14C and FIG. 14D).

### 2.3.7 Glutamine Blockade Reduced T Cell Activation and Proliferation in Post-Ischemic Kidneys

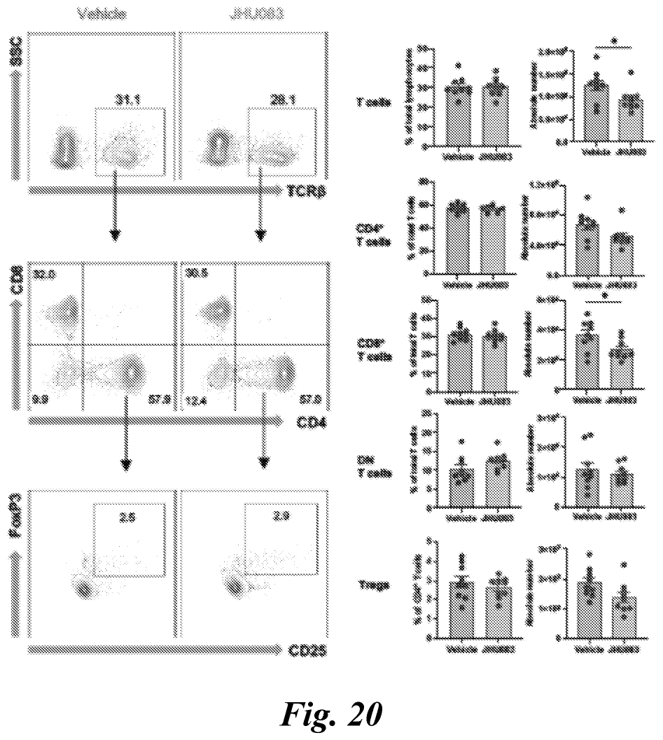

We analyzed kidney mononuclear cells (KMNCs) in post-ischemic kidneys to study the effect of JHU083 on T cells. The JHU083 treatment reduced the number of total T cells in post-ischemic kidneys. The proportions of CD4⁺, CD8⁺, double-negative (DN) T cells among total T cells (CD45⁺, TCR αβ⁺) and regulatory T cells (Tregs) among CD4⁺ T cells were comparable between groups (FIG. 20).

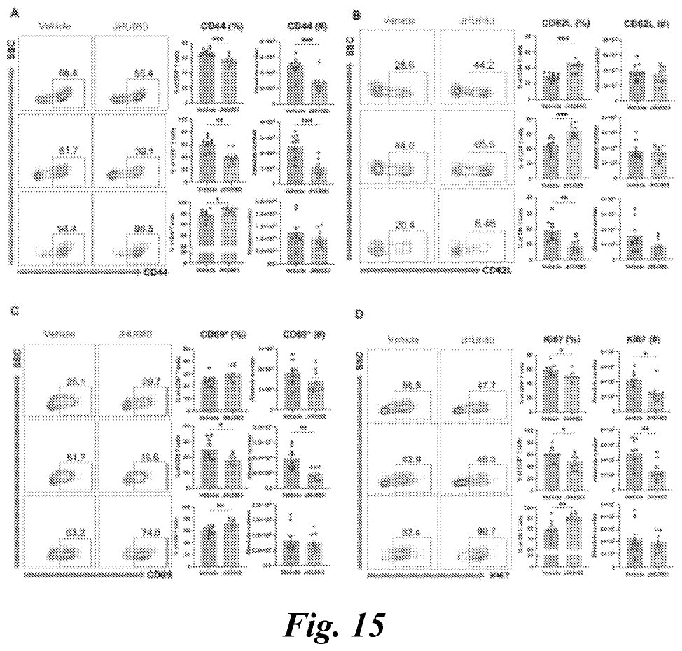

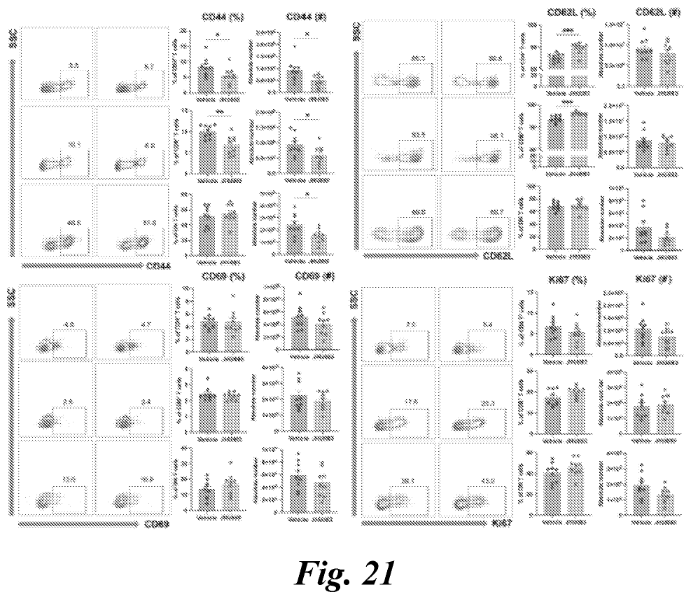

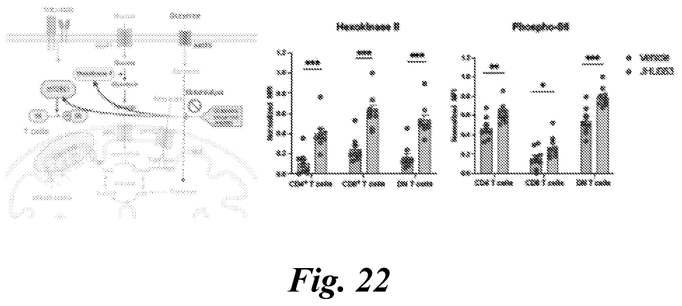

Further immunophenotypic characterization of T cells in postischemic kidneys demonstrated that the JHU083 treatment reduced effector and activated phenotypes of CD4⁺ (CD44⁺, 67.5±1.7 vs 56.3±1.9%, P<0.001; CD62L⁺, 29.0±1.6 vs 42.8±2.2, P<0.001) and CD8⁺ T cells (CD44⁺, 61.4±10.7 vs 40.9±11.2%, P=0.001; CD69⁺, 24.9±2.3 vs 17.8±1.4%, P=0.019; CD62L⁺, 44.9±3.2 vs 63.6±2.8, P<0.001) (FIG. 15). T cell proliferation assessed with Ki67 expression was reduced in CD4⁺ and CD8⁺ T cells from JHU083 treated post-ischemic kidneys (Ki67⁺, CD4⁺ T cells 58.7±2.9 vs 50.6±2.1%, P=0.035; CD8⁺ T cells, 61.2±4.2% vs 48.4±4.1%, P=0.043) (FIG. 15). JHU083 treatment did not reduce activation and proliferation of kidney DN T cells, the unconventional subset of kidney αβ T cells, rather modestly enhanced activation and proliferation (CD44⁺, 94.4±2.4 vs 96.4±1.2%, P=0.038; CD69⁺, 60.6±2.5 vs 72.1±2.5%, P=0.005; Ki67⁺, 78.7±3.0% vs 91.2±1.1%, P=0.001) (FIG. 15). We also studied immunophenotypes of splenic T cells from post-IRI mice. Splenic CD4⁺ and CD8⁺ T cells from the JHU083 treated mice showed increased naïve phenotypes with low CD44 and high CD62L expression (FIG. 21). pS6 and HKII were enhanced in kidney T cells from the JHU083-treated mice, compared to the vehicle-treated mice (FIG. 22). The levels of the other metabolic enzymes were comparable between groups.

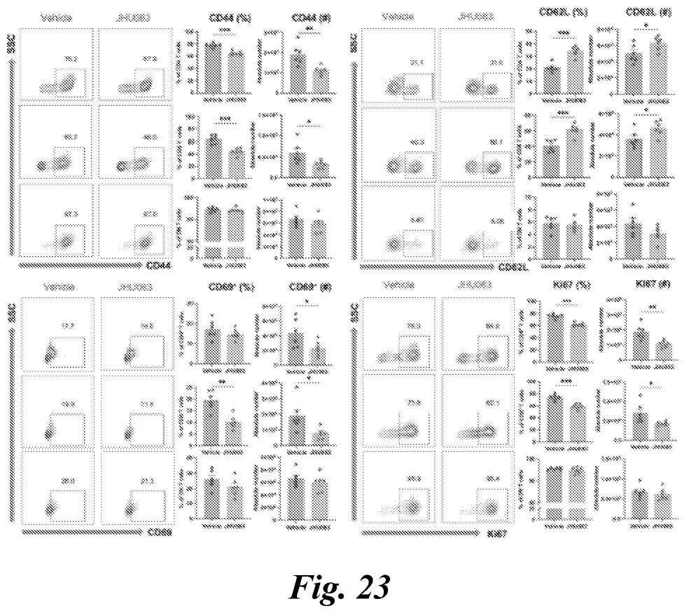

To test whether this immunologic effect was derived from the glutamine blockade or attenuated kidney injury, we studied normal mice treated with JHU083 or vehicle with the same dosage schedule. We found that CD4⁺ and CD8⁺ T cells in steady-state kidneys also were skewed toward naïve phenotypes with lower CD69, CD44, and Ki67 and higher CD62L following glutamine blockade (FIG. 23). Therefore, changes seen in these markers in post-ischemic kidneys are likely through JHU083-mediated glutamine blockade.

### 2.3.8 Glutamine Blockade Exhibited Protective Effect and Reduced T Cell Activation and Proliferation in Nephrotoxic AKI

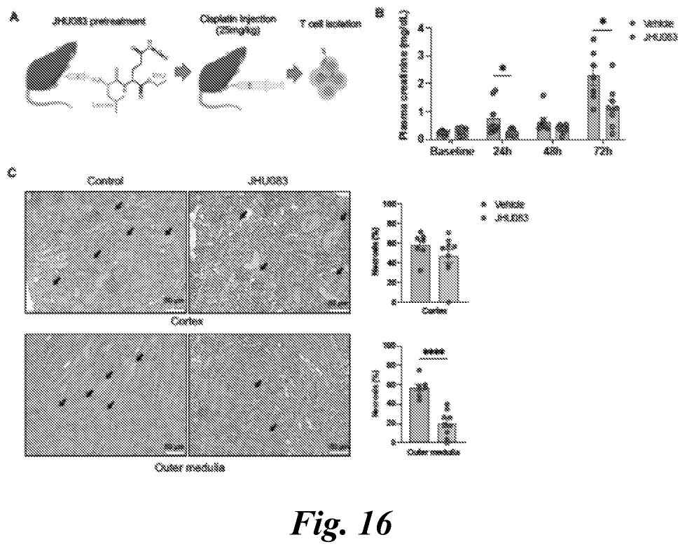

Given the significant protective outcomes in ischemic AKI, we subsequently tested the effect of glutamine blockade on a nephrotoxic AKI model, which is another common etiology of clinical AKI. After pretreatment with JHU083 or vehicle, 25 mg/kg of cisplatin was injected intraperitoneally. T cells were isolated and studied after 72 h after cisplatin treatment (FIG. 16A).

Glutamine blockade with JHU083 treatment reduced functional (plasma creatinine at 72 h, 2.28±0.34 vs 1.17±0.24 mg/dL, P=0.016) and structural renal injury (cortical necrotic tubules, 58.1±4.9 vs 20.9±7.0%, P=0.243, medullary necrotic tubules, 57.1±3.7 vs 19.9±4.5%, P<0.001) in cisplatin-induced AKI (FIG. 16B and FIG. 16C).

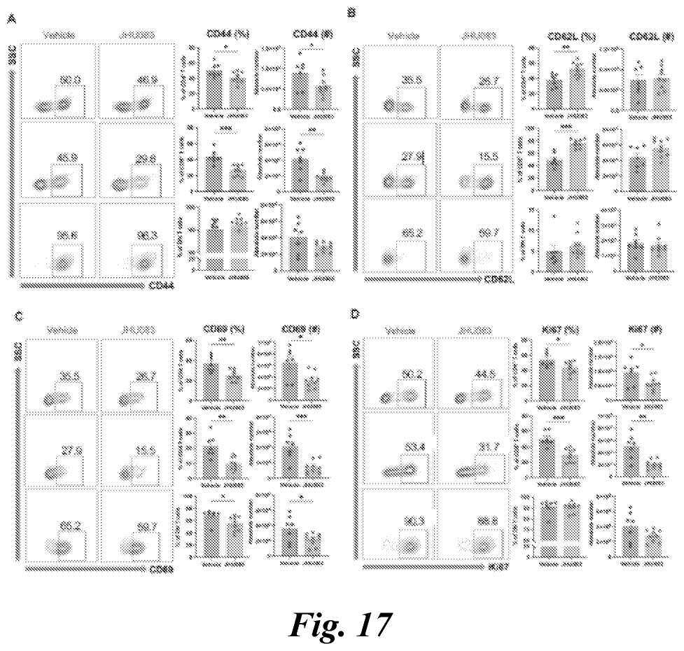

Consistent with immunophenotypic findings in ischemic AKI, JHU083 treated post-cisplatin AKI kidneys had CD4⁺ (CD44⁺, 50.2±3.4 vs 40.9±2.4%, P=0.037; CD62L⁺, 37.5±3.2 vs 52.5±3.0%) and CD8⁺ T cells (CD44⁺, 43.3±3.3 vs 26.4±2.3%, P<0.001; CD62L⁺, 48.9±4.2% vs 76.0±2.3%, P<0.001) with primarily naïve phenotypes. JHU083 treatment reduced activation and proliferation of kidney CD4⁺ (CD69⁺, 37.0±2.9 vs 25.4±1.8%, P=0.014; P=0.005; Ki67⁺, 37.0±2.9 vs 25.4±1.8, P=0.003) and CD8⁺ T cells (CD69⁺, 21.4±2.7 vs 9.8±1.5%, P=0.001; Ki67⁺, 21.4±2.7 vs 9.8±1.5%, P=0.001) (FIG. 17).

### 2.3.9 Glutamine Blockade Inhibited T Cell Activation and Proliferation Under Hypoxia

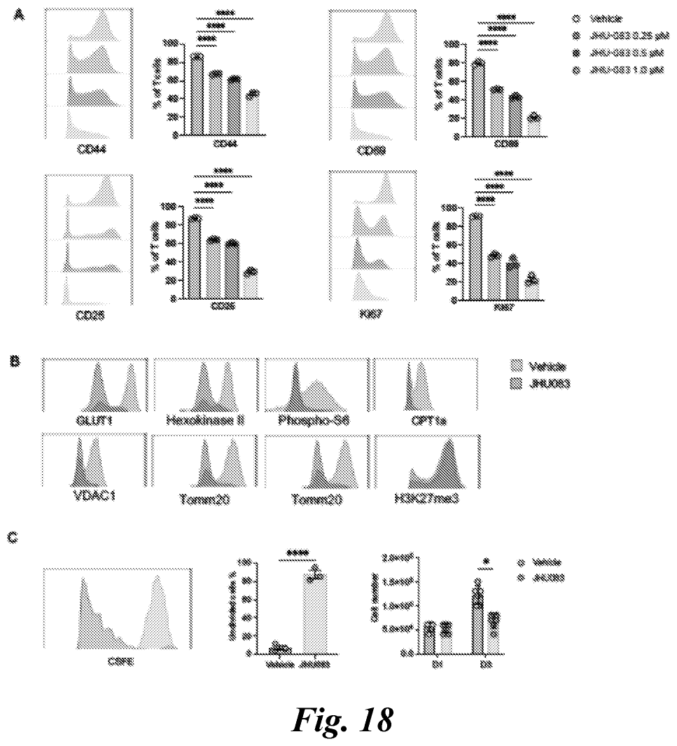

In vitro studies were performed to evaluate the effect of glutamine blockade on activated T cells under hypoxia followed by reoxygenation. Splenic T cells (CD45⁺, TCR αβ⁺) were isolated and cultured using media containing 0.25 μM, 0.5 μM, or 1 μM of JHU083 or vehicle. After 24 h from the culture under CD3/CD28 stimulation, cells were exposed to hypoxia for 24 h followed by reoxygenation. While the majority of vehicle-treated T cells expressed CD44, CD69, CD25, and Ki67, indicating activation and proliferation induced by CD3/CD28, JHU083 treated T cells showed reduced expression of activation and proliferation markers compared to the vehicle-treated cells in a dose-dependent manner (FIG. 18A). Metabolic profiles of JHU083 treated T cells exhibited unstimulated T cell phenotypes, suggesting that glutamine blockade inhibits CD3/CD28 mediated T cell activation in vitro (FIG. 18B).

Glutamine blocking effect on the kidney T cell proliferation was assessed with carboxyfluoroscein succinimidyl ester (CFSE) analysis. FACS sorted kidney T cells (CD45⁺, TCR αβ⁺) were cultured with media containing 1 μM JHU083 or vehicle and underwent hypoxia followed by reoxygenation. There was a significantly higher proportion of undivided cells in the JHU083 treated cells (P<0.001). The cell numbers were significantly lower on day 3 (FIG. 18C).

### 2.4 Discussion

Unlike traditional concepts of adaptive immunity as relatively late responders during inflammation, it is now well established that T cells traffic into kidneys at very early point after AKI and regulate early injury responses. Ascon et al., 2006; Lai et al., 2007. There has been a large body of work in both kidneys and other solid organs demonstrating an important role for T cells in acute tissue injury. Gharaie Fathabad et al., 2020; Yang et al., 2006; Yilmaz et al., 2006; Zwacka et al., 1997.

Given these roles for T cells in AKI and emerging data on importance of T cell metabolism for their function, without wishing to be bound to any one particular theory, it was thought that T cells undergo metabolic reprogramming during AKI and modulation of T cell metabolism could modify AKI outcome. We first performed a descriptive survey with spectral flow cytometry and used machine learning programs to demonstrate that metabolic changes in kidney and splenic T cells after mouse AKI. We identified a distinct T cell subset, unique to post-AKI kidneys, exhibiting reduced mTOR activity and OXPHOS and fatty acid oxidation machineries, but maintaining glycolysis. Furthermore, an early T cell epigenetic modification was found during ischemia. We then studied human kidney to demonstrate clinical relevance of the mouse findings. We then embarked on therapeutic and mechanistic studies targeting T cell metabolism with the glutamine antagonist JHU083. JHU083 was protective in both ischemic and nephrotoxic models of AKI, and changed kidney CD4⁺ and CD8⁺ T cells toward naïve phenotypes. In vitro studies demonstrated that hypoxia induced upregulation of glycolysis in kidney T cells, and glutamine blockade reduced their proliferation.

Spectral flow cytometry overcomes limited multiplexing capacity of fluorescence-based flow cytometry and allows highly complex panel, thus data from spectral flow cytometry are amenable to high-dimensional analyses. Saeys et al., 1016. We also utilized unsupervised machine learning algorithm to discover metabolic reprogramming of T cells. This computational approach provides data visualization and also facilitates identification of unexpected cells or previously undefined cell population for downstream analyses. Tough et al., 2020. Given the recent introduction of spectral flow cytometers, multidimensional computational analysis is becoming increasingly used in immunology research. We predicted that combining metabolic markers with utilizing computational analyses beyond the conventional immunophenotypic markers would provide a deeper understanding of kidney immune cells across the various types of kidney disease.

We found that the metabolic reprogramming of T cells occurred during the very early stage of AKI within 4 h of reperfusion. We demonstrated that expression of histone methylation marker on T cells regulated by TCA cycle was increased during ischemia. This epigenetic modification drives T cell metabolic reprogramming, and the bidirectional relationship between epigenetic imprinting and T cell metabolism is a recently emerging area. Tough et al., 2020. Importantly, we found that T cells maintained glycolysis machinery, whereas OXPHOS and fatty acid oxidation related enzymes were downregulated. VDAC1 is the most prevalent protein in the mitochondrial outer membranes and functions as a major transporter of metabolites. Shoshan-Barmatz et al., 2010. Decreased VDAC1 expression is likely to indicate decreased mitochondrial mass, suggesting mitochondrial dysfunction in AKI. Since mTOR activity is known to be inhibited under cellular hypoxia and lack of nutrients, Powell et al., 2012, low mTOR activity may represent the consequence of hypoxic stress, leading to a decrease in biosynthesis. Functional relevance of this metabolic changes need to addressed in the future.

In contrast to kidney T cells during AKI, splenic T cells did not undergo hypoxia followed by reperfusion or direct antigen exposure during ischemic AKI. Splenic T cells, however, still exhibited metabolic reprogramming following experimental AKI. Metabolic machineries were globally upregulated in post-AKI spleens, resembling metabolic signatures of activated T cells by viral infection. This finding is in line with previous studies demonstrating extrarenal T cell activation in AKI. Burne-Taney et al., 2006; Xu et al., 2016. Accumulating evidence suggests that this distant immunologic effect of AKI is associated with multiorgan dysfunction in AKI, affecting patients' overall outcome and mortality. Lee et al., 2018. Metabolic reprograming of extrarenal T cells followed by AKI from our data highlights T cell-mediated distant organ crosstalk in AKI, emphasizing its importance as a potential therapeutic target. The underlying mechanism which induces splenic T cell metabolic reprogramming in AKI needs to be further explored.

Our in vitro data demonstrated that hypoxia per se was capable of inducing metabolic rewiring of kidney T cells with increased glycolysis. This upregulation of glycolysis following hypoxia exposure has been demonstrated by a previous study using T cells from lymphoid organs. Xu et al., 2016. The metabolic landscape of kidney T cells under in vitro hypoxia, however, was different from the in vivo findings that showed more complex signatures. One possible reason for this difference is cell culture media contains much higher concentration of nutrients than those present in the harsh metabolic environment within tissue. Cantor, 2019.

T cell activation is a glutamine dependent process, and TCR stimulation signal mediates glutamine uptake in naïve T cells. Nakaya et al., 2014; Carr et al., 2010. Activated inflammatory T cells show 5- to 10-fold increase in glutamine uptake. Other amino acids are unable to replace glutamine because transport capacity of other amino acid on T cells is insufficient to compensate glutamine utilization. Carr et al., 2010. Therefore, based on exceptionally high glutamine demands of the effector T cells, we induced metabolic reprogramming with glutamine blockade.

Achieving in vivo glutamine antagonism was previously limited because of dose limiting gastrointestinal toxicity of conventional glutamine antagonist DON. Lemberg et al., 2018. A prodrug strategy was used to change pharmacokinetic profiles and reduce toxicity. Rautioi et al., 2018. We therefore utilized the recently developed prodrug form of DON, JHU083, designed to increase biosafety and inhibit a broad range of glutamine-requiring reactions. Lemberg et al., 2018. We demonstrated that glutamine antagonism significantly improved functional and structural renal outcomes in both ischemic and nephrotoxic models of AKI. JHU083 prevented kidney CD4⁺ and CD8⁺ T cell activation, steering these cells to a naïve-like phenotype with low proliferative capacity. Thus, the mechanism by which glutamine antagonism has a protective effect in experimental AKI could be attributed to inhibiting effector functions of CD4⁺ T cells. We cannot rule out important effects on other immune or non-immune cells, however, such as renal tubular epithelial cells.

Targeting metabolism to regulate immune response is a rapidly evolving area. Patel et al., 2019. Our findings are consistent with previous studies, which have shown beneficial effects of glutamine antagonism in infections, autoimmune diseases and alloimmunity models. Lee et al., 2015; Hollinger et al., 2019; Johnson et al., 2018; Takahashi et al., 2017. Given that we found that glutamine antagonism provided a protective effect on cisplatin-induced AKI, and the published antitumor effect of glutamine antagonism, Oh et al., 2020, combining glutamine blocking strategy to cisplatin-based chemotherapy regimens could be a promising approach to enhance antineoplastic activity of cisplatin while also providing renoprotection.

Despite the low selectivity of drugs that target metabolism, cellular selectivity can be achieved through cellular metabolic demands and programs. Lee et al., 2015. While ordinary cells have more flexible metabolism, effector CD4⁺ and CD8⁺ T cells have the greatest demand for glutaminolysis. Thus, glutamine antagonism can selectively inhibit those cells, not altering normal cellular homeostatic function. We found that DN T cells, a recently emerging kidney T cell subset that has shown a protective effect on AKI, Gong et al., 2020; Sadasivam et al., 2019; Martina et al., 2016; Newman-Rivera et al., 2022, were resistant to glutamine blockade, maintaining their activation marker expression and high proliferation capacity. This observation suggests that DN T cells rely on other metabolic machinery rather than glutaminolysis, a topic of further investigation. Although it is known that tubular epithelial cells also are one of the most energy-demanding cells during kidney injury, they rely on fatty acid oxidation as a major fuel source, unlike effector T cells. Kang et al., 2015; Miguel et al., 2021.

Although strategies targeting immune reconstitution to suppress T cell-mediated inflammation in AKI has been tested previously with conventional immunosuppressive drugs, mTOR inhibitor or mycophenolate mofetil, they failed to show a protective effect. Liu et al., 2009; Gandolfo et al., 2010. The negative outcome was associated with inadvertent suppression of regulatory cell subsets that have a protective role in AKI. Liu et al., 2009; Gandolfo et al., 2010. Given this finding, instead of suppressing whole immunity, selective suppression of pathogenic effector T cell subsets appears to be important to achieve a protective effect. Therefore, targeting a metabolic pathway that is selectively required for pathogenic T cells might be a useful strategy to improve AKI outcomes.

Glutamine supplementation, however, has been shown to have a contrasting protective role in some studies that used different experimental AKI models, including septic, myoglobinuric, and folic acid induced AKI. Kim et al., 2009; Hu et al., 2012; Peng et al., 2013. We postulate that these discordant findings may be due to distinct disease mechanisms per different AKI models. A large multicenter clinical trial demonstrated that parenteral glutamine supplementation was associated with increased mortality and hospital stay, especially in patients with renal dysfunction, but the mechanisms remain uncertain. Heyland et al., 2013; Heyland et al., 2015. A few studies that addressed immunologic effects showed that glutamine supplementation was associated with enhanced Th1 polarization, reduced circulating Tregs, and increased renal IL-1 and IL-6 expression. Alba-Loureiro et al., 2010; Hou et al., 2019. Future studies will be required to understand the underlying mechanism of negative clinical outcomes associated with glutamine supplementation.

The current study has several limitations. First, although we focused on T cells which play important roles in AKI pathogenesis, other types of immune cells in kidneys also are involved in AKI pathogenesis. Jang et al., 2015. Therefore, future studies addressing metabolism of non-T cell populations such as neutrophils, mononuclear phagocytic cells, B cells, or innate lymphoid cells are warranted. Thus, we cannot rule out a collateral effect of JHU083 on other types of immune cells in kidneys, or even non-immune epithelial cells. Second, to achieve T cell metabolic reprogramming, we pharmacologically targeted glutamine-related pathways because of a key role in T cell metabolism, the availability of JHU083 and safety in cancer models—but there are metabolic inhibitors that target other metabolic pathways. Corrado and Pearce, 2022. Further studies using different inhibitors targeting non-glutamine pathways or combinations with glutamine blockade will provide a deeper understanding of T cell metabolism in AKI. Since T cells play important roles in renal recovery and repair, as well as early injury, Gharaie Fathabad et al., 2020, suppressing T cell-mediated inflammatory process may inadvertently disrupt the regeneration and recovery process. Rabb et al., 2016. Since our study focused on the early injury phase and local tissue damage, effects of glutamine blockade on long-term renal effects and distant organ effects need to be investigated. Another limitation was that we did not directly measure cellular metabolites, but measured enzymes and receptors involved in metabolic pathways. Measuring cellular metabolites directly using biochemical approaches, such as mass spectrometry, can provide detailed information for each metabolic pathway. Alseekh et al., 2021. A caveat of this technique, however, is that this measure provides average metabolomics of whole tissue, not specific cellular insight. Given the uniqueness of T cell metabolic programming, Patel et al., 2019, metabolic profiles of whole kidney extracts may differ from T cell metabolic profiles. It also should be noted that human data were limited to preischemic and ischemic nephrectomized kidneys because there was no reperfusion in nephrectomy samples like the mouse IRI model. Future studies also will need to be performed going into more depth on the precise mechanisms of JHU083 effects on kidney T cell populations.

Despite these and other limitations, our study is novel has important future scientific and clinical implications. We demonstrated the immune-metabolic phenotyping changes of kidney T cells in mice and humans at baseline and during AKI. This approach has implications for non-AKI diseases of the kidney ranging from glomerulonephritis to allograft rejection. Furthermore, our findings are relevant for acute ischemic and toxic injury to other organs like liver, heart and brain. We also demonstrated that pharmacologic intervention targeting glutamine in T cell metabolism can improve both ischemic and nephrotoxic AKI outcomes, with human translational potential.

### 2.5 Methods

### 2.5.1 Mice

Seven-week-old male C57BL/6J wild-type (WT) mice were purchased from Jackson Laboratory (Bar Harbor, ME) and bred under specific pathogen-free condition at the Johns Hopkins University animal facility. C57BL/6J WT mice were infected with LCMV Armstrong virus (2×10⁵ pfu/mouse, i.p.) kindly provided by Susan Kaech. Organs were collected on day 7 at peak of acute infection.

### 2.5.2 Ischemic AKI Model

Mice were anesthetized with an intraperitoneal injection of pentobarbital (75 mg/kg; Akorn, Lake Forest, IL). After shaving of abdominal hair, mice were placed onto a thermostatically controlled heating table. Abdominal midline incision was performed, and both renal pedicles were dissected and clamped for 30 min with a microvascular clamp (Roboz Surgical Instrument, Gaithersburg, MD). After 30 min, microvascular clamps were released from renal pedicles and the kidneys were inspected to confirm reperfusion. During the surgery, mice were kept well hydrated with 1 mL of warm sterile 0.9% saline. After being sutured, mice were allowed to recover with free access to chow and water. Sham surgeries were performed identically without clamping of renal pedicles.

### 2.5.3 Cisplatin AKI Model

Cisplatin (cis-diammineplatinum II dichloride; Sigma-Aldrich, St. Louis, MO) was freshly dissolved into 0.9% saline (1 mg/mL) on the day of injection. The dissolved solution was incubated in water bath at 40° C. for 10 min to achieve complete dissolution. A single 25 mg/kg dose of cisplatin was injected intraperitoneally.

### 2.5.4 Assessment of Kidney Function

Blood samples were obtained at 0, 24, 48, and 72 h after IRI or cisplatin injection through tail vein collection. Plasma creatinine concentration was measured by Cobas Mira Plus automated analyzer system (Roche, Indianapolis, IN) with creatinine reagent (Pointe Scientific Inc, Canton, MI).

### 2.5.5 Tissue Histological Analysis

At 24 h after IRI and 72 h after cisplatin injection, mice were anesthetized with ketamine (130 mg/kg; VetOne, Boise, ID) and xylazine (7 mg/kg; Akorn) intraperitoneal injection. After exsanguination, kidneys were collected and fixed with 10% buffered formalin followed by paraffin embedding. Tissue sections were stained with hematoxylin and eosin. A renal pathologist scored necrotic tubules in a blinded fashion.

### 2.5.6 Isolation of Kidney Mononuclear Cells and Splenocytes

KMNCs were isolated according to our previously described Percoll density gradient protocol. Ascon et al., 2006. Briefly, decapsulated kidneys were immersed and incubated in collagenase D (2 mg/mL; Sigma-Aldrich) solution for 30 min at 37° C. Samples were strained through 70 m cell strainer (BD Bioscience, Franklin Lakes, NJ), washed, and resuspended in 40% Percoll (GE Healthcare, Chicago, IL) followed by gentle overlaying onto 80% Percoll. After centrifugation at 1,500 g for 30 min in brake off mode at room temperature, KMNCs were collected from interface between 40% and 80% Percoll. Spleens were strained through 40 m cell strainer (BD Bioscience) and incubated with ammonium-chloride-potassium lysis buffer (Quality Biological, Gaithersburg, MD) for 3 min. Collected cells were washed and resuspended with Roswell Park Memorial Institute (RPMI) 1640 media (Thermo Fisher Scientific, Waltham, MA) containing 5% fetal bovine serum (FBS, Thermo Fisher Scientific). Cells were counted on hemocytometer using trypan blue exclusion under a microscope.

### 2.5.7 Isolation of Human Kidney Mononuclear Cells

Human kidney tissues were procured from normal portions of renal cell carcinoma nephrectomy kidneys. Pre-ischemic kidney tissues were obtained before clamping of renal hilum, whereas ischemic kidney tissues were obtained after clamping of renal hilum.

Ischemia time at body temperature for ischemic kidney tissues was 35 to 45 min. The obtained kidney tissues were immediately kept on ice and digested according to the above-described protocol. KMNCs were viably cryopreserved in FBS with 10% Dimethyl sulfoxide (DMSO; Thermo Fisher Scientific, Waltham, MA) for downstream analyses.

### 2.5.8 Spectral Flow Cytometry

Cells were washed once with phosphate buffered saline (PBS) and stained with viability dye Zombie NIR Fixable Viability (BioLegend, San Diego, CA) for 15 min at room temperature. After washing with Cell Staining Buffer (BioLegend), cells were preincubated with anti-CD16/CD32 Fc receptor blocking antibody (S17011E, BioLegend) for 15 min to prevent nonspecific antibody binding. Subsequently, surface staining was performed in 50 μL of BD horizon™ Brilliant Stain buffer and surface staining antibody cocktail for 30 min at 4° C.: Pacific blue anti-CD44 (IM7, BioLegend), BV510 anti-CD8 (53-6.7, BioLegend), BV570 anti-CD45 (30-F11, BioLegend), BV605 anti-CD69 (H1.2F3, BioLegend), BV650 anti-NK1.1 (PK136, BioLegend), BV711 anti-PD1 (29F.1A12, BioLegend), BV785 anti-TCRβ (H57-597, BioLegend), PE-Cy5.5 anti-CD25 (PC61.5, Thermo Fisher Scientific), APC-R700 anti-CD62L (MEL-14, BD Bioscience), and APC-Fire810 anti-CD4 (GK1.5, BioLegend). Cells were fixed and permeabilized with Foxp3/Transcription Factor Staining kit (Thermo Fisher Scientific) for 30 min at room temperature and washed with permeabilization/wash buffer (Thermo Fisher Scientific). Intracellular staining was conducted in 50 μL of permeabilization/wash buffer with intracellular staining antibody cocktail for 45 min at room temperature: BV421 anti-Ki67 (16A8, BioLegend), Alexa Fluor 488 anti-CPT1a (8F6AE9, Abcam, Cambridge, UK), Alexa Fluor 532 anti-VDAC1 (20B12AF2, Abcam), PerCP-efluor 710 anti-FoxP3 (FJK-16S, Thermo Fisher Scientific), PE anti-GLUT1 (EPR3915, Abcam), Alexa Fluor 594 anti-pS6 (D68F8, Cell Signaling, Danvers, MA), PE-Cy5 anti-HKII (EPR20839, Abcam), PE-Cy7 anti-H3K27me3 (C36B11, Cell Signaling), and Alexa Fluor 647 anti-Tomm20 (EPR15581-54, Abcam). After staining, cells were washed with permeabilization/wash buffer then resuspended in Cell Staining Buffer. Samples were analyzed by 4-laser Aurora spectral flow cytometer (Cytek, Fremont, CA).

For the human kidney, cryopreserved KMNCs were thawed and washed with PBS. Cells were stained for viability assay with Zombie NIR for 15 min at room temperature. After preincubation with anti-CD16/CD32, cells were stained as described above with following fluorochrome-labeled antibodies for surface and intracellular staining: BV786 anti-CD3 (SK7, BD Biosciences), BV480 anti-CD8 (RPA-T8, BD Biosciences), BV570 anti-CD45RA (HI100, BioLegend), BV650 anti-CCR7 (G043H7, BioLegend), BV510 anti-CD25 (M-A251, BD Biosciences), BV711 anti-PD1 (EH12.2H7, BioLegend), PE-Cy5 anti-CD4 (OKT4, BioLegend), PE-Cy5.5 anti-CD69 (CH/4, ThermoFisher Scientific), APC anti-CD49a (TS2/7, BioLegend), APC-Cy7 anti-CD19 (SJ25C1, BioLegend), APC-Cy7 anti-CD56 (5.1H11, BioLegend), Pacific Blue anti-FoxP3 (206D, BioLegend), Alexa Fluor 405 anti-Tomm20 (EPR15581-54, Abcam), AF532 anti-VDAC1 (20B12AF2, Abcam), Alexa Fluor 488 anti-CPT1a (8F6AE9, Abcam), PE H3K27me3 (C36B11, Cell Signaling Technology), Alexa Fluor 680 anti-HKII (EPR20839, Abcam), and Alexa Fluor 647 GLUT1 (EPR3915, Abcam).

### 2.5.9 High-Dimensional Flow Cytometry Data Analysis

The acquired raw data from the spectral flow cytometer were unmixed by SpectroFlo software (Cytek). Unmixed data was first curated with FlowJo 10.8 software (BD Biosciences) to remove debris, doublets, and dead cells. Curated data were downsampled and concatenated to conduct downstream analyses. High-dimensional unbiased analyses were performed using FlowJo plugin UMAP 3.1.

### 2.5.10 In Vivo Glutamine Blockade

Glutamine antagonist, JHU083 was synthesized as previously described, Rais et al., 2016, and dissolved in 50 mM 10-μM 4-(2-hydroxyethyl)-1-piperazineethanesulfonic acid (HEPES) buffered 0.9% saline. Aliquot stocks were stored at −80° C. and thawed right before injection. Before the IRI surgery or cisplatin injection, mice were administered 1.83 mg/kg JHU083 (equivalent to 1 mg/kg DON) or vehicle (50 mM HEPES buffered 0.9% saline) every other day via intraperitoneal injection. Following 4 consecutive dosages, mice underwent IRI surgery or cisplatin injection. The last additional dose was given at 24 h after IRI or cisplatin injection. This dosage regimen was chosen based on efficiency and toxicity data from a previous study. Hollinger et al., 2020. Body weight was monitored every other day and no significant differences between groups were observed due to JHU083 treatment.

### 2.5.11 Kidney and Splenic T Cell Isolation for Cell Culture

Kidney T cells were isolated using FACS. Briefly, single-cell suspension of KMNCs was preincubated with anti-CD16/CD32 Fc block (S17011E, BioLegend) stained in Cell Staining Buffer (BioLegend) with fluorochrome-labeled antibodies: APC-Cy7 anti-CD45 (30-F11) and BV421 anti-TCRβ (H57-957) from BioLegend. Live Dead Aqua (Thermo Fisher Scientific) was stained for viability assay. Live Dead Aqua CD45⁺ TCRβ⁺ cells were sorted with FACSAria II Cell Sorter (BD Bioscience). Splenic T cells were isolated from single cells suspension of spleens using a T Cell Isolation Kit II (Miltenyi Biotec, North Rhine-Westphalia, Germany) according to the manufacturer's guideline.

### 2.5.12 In Vitro T Cell Culture

48-well flat-bottom plates were coated with 5-μg/mL anti-CD3 in PBS and incubated at 4° C. overnight. Anti-CD3 coated plates were washed with PBS, and isolated cells were cultured in RPMI 1640 GlutaMAX (Thermo Fisher Scientific) supplemented with 10% FBS (Thermo Fisher Scientific), 10 μM HEPES buffer (Thermo Fisher Scientific), 100-μM non-essential amino acid solution (Sigma-Aldrich), and 55-μM 2-mercaptoethanol (Sigma-Aldrich)±JHU083. After 72 hours of culture, cells were stained with flow cytometry antibodies as previously described.

### 2.5.13 In Vitro Hypoxia Induction

Cultured cells were incubated under hypoxic (1% O₂) condition according to the following protocol. For hypoxia induction, culture plates were placed in a modular incubator chamber, and the chamber was flushed with gas mixture containing 1% O₂, 5% CO₂, and 94% N₂ for 3 min. After flushing the incubator was completely sealed and placed into cell culture incubator for 24 h.

### 2.5.14 Glutaminase Activity Analysis

Glutaminase activity measurements in kidney samples were adapted from previously described protocols. Engler et al., 2002. Briefly, kidneys were homogenized using Biomasher II and then sonicated (three pulses of 15 s duration on ice using Kontes' Micro Ultrasonic Cell Disrupter) in ice-cold potassium phosphate buffer (45 mM, pH 8.2) containing protease inhibitors (Roche, Complete Protease Inhibitor Cocktail, 1 tablet in 10 mL) and incubated with [³H]-glutamine (0.04 μM, 0.91 μCi) for 90 min at room temperature. The reactions were carried out in 50 μL reaction volumes in a 96-well microplate. At the end of the reaction period, the assay was terminated upon the addition of imidazole buffer (20 mM, pH 7). 96-well spin columns packed with strong anion ion-exchange resin (Bio-Rad, AG® 1-X2 Resin, 200-400 mesh, chloride form) were used to separate the substrate and the reaction product. Unreacted [³H]-glutamine was removed by washing with imidazole buffer. [³H]-Glutamate, the reaction product, was then eluted with 0.1 N HCl and analyzed for radioactivity using Perkin Elmer's TopCount instrument in conjunction with their 96-well LumaPlates. Finally, total protein measurements were carried as per manufacturer's instructions using BioRad's Detergent Compatible Protein Assay kit and data are presented as fmol/mg/h.

### 2.5.15 Statistics

Data were expressed as mean±standard error of mean (SEM). Groups means were compared with T test or ANOVA followed by Tukey's post-hoc analyses using GraphPad Prism version 9 (GraphPad Software, La Jolla, CA). P values <0.05 were considered statistically significant.

### 2.5.16 Study Approval

The human study was performed in accordance with the Declaration of Helsinki and approved by the Johns Hopkins Medicine Institutional Review Board (CR00031498). Written informed consent was received prior to participation, and all data were deidentified.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Kellum J A, et al. Acute kidney injury. Nat Rev Dis Primers. 2021; 7(1):52.
Aufhauser D D, Jr., et al. Improved renal ischemia tolerance in females influences kidney transplantation outcomes. J Clin Invest. 2016; 126(5):1968-1977.
Bonventre J V, and Yang L. Cellular pathophysiology of ischemic acute kidney injury. J Clin Invest. 2011; 121(11):4210-4221.
Jang H R, and Rabb H. Immune cells in experimental acute kidney injury. Nat Rev Nephrol. 2015; 11(2):88-101.
Wei Q, et al. MicroRNA-668 represses MTP18 to preserve mitochondrial dynamics in ischemic acute kidney injury. J Clin Invest. 2018; 128(12):5448-5464.
Yang L, et al. Epithelial cell cycle arrest in G2/M mediates kidney fibrosis after injury. Nat Med. 2010; 16(5):535-543, 531p following 143.
Ying W Z, et al. Immunoglobulin light chains generate proinflammatory and profibrotic kidney injury. J Clin Invest. 2019; 129(7):2792-2806.
Burne M J, et al. Identification of the CD4+ T cell as a major pathogenic factor in ischemic acute renal failure. J Clin Invest. 2001; 108(9):1283-1290.
Rabb H, et al. Pathophysiological role of T lymphocytes in renal ischemia-reperfusion injury in mice. Am J Physiol Renal Physiol. 2000; 279(3):F525-531.
Pearce E L, et al. Fueling immunity: insights into metabolism and lymphocyte function. Science. 2013; 342(6155):1242454.
Buck M D, et al. Metabolic Instruction of Immunity. Cell. 2017; 169(4):570-586.
Baumann T, et al. Regulatory myeloid cells paralyze T cells through cell-cell transfer of the metabolite methylglyoxal. Nat Immunol. 2020; 21(5):555-566.
Buck M D, et al. Mitochondrial Dynamics Controls T Cell Fate through Metabolic Programming. Cell. 2016; 166(1):63-76.
Patel C H, et al. Targeting metabolism to regulate immune responses in autoimmunity and cancer. Nat Rev Drug Discov. 2019; 18(9):669-688.
Teng X, et al. Immune cell metabolism in autoimmunity. Clin Exp Immunol.
2019; 197(2):181-192.
Sears J D, et al. Targeting metabolism to reverse T-cell exhaustion in chronic viral infections. Immunology. 2021; 162(2):135-144.
Zou Y, and Chen B J. T cell metabolism in graft-versus-host disease. Blood Science.
2020; 2(1):16-21.
Ascon D B, et al. Phenotypic and functional characterization of kidney-infiltrating lymphocytes in renal ischemia reperfusion injury. J Immunol. 2006; 177(5):3380-3387.
Lai L W, et al. A sphingosine-1-phosphate type 1 receptor agonist inhibits the early T-cell transient following renal ischemia-reperfusion injury. Kidney Int. 2007; 71(12):1223-1231.
Oh M H, et al. Targeting glutamine metabolism enhances tumor-specific immunity by modulating suppressive myeloid cells. J Clin Invest. 2020; 130(7):3865-3884.
Bettencourt I A, and Powell J D. Targeting Metabolism as a Novel Therapeutic Approach to Autoimmunity, Inflammation, and Transplantation. J Immunol. 2017; 198(3):999-1005.
Lee C F, et al. Preventing Allograft Rejection by Targeting Immune Metabolism. Cell Rep. 2015; 13(4):760-770.
Leone R D, et al. Glutamine blockade induces divergent metabolic programs to overcome tumor immune evasion. Science. 2019; 366(6468):1013-1021.
Thompson E A, et al. Metabolic programs define dysfunctional immune responses in severe COVID-19 patients. Cell Rep. 2021; 34(11):108863.
Pearce E L, and Shen H. Making sense of inflammation, epigenetics, and memory CD8+ T-cell differentiation in the context of infection. Immunol Rev. 2006; 211:197-202.
Hotchin J. The contamination of laboratory animals with lymphocytic choriomeningitis virus. Am J Pathol. 1971; 64(3):747-769.
Lee S A, et al. Distant Organ Dysfunction in Acute Kidney Injury: A Review. Am J Kidney Dis. 2018; 72(6):846-856.
Lemberg K M, et al. We're Not “DON” Yet: Optimal Dosing and Prodrug Delivery of 6-Diazo-5-oxo-L-norleucine. Mol Cancer Ther. 2018; 17(9):1824-1832.
Rais R, et al. Discovery of 6-Diazo-5-oxo-1-norleucine (DON) Prodrugs with Enhanced CSF Delivery in Monkeys: A Potential Treatment for Glioblastoma. J Med Chem.
2016; 59(18):8621-8633.
Hollinger K R, et al. Glutamine Antagonist JHU-083 Normalizes Aberrant Hippocampal Glutaminase Activity and Improves Cognition in APOE4 Mice. J Alzheimers Dis. 2020; 77(1):437-447.
Gharaie Fathabad S, et al. T Lymphocytes in Acute Kidney Injury and Repair. Semin Nephrol. 2020; 40(2):114-125.
Yang Z, et al. Myocardial infarct-sparing effect of adenosine A2A receptor activation is due to its action on CD4+ T lymphocytes. Circulation. 2006; 114(19):2056-2064.
Yilmaz G, et al. Role of T lymphocytes and interferon-gamma in ischemic stroke. Circulation. 2006; 113(17):2105-2112.
Zwacka R M, et al. CD4(+) T-lymphocytes mediate ischemia/reperfusion-induced inflammatory responses in mouse liver. J Clin Invest. 1997; 100(2):279-289.
Saeys Y, et al. Computational flow cytometry: helping to make sense of high-dimensional immunology data. Nat Rev Immunol. 2016; 16(7):449-462.
Tough D F, et al. Epigenetic Regulation of T Cell Memory: Recalling Therapeutic Implications. Trends Immunol. 2020; 41(1):29-45.
Shoshan-Barmatz V, et al. VDAC, a multi-functional mitochondrial protein regulating cell life and death. Mol Aspects Med. 2010; 31(3):227-285.
Powell J D, et al. Regulation of Immune Responses by mTOR. Annual Review of Immunology. 2012; 30(1):39-68.
Burne-Taney M J, et al. Transfer of lymphocytes from mice with renal ischemia can induce albuminuria in naive mice: a possible mechanism linking early injury and progressive renal disease? Am J Physiol Renal Physiol. 2006; 291(5):F981-986.
Burne-Taney M J, et al. Persistent renal and extrarenal immune changes after severe ischemic injury. Kidney Int. 2005; 67(3):1002-1009.
Xu Y, et al. Glycolysis determines dichotomous regulation of T cell subsets in hypoxia. J Clin Invest. 2016; 126(7):2678-2688.
Cantor J R. The Rise of Physiologic Media. Trends Cell Biol. 2019; 29(11):854-861.
Nakaya M, et al. Inflammatory T cell responses rely on amino acid transporter ASCT2 facilitation of glutamine uptake and mTORC1 kinase activation. Immunity.
2014; 40(5):692-705.
Carr E L, et al. Glutamine uptake and metabolism are coordinately regulated by ERK/MAPK during T lymphocyte activation. J Immunol. 2010; 185(2):1037-1044.
Rautio J, et al. The expanding role of prodrugs in contemporary drug design and development. Nat Rev Drug Discov. 2018; 17(8):559-587.
Hollinger K R, et al. Glutamine antagonism attenuates physical and cognitive deficits in a model of MS. Neurol Neuroimmunol Neuroinflamm. 2019; 6(6).
Johnson M O, et al. Distinct Regulation of Th17 and Th1 Cell Differentiation by Glutaminase-Dependent Metabolism. Cell. 2018; 175(7):1780-1795.e1719.
Takahashi S, et al. Glutaminase 1 plays a key role in the cell growth of fibroblast-like synoviocytes in rheumatoid arthritis. Arthritis Research & Therapy. 2017; 19(1):76.
Gong J, et al. TCR(+)CD4(−)CD8(−) (double negative) T cells protect from cisplatin-induced renal epithelial cell apoptosis and acute kidney injury. Am J Physiol Renal Physiol. 2020; 318(6):F1500-F1512.
Sadasivam M, et al. Activation and Proliferation of PD-1(+) Kidney Double-Negative T Cells Is Dependent on Nonclassical MHC Proteins and IL-2. J Am Soc Nephrol. 2019; 30(2):277-292.
Martina M N, et al. Double-Negative alphabeta T Cells Are Early Responders to AKI and Are Found in Human Kidney. J Am Soc Nephrol. 2016; 27(4):1113-1123.
Newman-Rivera A M, et al. TCRαβ+ CD4−/CD8− “double negative” T cells in health and disease-implications for the kidney. Kidney Int. 2022.
Kang H M, et al. Defective fatty acid oxidation in renal tubular epithelial cells has a key role in kidney fibrosis development. Nat Med. 2015; 21(1):37-46.
Miguel V, et al. Renal tubule Cpt1a overexpression protects from kidney fibrosis by restoring mitochondrial homeostasis. J Clin Invest. 2021; 131(5).
Liu M, et al. Effects of delayed rapamycin treatment on renal fibrosis and inflammation in experimental ischemia reperfusion injury. Transplant Proc.
2009; 41(10):4065-4071.
Gandolfo M T, et al. Mycophenolate mofetil modifies kidney tubular injury and Foxp3+ regulatory T cell trafficking during recovery from experimental ischemia-reperfusion. Transpl Immunol. 2010; 23(1-2):45-52.
Kim Y S, et al. Glutamine attenuates tubular cell apoptosis in acute kidney injury via inhibition of the c-Jun N-terminal kinase phosphorylation of 14-3-3. Crit Care Med. 2009; 37(6):2033-2044.
Hu Y M, et al. Glutamine administration ameliorates sepsis-induced kidney injury by downregulating the high-mobility group box protein-1-mediated pathway in mice. Am J Physiol Renal Physiol. 2012; 302(1):F150-158.
Peng Z Y, et al. The anti-oxidant effects are not the main mechanism for glutamine's protective effects on acute kidney injury in mice. Eur J Pharmacol. 2013; 705(1-3):11-19.
Heyland D, et al. A randomized trial of glutamine and antioxidants in critically ill patients. N Engl J Med. 2013; 368(16):1489-1497.
Heyland D K, et al. Glutamine and antioxidants in the critically ill patient: a post hoc analysis of a large-scale randomized trial. JPEN J Parenter Enteral Nutr. 2015; 39(4):401-409.
Alba-Loureiro T C, et al. Effects of glutamine supplementation on kidney of diabetic rat. Amino Acids. 2010; 38(4):1021-1030.
Hou Y-C, et al. Effects of prophylactic administration of glutamine on CD4+ T cell polarisation and kidney injury in mice with polymicrobial sepsis. British Journal of Nutrition. 2019; 122(6):657-665.
Corrado M, and Pearce E L. Targeting memory T cell metabolism to improve immunity. J Clin Invest. 2022; 132(1).
Rabb H, et al. Inflammation in AKI: Current Understanding, Key Questions, and Knowledge Gaps. J Am Soc Nephrol. 2016; 27(2):371-379.
Alseekh S, et al. Mass spectrometry-based metabolomics: a guide for annotation, quantification and best reporting practices. Nat Methods. 2021; 18(7):747-756.
Engler J A, et al. Properties of glutaminase of crayfish CNS: implications for axon-glia signaling. Neuroscience. 2002; 114(3):699-705.

Although the foregoing subject matter has been described in some detail by way of illustration and example for purposes of clarity of understanding, it will be understood by those skilled in the art that certain changes and modifications can be practiced within the scope of the appended claims.

1. A method for preventing or treating a subject afflicted with, suspected of having, or susceptible to having an acute kidney injury, the method comprising administering to the subject at least one glutamine antagonist, or a prodrug or analog thereof, in an amount effective to prevent or treat the acute kidney injury.

2. The method of claim 1, wherein the prodrug of the at least one glutamine antagonist comprises a compound of formula (I): 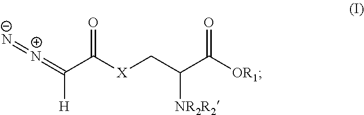
or a pharmaceutically acceptable salt thereof, in an amount effective to treat the acute kidney injury, wherein:
X is selected from the group consisting of a bond, —O—, and —(CH₂)ₙ—, wherein n is an integer selected from the group consisting of 1, 2, 3, 4, 5, 6, 7, and 8;
R₁ and R₂′ together form a 4- to 6-membered heterocylic ring comprising the oxygen atom adjacent to R₁ and the nitrogen atom adjacent to R₂′ or R₁ is selected from the group consisting of H, C₁₋₆ alkyl, and substituted C₁₋₆ alkyl;
R₂ is selected from the group consisting of
i) 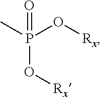
wherein Rₓ and Rₓ′ are each independently selected from the group consisting of alkyl, substituted alkyl, cycloalkyl, substituted cycloalkyl, aryl, substituted aryl, aralkyl, substituted aralkyl, heterocyclyl, substituted heterocyclyl, alkenyl, substituted alkenyl, cycloalkenyl, substituted cycloalkenyl, alkylamine, substituted alkylamine, heteroaryl, and substituted heteroaryl. 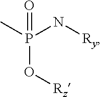
ii) wherein R_{y} and R_{z} are each independently selected from the group consisting of H, alkyl, substituted alkyl, cycloalkyl, substituted cycloalkyl, heterocyclyl, substituted heterocyclyl, alkenyl, substituted alkenyl, cycloalkenyl, substituted cycloalkenyl, —(CR₃R₄)ₘ—Z, —(CR₃R₄)ₘ-Q-Z, aryl, substituted aryl, alkylamine, substituted alkylamine, heteroaryl, substituted heteroaryl, and 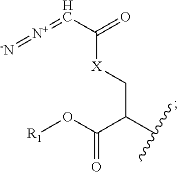
iii) H, alkyl, —C(═O)—Ar, —C(═O)—Y—(CR₃R₄)ₘ—Ar, —C(═O)—Y—(CR₃R₄)ₘ—NR₅R₆, —P(═O)(OR₇)ₙ(NHR₉)ₒ, —C(═O)—Y—(CR₃R₄)ₘ—Ar—O—C(═O)—R₈, —C(═O)—Y—(CR₃R₄)ₘ—Ar—O—R₈, —C(═O)—O—(CR₃R₄)ₘ—O—C(═O)—R₁₀, —C(═O)—O—R₉, —C(═O)—Y—(CR₃R₄)ₘ—Ar—O—C(═O)—Ar, and —C(═O)—Y—(CR₃R₄)ₘ—Ar—NR₅R₆;
wherein: Y is —O— or a bond;
m is an integer selected from the group consisting of 0, 1, 2, 3, 4, 5, 6, 7, and 8;
each n and o is an integer from 0 to 2 provided that the sum of n and o is 2;
R₃ and R₄ are each independently H, C₁-C₆ alkyl or substituted C₁-C₆ alkyl, aryl or substituted aryl, —(CR₃R₄)ₘ—NR₅R₆, or 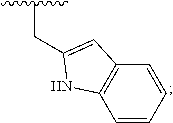 [SMILES: *Cc1cc2ccccc2[nH]1]
each R₅ and R₆ is independently H, alkyl, —C(═O)—(CR₃R₄)ₘ, —C(═O)—(NR₅R₆), or —C(═O)—(CR₃R₄)ₘ—NR₅R₆;
each R₇ is independently selected from the group consisting of H, alkyl, substituted alkyl, cycloalkyl, substituted cycloalkyl, heterocyclyl, substituted heterocyclyl, alkenyl, substituted alkenyl, cycloalkenyl, substituted cycloalkenyl, —(CR₃R₄)ₘ—Z, —(CR₃R₄)ₘ-Q-Z, wherein Q is a monosaccharide, aryl, substituted aryl, heteroaryl, substituted heteroaryl, and wherein Z is 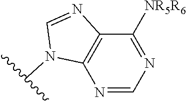
or wherein R₇ together with the oxygen atom to which it is attached forms a purine or pyrimidine nucleoside:
each R₉ is independently selected from the group consisting of H, alkyl, substituted alkyl, cycloalkyl, substituted cycloalkyl, heterocyclyl, substituted heterocyclyl, alkenyl, substituted alkenyl, cycloalkenyl, substituted cycloalkenyl, —(CR₃R₄)ₘ—Z, aryl, substituted aryl, heteroaryl, substituted heteroaryl, and 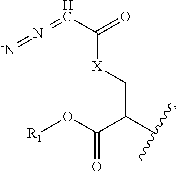
wherein R₁ and X are as defined above, provided that R₁ is not H;
each R₈ is independently alkyl, substituted alkyl, cycloalkyl, substituted cycloalkyl, monosaccharide, acylated monosaccharide, aryl, substituted aryl, heteroaryl, substituted heteroaryl;
each R₁₀ is independently alkyl, substituted alkyl, cycloalkyl, substituted cycloalkyl, monosaccharide, acylated monosaccharide, aryl, substituted aryl, heteroaryl, substituted heteroaryl; and
Ar is aryl, substituted aryl, heteroaryl, or substituted heteroaryl; and
R₂′ is selected from the group consisting of H, C₁-C₆ alkyl, and substituted C₁-C₆ alkyl.

3. The method of claim 2, wherein X is —CH₂—.

4. The method of claim 2, wherein X is —O—.

5. The method of claim 2, wherein R₁ is selected from the group consisting of methyl, ethyl, isopropyl, cyclopentyl, cyclohexyl, trimethylammonium, triethylammonium, tri(hydroxyethyl)ammonium, tripropylammonium, and tri(hydroxypropyl)ammonium.

6. The method of claim 2, wherein:
m is 1;
each R₃ and R₄ are independently H, C₁-C₆ alkyl, aryl or substituted aryl; and
R₁₀ is selected from the group consisting of alkyl, substituted alkyl, cycloalkyl, substituted cycloalkyl, aryl, substituted aryl, heteroaryl, and substituted heteroaryl.

7. The method of claim 2, wherein the compound of formula (I) is a compound having formula (II): 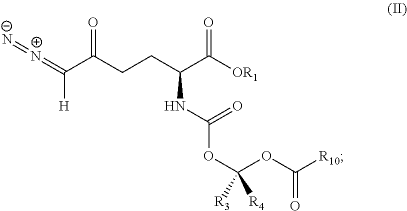
wherein:
R₁ is selected from the group consisting of H and C₁₋₆ alkyl;
R₃ and R₄ are independently selected from the group consisting of H, C₁-C₆ alkyl, substituted C₁-C₆ alkyl, aryl, and substituted aryl; and
R₁₀ is C₁₋₆ alkyl.

8. The method of claim 7, wherein R₁ is selected from the group consisting of methyl, ethyl, and isopropyl.

9. The method of claim 7, wherein R₃ is H and R₄ is selected from the group consisting of methyl and phenyl.

10. The method of claim 7, wherein R₁₀ is selected from the group consisting of isopropyl and tert-butyl.

11. The method of claim 1, wherein the prodrug is a prodrug of 6-diazo-5-oxo-norleucine (DON) and selected from the group consisting of:

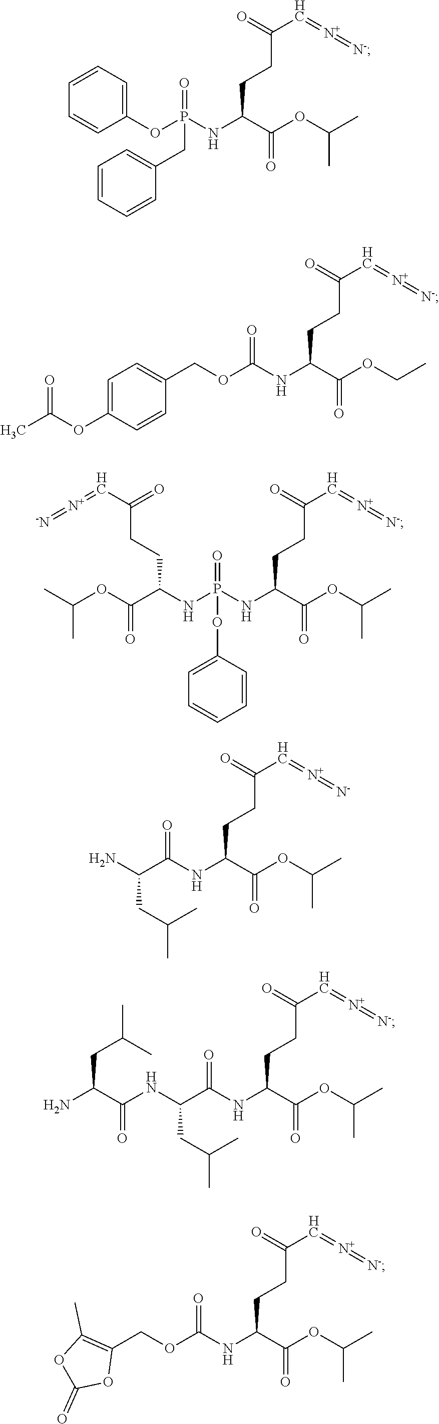

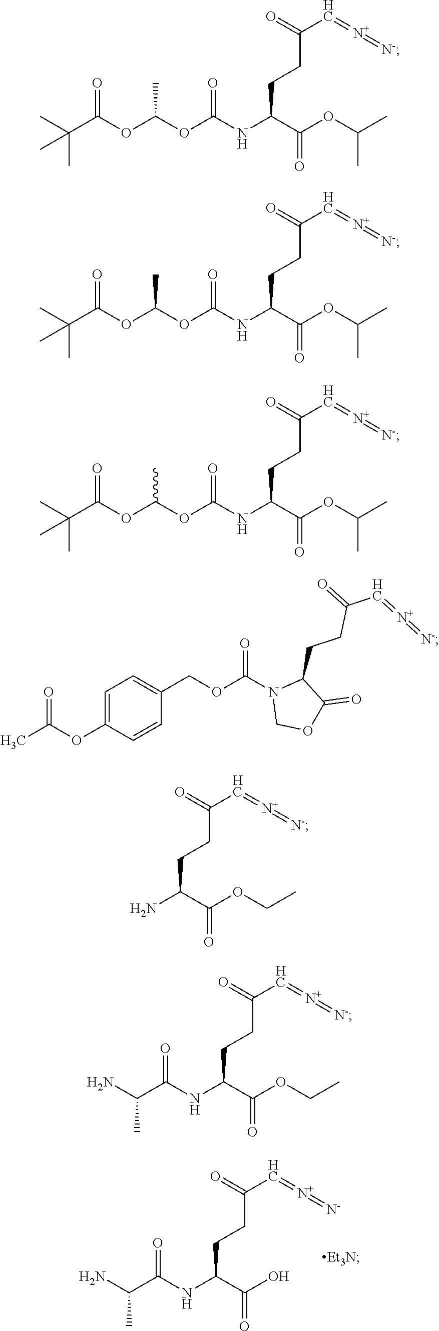

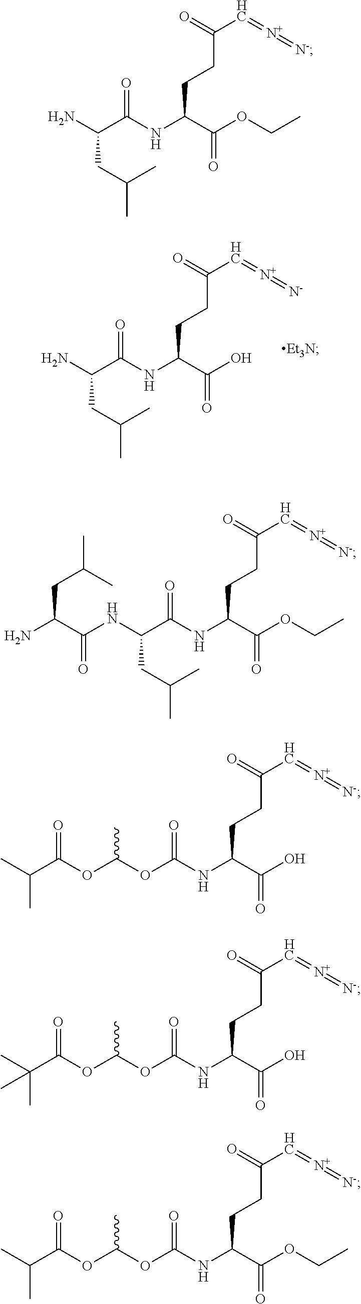

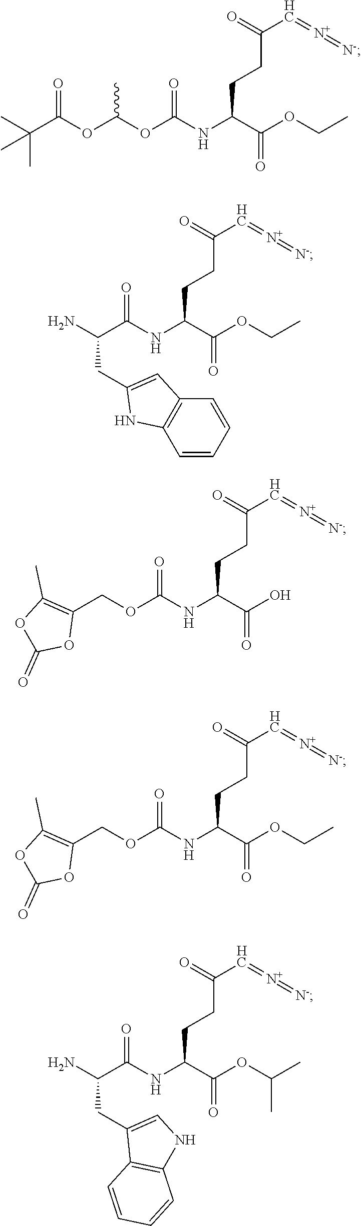

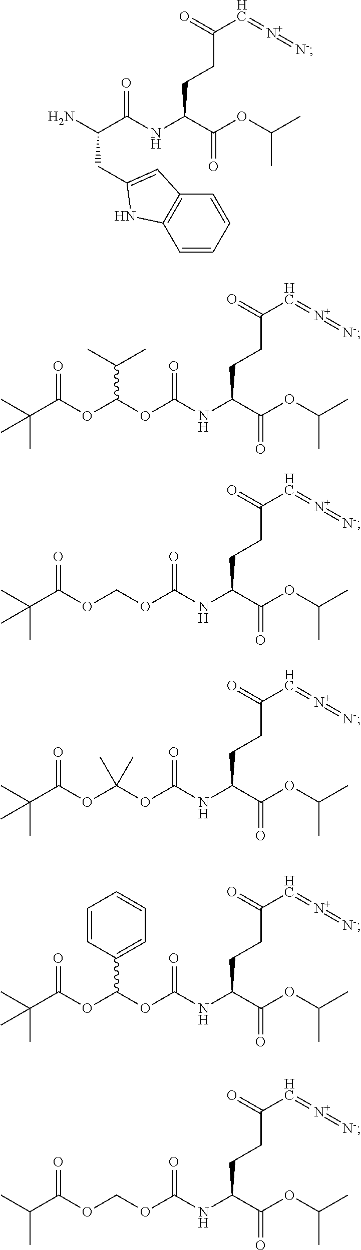

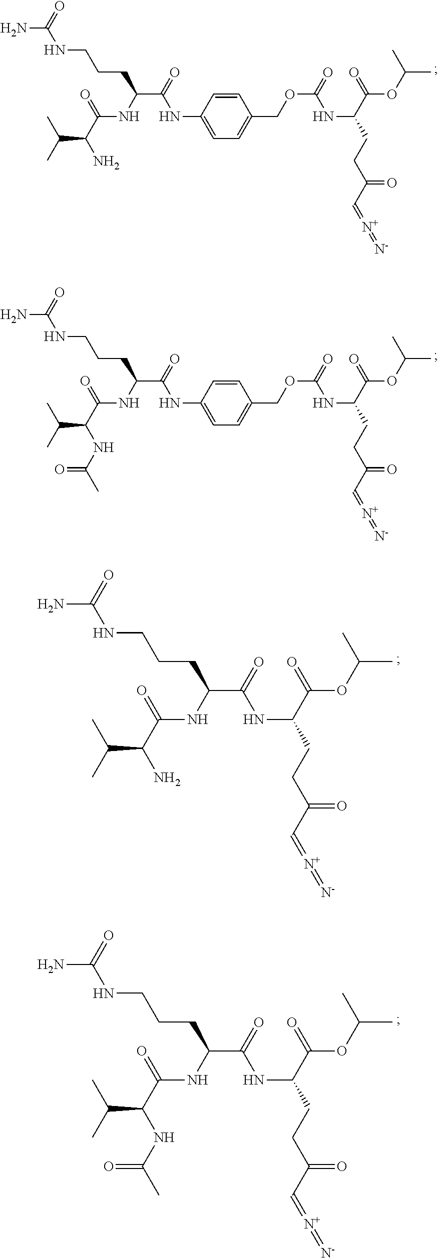 [SMILES: CC(C)OC(=O)[C@H](CCC(=O)C=[N+]=[N-])NC(=O)OCc1ccc(NC(=O)[C@H](CCCNC(N)=O)NC(=O)[C@@H](N)C(C)C)cc1 ; CC(=O)N[C@H](C(=O)N[C@@H](CCCNC(N)=O)C(=O)Nc1ccc(COC(=O)N[C@@H](CCC(=O)C=[N+]=[N-])C(=O)OC(C)C)cc1)C(C)C ; CC(C)OC(=O)[C@H](CCC(=O)C=[N+]=[N-])NC(=O)[C@H](CCCNC(N)=O)NC(=O)[C@@H](N)C(C)C ; CC(=O)N[C@H](C(=O)N[C@@H](CCCNC(N)=O)C(=O)N[C@@H](CCC(=O)C=[N+]=[N-])C(=O)OC(C)C)C(C)C]

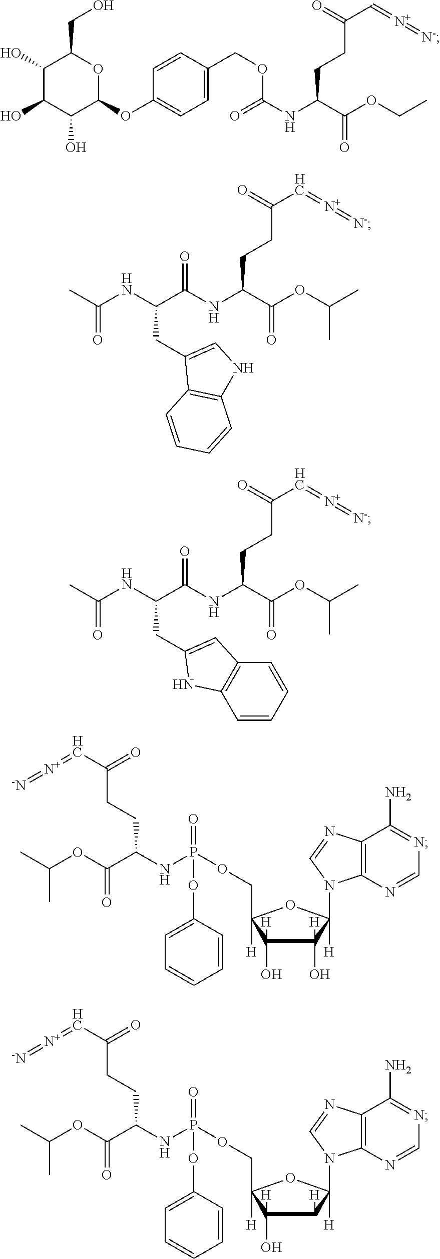

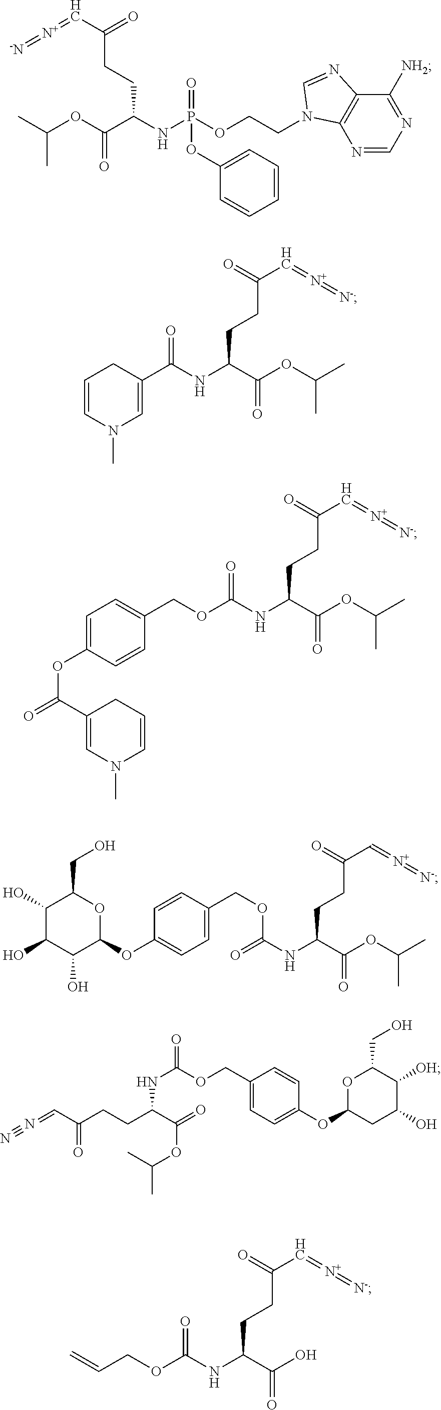

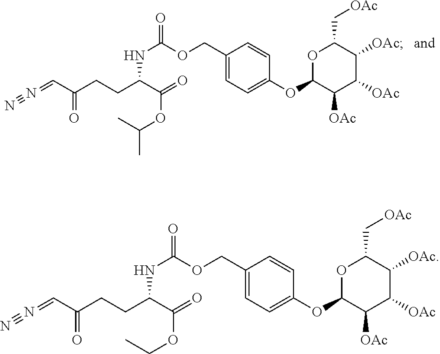

12. The method of claim 11, wherein the compound of formula (I) is:

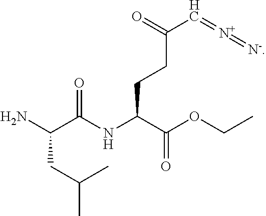

13. The method of claim 1, wherein the administering of the at least one glutamine antagonist, or a prodrug or analog thereof, modulates a glutamine pathway associated with acute kidney injury.

14. The method of claim 13, wherein the modulation of the glutamine pathway alters T cell metabolism.

15. The method of claim 13, wherein the modulation of the glutamine pathway blocks glutaminolysis.

16. The method of claim 15, wherein blockade of glutaminolysis reduces T cell activation and proliferation in post-ischemia reperfusion injury (IRI) kidneys.

17. The method of claim 15, wherein blockade of glutaminolysis reduces CD69 expression in post-ischemia reperfusion injury (IRI) kidney CD8⁺ T cells.

18. The method of claim 13, wherein the administering of the at least one glutamine antagonist, or a prodrug or analog thereof, causes a decrease in CD44, CD4⁺, and CD8⁺ T cells in post-IRI kidneys.

19. The method of claim 13, wherein the administering of the at least one glutamine antagonist, or a prodrug or analog thereof, causes a reduced expression of Ki67 cells in post-IRI kidneys.

20. The method of claim 13, the administering of the at least one glutamine antagonist, or a prodrug or analog thereof, enhances expression of hexokinase II and pS6 on one or more kidney T cells.

21. The method of claim 1, wherein the acute kidney injury is caused by ischemia or an ischemic event, direct injury to the kidney, blockage of a urinary tract, or combinations thereof.

22. The method of claim 21, wherein the direct injury to the kidney is caused by exposure to one or nephrotoxins or from a disease or condition.

23. The method of claim 22, wherein the one or more nephrotoxins include a chemotherapeutic agent, an antibiotic, an NSAID, a gold preparation, a thiazide, a sulfonamide, an aminoglycosides, an ACE inhibitor, an angiotensin II antagonist, an antiviral, vancomycin, ranitidine, amphotericin B, and combinations thereof.

24. The method of claim 22, wherein the disease or condition includes sepsis, a cancer, vasculitis, interstitial nephritis, scleroderma, tubular necrosis, glomerulonephritis, thrombotic microangiopathy, and combinations thereof.
